(19) United States
(12) Patent Application Publication
(10) Pub. No.: US 2011/0212845 A1
(43) Pub. Date: Sep. 1, 2011

(54) Biomarkers for predicting the sensitivity and response of protein kinase CK2-mediated diseases to CK2 Inhibitors

(76) Inventors: Denis DRYGIN, San Diego, CA (US); Sean E. O'BRIEN, Carlsbad, CA (US); Kenna L. ANDERES, San Diego, CA (US); Daniel VON HOFF, Scottsdale, AZ (US); John K.C. LIM, San Diego, CA (US); Claire S. PADGETT, San Diego, CA (US); Joshua R. BLIESATH, Escondido, CA (US); Caroline B. HO, Valley Center, CA (US); William G. Rice, San Diego, CA (US)

(21) Appl. No.: 12/897,640

(22) Filed: Oct. 4, 2010

Related U.S. Application Data
(60) Provisional application No. 61/248,270, filed on Oct. 2, 2009.
(60) Provisional application No. 61/255,805, filed on Oct. 28, 2009.
(60) Provisional application No. 61/323,771, filed on Apr. 13, 2010.
(60) Provisional application No. 61/380,685, filed on Sep. 7, 2010.

(51) Int. Cl. C40B 30/00; C12Q 1/48; C12Q 1/02; C12Q 1/68; G01N 33/53

(52) U.S. Cl. 506/7; 435/15; 435/29; 435/6.13; 435/7.92

(57) ABSTRACT

Disclosed are biomarkers for determining the sensitivity of protein kinase CK2-mediated diseases, such as proliferative and/or inflammatory disorders, to treatment with CK2 inhibitors. These biomarkers can be used to predict or select subjects likely to be responsive to treatment with a CK2 inhibitor, and to treat or monitor subjects undergoing treatment with a CK2 inhibitor.

### CROSS-REFERENCE TO RELATED APPLICATIONS

This application claims priority to U.S. Provisional Application Ser. No. 61/248,270, filed Oct. 2, 2009, U.S. Provisional Application Ser. No. 61/255,805, filed Oct. 28, 2009, U.S. Provisional Application Ser. No. 61/323,771, filed Apr. 13, 2010, and U.S. Provisional Application Ser. No. 61/380,685, filed Sep. 7, 2010, each of which is herein incorporated by reference in its entirety for all purposes.

### TECHNICAL FIELD

The present invention relates to biomarkers for determining the sensitivity of protein kinase CK2-mediated diseases, such as proliferative and/or inflammatory disorders, to treatment with CK2 inhibitors. Such biomarkers can be used to predict or select subjects likely to be responsive to treatment with CK2 inhibitors, and to treat or monitor subjects undergoing treatment with CK2 inhibitors.

### BACKGROUND OF THE INVENTION

Protein kinase CK2 (formerly called Casein kinase II, referred to herein as “CK2”) is a ubiquitous and highly conserved protein serine/threonine kinase. The holoenzyme is typically found in tetrameric complexes consisting of two catalytic (alpha and/or alpha′) subunits and two regulatory (beta) subunits. CK2 has a number of physiological targets and participates in a complex series of cellular functions including the maintenance of cell viability. The level of CK2 in normal cells is tightly regulated, and it has long been considered to play a role in cell growth and proliferation. Inhibitors of CK2 that are useful for treating certain types of cancers are described in PCT/US2007/077464, PCT/US2008/074820, and PCT/US2009/035609, the contents of each of which are incorporated herein by reference.

Both the prevalence and the importance of CK2 suggest it is an ancient enzyme on the evolutionary scale, as does an evolutionary analysis of its sequence; its longevity may explain why it has become important in so many biochemical processes, and why CK2 from hosts have even been co-opted by infectious pathogens (e.g., viruses, protozoa) as an integral part of their survival and life cycle biochemical systems. These same characteristics explain why inhibitors of CK2 are believed to be useful in a variety of medical treatments as discussed herein. Because it is central to many biological processes, as summarized by Guerra & Issinger, Curr. Med. Chem., 2008, 15:1870-1886, inhibitors of CK2, including the compounds described herein, should be useful in the treatment of a variety of diseases and disorders.

Cancerous cells show an elevation of CK2, and recent evidence suggests that CK2 exerts potent suppression of apoptosis in cancer cells by protecting regulatory proteins from caspase-mediated degradation. The anti-apoptotic function of CK2 may contribute to its ability to participate in transformation and tumorigenesis. In particular, CK2 has been shown to be associated with acute and chronic myelogenous leukemia, acute lymphoblastic, chronic lymphocytic leukemia, lymphoma and multiple myeloma. In addition, enhanced CK2 activity has been observed in solid tumors of the colon, rectum and breast, squamous cell carcinomas of the lung and of the head and neck (SCCHN), and adenocarcinomas of the lung, colon, rectum, kidney, breast, and prostate. Inhibition of CK2 by a small molecule is reported to induce apoptosis of pancreatic cancer cells, hepatocellular carcinoma cells (HegG2, Hep3) and cervical cancer cells (HeLa); and CK2 inhibitors dramatically sensitized RMS (Rhabdomyosarcoma) tumors toward apoptosis induced by TRAIL. Thus an inhibitor of CK2 alone, or in combination with TRAIL or a ligand for the TRAIL receptor, may be useful to treat RMS, the most common soft-tissue sarcoma in children. In addition, elevated CK2 has been found to be highly correlated with aggressiveness of neoplasias, and treatment with potent CK2 inhibitors should thus reduce the tendency of benign lesions to advance into malignant ones, or for malignant ones to metastasize.

CK2 has been found to promote signaling pathways (e.g., PI3K/Akt, NF-kB and Wnt) and cell cycle progression via phosphorylation of p21 and p27. CK2 is also reported to impair tumor suppressors (e.g., PML, PTEN, p53) and promote rRNA and tRNA biogenesis to drive protein synthesis. CK2 activates Hsp90 chaperone machinery, which may function to protect onco-kinases. These actions of CK2 may promote cancer cell survival.

Unlike other kinases and signaling pathways, where mutations are often associated with structural changes that cause loss of regulatory control, increased CK2 activity level appears to be generally caused by upregulation or overexpression of the active protein rather than by changes that affect activation levels. Guerra and Issinger postulate this may be due to regulation by aggregation, since activity levels do not correlate well with mRNA levels. Excessive activity of CK2 has been shown in many cancers, including SCCHN tumors, lung tumors, breast tumors, and others. Id.

Elevated CK2 activity in colorectal carcinomas was shown to correlate with increased malignancy. Aberrant expression and activity of CK2 have been reported to promote increased nuclear levels of NF-κB in breast cancer and myeloma cells. CK2 activity is markedly increased in patients with AML and CML during blast crisis, indicating that an inhibitor of CK2 should be particularly effective in these conditions. Multiple myeloma (MM) cell survival has been shown to rely on high activity of CK2, and inhibitors of CK2 were cytotoxic to MM cells. Similarly, a CK2 inhibitor inhibited growth of murine p190 lymphoma cells. Its interaction with Bcr/Abl has been reported to play an important role in proliferation of Bcr/Abl expressing cells, indicating inhibitors of CK2 may be useful in treatment of Bcr/Abl-positive leukemias Inhibitors of CK2 have been shown to inhibit progression of skin papillomas, prostate and breast cancer xenografts in mice, and to prolong survival of transgenic mice that express oncogenes that promote prostate cancer. Id.

The role of CK2 in various non-cancer disease processes has been recently reviewed. See Guerra & Issinger, Curr. Med. Chem., 2008, 15:1870-1886. Increasing evidence indicates that CK2 is involved in critical diseases of the central nervous system, including, for example, Alzheimer's disease, Parkinson's disease, and rare neurodegenerative disorders such as Guam-Parkinson dementia, chromosome 18 deletion syndrome, progressive supranuclear palsy, Kuf's disease, or Pick's disease. It is suggested that selective CK2-mediated phosphorylation of tau proteins may be involved in progressive neurodegeneration of Alzheimer's. In addition, recent studies suggest that CK2 plays a role in memory impairment and brain ischemia, the latter effect apparently being mediated by CK2's regulatory effect on the PI3K survival pathways.

CK2 has also been shown to be involved in the modulation of inflammatory disorders, for example, acute or chronic inflammatory pain, glomerulonephritis, and autoimmune diseases, including, e.g., multiple sclerosis (MS), systemic lupus erythematosus, rheumatoid arthritis, and juvenile arthritis. It positively regulates the function of the serotonin 5-HT3 receptor channel, activates heme oxygenase type 2, and enhances the activity of neuronal nitric oxide synthase. A selective CK2 inhibitor was reported to strongly reduce pain response of mice when administered to spinal cord tissue prior to pain testing. It phosphorylates secretory type IIA phospholipase A2 from synovial fluid of RA patients, and modulates secretion of DEK (a nuclear DNA-binding protein), which is a proinflammatory molecule found in synovial fluid of patients with juvenile arthritis. Thus inhibition of CK2 is expected to control progression of inflammatory pathologies such as those described here, and the inhibitors disclosed herein have been shown to effectively treat pain in animal models.

Protein kinase CK2 has also been shown to play a role in disorders of the vascular system, such as, e.g., atherosclerosis, laminar shear stress, and hypoxia. CK2 has also been shown to play a role in disorders of skeletal muscle and bone tissue, such as cardiomyocyte hypertrophy, impaired insulin signaling and bone tissue mineralization. In one study, inhibitors of CK2 were effective at slowing angiogenesis induced by growth factor in cultured cells. CK2 promote angiogenesis, and has been reported to activate HIF-1α under hypoxia and sustain neo-vascularization.

Moreover, in a retinopathy model, a CK2 inhibitor combined with octreotide (a somatostatin analog) reduced neovascular tufts; thus the CK2 inhibitors described herein may be effective in combination with a somatostatin analog to treat retinopathy.

CK2 has also been shown to phosphorylate GSK, troponin and myosin light chain; thus it is important in skeletal muscle and bone tissue physiology, and is linked to diseases affecting muscle tissue.

Evidence suggests that CK2 is also involved in the development and life cycle regulation of protozoal parasites, such as, for example, Theileria parva, Trypanosoma cruzi, Leishmania donovani, Herpetomonas muscarum muscarum, Plasmodium falciparum, Trypanosoma brucei, Toxoplasma gondii and Schistosoma mansoni. Numerous studies have confirmed the role of CK2 in regulation of cellular motility of protozoan parasites, essential to invasion of host cells. Activation of CK2 or excessive activity of CK2 has been shown to occur in hosts infected with Leishmania donovani, Herpetomonas muscarum muscarum, Plasmodium falciparum, Trypanosoma brucei, Toxoplasma gondii and Schistosoma mansoni. Indeed, inhibition of CK2 has been shown to block infection by T. cruzi.

CK2 has also been shown to interact with and/or phosphorylate viral proteins associated with human immunodeficiency virus type 1 (HIV-1), human papilloma virus, and herpes simplex virus, in addition to other virus types (e.g. human cytomegalovirus, hepatitis C and B viruses, Borna disease virus, adenovirus, coxsackievirus, coronavirus, influenza, and varicella zoster virus). CK2 phosphorylates and activates HIV-1 reverse transcriptase and proteases in vitro and in vivo, and promotes pathogenicity of simian-human immunodeficiency virus (SHIV), a model for HIV. Inhibitors of CK2 are thus able to reduce pathogenic effects of a model of HIV infection. CK2 also phosphorylates numerous proteins in herpes simplex virus and numerous other viruses, and some evidence suggests viruses have adopted CK2 as a phosphorylating enzyme for their essential life cycle proteins. Inhibition of CK2 is thus expected to deter infection and progression of viral infections, which rely upon the host's CK2 for their own life cycles.

CK2 is unusual in the diversity of biological processes that it affects, and it differs from most kinases in other ways as well: it is constitutively active, it can use ATP or GTP, and it is elevated in most tumors and rapidly proliferating tissues. It also has unusual structural features that may distinguish it from most kinases, too, enabling its inhibitors to be highly specific for CK2 while many kinase inhibitors affect multiple kinases, increasing the likelihood of off-target effects, or variability between individual subjects. For all of these reasons, CK2 is a particularly interesting target for drug development, and the invention provides highly effective inhibitors of CK2 that are useful in treating a variety of different diseases and disorders mediated by or associated with excessive, aberrant or undesired levels of CK2 activity.

It has been postulated that overexpression of CK2 is a negative prognostic marker for cancer (Ahmad et al, 2005; Duncan & Litchfeld 2008). In addition, although the phosphorylation of Akt at Serine 129 by CK2 has been described in the literature (Di Maira et al., 2005; Di Maira et al., 2009), the way in which a potential CK2 inhibitor would affect Akt phosphorylation is unknown and not yet predictable.

IL-6 and IL-8 are well-described inflammatory response mediators. IL-6 is pro-inflammatory cytokine known to play a role in inflammatory diseases and cancer. IL-6 serves as autocrine and paracrine growth factors for several cancers, and high levels of IL-6 correlate with a poor prognosis and increased production of angiogenic factors. IL-8 is a chemokine produced by macrophages, epithelial cells and other cell types, and is a major mediator of the inflammatory response. IL-8 functions as a chemoattractant and is also a potent angiogenic factor.

CK2 has been reported to phosphorylate and, thereby, modulate the activity of transcription factors involved in regulation of the inflammatory response, including, e.g., nuclear factor-kappa B (NF-κB), signal transducer and activator of transcription (STAT)1, cyclic adenosine monophosphate (cAMP) response element binding protein (CREB), cAMP response element modulator protein (CREM), PU.1, specificity protein-1 (Sp1), CCAAT-enhancer binding proteins (C/EBP), steroid hormone receptors, and the protooncogenes c-Jun, c-Fos, c-Myc, and Max. See Singh & Ramji, J. Mol. Med. 2008, 86(8):887-97.

Inflammatory breast cancer (IBC) exhibits increased angiogenesis and lymphangiogenesis and has a higher metastatic potential than noninflammatory breast cancer. While the role of CK2 in breast cancer in general has been investigated, there is no literature describing the role of CK2 in IBC.

CK2 regulates NF-κB transcription via phosphorylation of IκB and NF-κB. IL-6 and IL-8 are NF-κB target genes. While CK2 is known to be involved in regulation of NF-κB, one of the transcriptional factors responsible for expression of IL-6, the link between CK2 and IL-6 is not well established. The potential regulation of IL-8 through NF-kB in intestine has been reported (Parhar et al., 2007).

Cluster of differentiation 19 (CD19) is expressed on follicular dendritic cells and B cells. CD19 is present on B cells from earliest recognizable B-lineage cells during development to B-cell blasts, but is lost upon maturation to plasma cells. After activation, the cytoplasmic tail of CD19 becomes phosphorylated which leads to binding by Src-family kinases and recruitment of PI-3 kinase. Mutations causing defects in the development of B cells can give rise to cancers such as lymphomas and leukemias. CD19 has been shown to be a major regulator of AKT activity (Otero, Omori & Rickert, 2001) and constitutive activation of Akt contributes to the pathogenesis and survival of multiple B-cell-derived diseases including mantle cell lymphoma (Radelius, Pittaluga, Nishizuka et al., 2006).

As described above, CK2 inhibitors have been found to possess potent antiproliferative properties which make them useful for cancer chemotherapy. However, there is a need for more targeted use of CK2 inhibitors which requires identification of subjects who are likely to respond to treatment with such agents. The identification of biomarkers useful to predict the responsiveness of a cell, tissue, tumor or subject to treatment with CK2 inhibitors is extremely valuable in developing targeted approaches for the treatment of CK2-mediated disorders, including, but not limited to, proliferative disorders such as cancers. Such biomarkers may be used as criteria to identify and/or select patients likely to receive a therapeutic benefit from administration of a CK2 inhibitor. Moreover, these and other biomarkers can also useful for monitoring the response of a subject to treatment, and to determine whether to modify the dosing regimen, or to replace or augment the therapeutic agent.

Accordingly, there is a need to identify biomarkers which are capable of predicting the sensitivity and/or monitoring the response of a CK2-mediated disease, such as a proliferative disorder and/or an inflammatory disorder, to treatment with a CK2 inhibitor.

### SUMMARY OF THE INVENTION

The present invention relates to biomarkers for predicting, determining and/or monitoring the sensitivity of a CK2-mediated disease, such as a proliferative disorder and/or an inflammatory disorder, to treatment with a therapeutic agent, in particular a CK2 inhibitor.

In a first aspect, the present invention provides biomarkers that are useful for predicting the sensitivity and/or responsiveness of a subject or system to treatment with a CK2 inhibitor. The biomarkers and associated methods of measuring said biomarkers can be used to select an individual subject or a population of subjects for treatment with a particular CK2 inhibitor. The invention also relates to the use of these biomarkers to monitor or predict the outcome of treatment in subjects being administered a CK2 inhibitor.

As described herein, biomarkers useful for predicting the sensitivity and/or monitoring the responsiveness of a CK2-mediated disease to treatment with a CK2 inhibitor include the mRNA expression and/or polypeptide levels (i.e., the protein expression) of IL-6, IL-8, HIF-1α, VEGF, CK2α and/or CK2α′ subunits, CK2β, and the level of phosphorylated Akt serine 129 (p-Akt S129), alone or relative to total Akt polypeptide (i.e., the normalized level of p-Akt S129). Additional biomarkers include the level of phosphorylated Akt serine 473 (p-Akt S473), alone or relative to total Akt polypeptide (i.e., the normalized level of p-Akt S473), the level of phosphorylated p21 threonine 145 (p-p21 T145), alone or relative to total p21 polypeptide (i.e., the normalized level of p-p21 T145), the level of phosphorylated nuclear factor-κB (NF-κB) serine 529 (p-NF-κB S529), alone or relative to total NF-κB polypeptide (i.e., the normalized level of p-NF-κB S529), the level of phosphorylated STAT3 tyrosine 705 (p-STAT3 Y705), alone or relative to total STAT3 polypeptide (i.e., the normalized level of p-STAT3 Y705), or the level of phosphorylated JAK2 tyrosine 1007/1008 (p-JAK2 Y1007/1008), alone or relative to total JAK2 polypeptide (i.e., the normalized level of p-JAK2 Y1007/1008).

Accordingly, in a second aspect, the invention provides methods for predicting the sensitivity and/or monitoring the responsiveness of a CK2-mediated disease, such as a proliferative disorder and/or an inflammatory disorder, in a subject to treatment with a CK2 inhibitor, comprising determining the mRNA expression and/or polypeptide levels of one or more biomarkers selected from IL-6, IL-8, HIF-1α, VEGF, CK2α and CK2α′, CK2β, and/or the level of phosphorylation for p-Akt S129, p-Akt S473, p-p21 T145, p-NF-κB S529, p-STAT3 Y705, p-JAK2 Y1007/1008, alone or relative to the total level of unphosphorylated protein (i.e. the normalized level) in a biological sample derived from the subject, as further described herein.

In one such embodiment, the method comprises determining the level of IL-6 mRNA expression and/or IL-6 polypeptide in a biological sample derived from the subject, wherein an increase in the level of IL-6 mRNA expression and/or IL-6 polypeptide relative to control is predictive of the sensitivity of the CK2-mediated disease to treatment with a CK2 inhibitor.

In another such embodiment, the method comprises determining the level of IL-6 mRNA expression and/or IL-6 polypeptide in a first biological sample derived from the subject prior to administration with a CK2 inhibitor, wherein a decrease in the level of IL-6 mRNA expression and/or IL-6 polypeptide relative to a second biological sample derived from the subject following administration of the CK2 inhibitor is indicative of a positive response to treatment of the CK2-mediated disease to treatment with a CK2 inhibitor.

In another such embodiment, the method comprises determining the level of IL-8 mRNA expression and/or IL-8 polypeptide in a biological sample derived from the subject, wherein an increase in the level of IL-8 mRNA expression and/or IL-8 polypeptide relative to control is predictive of the sensitivity of the CK2-mediated disease to treatment with a CK2 inhibitor.

In another such embodiment, the method comprises determining the level of IL-8 mRNA expression and/or IL-8 polypeptide in a first biological sample derived from the subject prior to administration with a CK2 inhibitor, wherein a decrease in the level of IL-8 mRNA expression and/or IL-8 polypeptide relative to a second biological sample derived from the subject following administration of the CK2 inhibitor is indicative of a positive response to treatment of the CK2-mediated disease to treatment with a CK2 inhibitor.

In another such embodiment, the method comprises determining the level of CK2α mRNA expression and/or CK2α polypeptide in a biological sample derived from the subject, wherein an increase in the level of CK2α mRNA expression and/or CK2α polypeptide relative to control is predictive of the sensitivity of the CK2-mediated disease to treatment with a CK2 inhibitor.

In another such embodiment, the method comprises determining the level of CK2α mRNA expression and/or CK2α polypeptide in a first biological sample derived from the subject prior to administration with a CK2 inhibitor, wherein a decrease in the level of CK2α mRNA expression and/or CK2α polypeptide relative to a second biological sample derived from the subject following administration of the CK2 inhibitor is indicative of a positive response to treatment of the CK2-mediated disease to treatment with a CK2 inhibitor.

In another such embodiment, the method comprises determining the level of CK2α′ mRNA expression and/or CK2α′ polypeptide in a biological sample derived from the subject, wherein an increase in the level of CK2α′ mRNA expression and/or CK2α′ polypeptide relative to control is predictive of the sensitivity of the CK2-mediated disease to treatment with a CK2 inhibitor.

In another such embodiment, the method comprises determining the level of CK2α′ mRNA expression and/or CK2α′ polypeptide in a first biological sample derived from the subject prior to administration with a CK2 inhibitor, wherein a decrease in the level of CK2α′ mRNA expression and/or CK2α′ polypeptide relative to a second biological sample derived from the subject following administration of the CK2 inhibitor is indicative of a positive response to treatment of the CK2-mediated disease to treatment with a CK2 inhibitor.

In another such embodiment, the method comprises determining the level of VEGF mRNA expression and/or VEGF polypeptide in a biological sample derived from the subject, wherein an increase in the level of VEGF mRNA expression and/or VEGF polypeptide relative to control is predictive of the sensitivity of the CK2-mediated disease to treatment with a CK2 inhibitor.

In another such embodiment, the method comprises determining the level of VEGF mRNA expression and/or VEGF polypeptide in a first biological sample derived from the subject prior to administration with a CK2 inhibitor, wherein a decrease in the level of VEGF mRNA expression and/or VEGF polypeptide relative to a second biological sample derived from the subject following administration of the CK2 inhibitor is indicative of a positive response to treatment of the CK2-mediated disease to treatment with a CK2 inhibitor.

In another such embodiment, the method comprises determining the level of CK2α mRNA expression and/or CK2α polypeptide in a biological sample derived from the subject; and determining the level of phosphorylated Akt S129 (p-Akt S129) polypeptide in a biological sample derived from the subject, wherein a positive correlation between the level of CK2α mRNA expression and/or CK2α polypeptide and the level of p-Akt S129 polypeptide is predictive of sensitivity of the CK2-mediated disease to treatment with a CK2 inhibitor.

In a further embodiment, the method comprises determining the level of CK2α′ mRNA expression and/or CK2α′ polypeptide in a biological sample derived from the subject; and determining the level of phosphorylated Akt S129 (p-Akt S129) polypeptide relative to the level of total Akt polypeptide in a biological sample derived from the subject, wherein a positive correlation between the level of CK2α′ mRNA expression and/or CK2α′ polypeptide and the normalized level of p-Akt S129 polypeptide is predictive of sensitivity of the CK2-mediated disease to treatment with a CK2 inhibitor.

In another embodiment, the method comprises determining the level of phosphorylated Akt S129 (p-Akt S129) polypeptide in a biological sample derived from the subject, wherein an increase in the level of p-Akt S129 polypeptide relative to control is predictive of the sensitivity of the CK2-mediated disease to treatment with a CK2 inhibitor.

In another embodiment, the method comprises determining the level of phosphorylated Akt S129 (p-Akt S129) polypeptide in a first biological sample derived from the subject prior to administration with a CK2 inhibitor, wherein a decrease in the level of phosphorylated Akt S129 (p-Akt S129) polypeptide relative to a second biological sample derived from the subject following administration of the CK2 inhibitor is indicative of a positive response to treatment of the CK2-mediated disease to treatment with a CK2 inhibitor.

In yet another embodiment, the method comprises determining the level of phosphorylated Akt S129 (p-Akt S129) polypeptide relative to the level of total Akt polypeptide in a biological sample derived from the subject, wherein an increase in the normalized level of p-Akt S129 polypeptide relative to the corresponding control is predictive of the sensitivity of the CK2-mediated disease to treatment with a CK2 inhibitor.

In yet another embodiment, the method comprises determining the level of phosphorylated Akt S129 (p-Akt S129) polypeptide relative to the level of total Akt polypeptide in a first biological sample derived from the subject prior to administration with a CK2 inhibitor, wherein a decrease in the level of phosphorylated Akt S129 (p-Akt S129) polypeptide relative to the level of total Akt polypeptide as compared to a second biological sample derived from the subject following administration of the CK2 inhibitor is indicative of a positive response to treatment of the CK2-mediated disease to treatment with a CK2 inhibitor.

In another embodiment, the method comprises determining the level of phosphorylated Akt S473 (p-Akt S473) polypeptide in a biological sample derived from the subject, wherein an increase in the level of p-Akt S473 polypeptide relative to control is predictive of the sensitivity of the CK2-mediated disease to treatment with a CK2 inhibitor.

In another embodiment, the method comprises determining the level of phosphorylated Akt S473 (p-Akt S473) polypeptide in a first biological sample derived from the subject prior to administration with a CK2 inhibitor, wherein a decrease in the level of phosphorylated Akt S473 (p-Akt S473) polypeptide relative to a second biological sample derived from the subject following administration of the CK2 inhibitor is indicative of a positive response to treatment of the CK2-mediated disease to treatment with a CK2 inhibitor.

In yet another embodiment, the method comprises determining the level of phosphorylated Akt S473 (p-Akt S473) polypeptide relative to the level of total Akt polypeptide in a biological sample derived from the subject, wherein an increase in the normalized level of p-Akt S473 polypeptide relative to the corresponding control is predictive of the sensitivity of the CK2-mediated disease to treatment with a CK2 inhibitor.

In yet another embodiment, the method comprises determining the level of phosphorylated Akt S473 (p-Akt S473) polypeptide relative to the level of total Akt polypeptide in a first biological sample derived from the subject prior to administration with a CK2 inhibitor, wherein a decrease in the level of phosphorylated Akt S473 (p-Akt S473) polypeptide relative to the level of total Akt polypeptide as compared to a second biological sample derived from the subject following administration of the CK2 inhibitor is indicative of a positive response to treatment of the CK2-mediated disease to treatment with a CK2 inhibitor.

In another embodiment, the method comprises determining the level of phosphorylated p21 T 145(phospho-p21 T145 or p-p21 T145) polypeptide in a biological sample derived from the subject, wherein an increase in the level of p-p21T145 polypeptide relative to control is predictive of the sensitivity of the CK2-mediated disease to treatment with a CK2 inhibitor.

In another embodiment, the method comprises determining the level of phosphorylated p21 T 145(phospho-p21 T145 or p-p21 T145) polypeptide in a first biological sample derived from the subject prior to administration with a CK2 inhibitor, wherein a decrease in the level of phosphorylated p21 T 145(phospho-p21 T145 or p-p21 T145) polypeptide relative to a second biological sample derived from the subject following administration of the CK2 inhibitor is indicative of a positive response to treatment of the CK2-mediated disease to treatment with a CK2 inhibitor.

In yet another embodiment, the method comprises determining the level of phosphorylated p21 T 145(phospho-p21 T145 or p-p21 T145) polypeptide relative to the level of total p21 polypeptide in a biological sample derived from the subject, wherein an increase in the normalized level of p-p21 polypeptide relative to the corresponding control is predictive of the sensitivity of the CK2-mediated disease to treatment with a CK2 inhibitor.

In yet another embodiment, the method comprises determining the level of phosphorylated p21 T 145(phospho-p21 T145 or p-p21 T145) polypeptide relative to the level of total p21 polypeptide in a first biological sample derived from the subject prior to administration with a CK2 inhibitor, wherein a decrease in the level of phosphorylated p21 T 145(phospho-p21 T145 or p-p21 T145) polypeptide relative to the level of total p21 polypeptide as compared to a second biological sample derived from the subject following administration of the CK2 inhibitor is indicative of a positive response to treatment of the CK2-mediated disease to treatment with a CK2 inhibitor.

In another embodiment, the method comprises determining the level of phosphorylated nuclear factor-κB (NF-κB) serine 529 (p-NF-κB S529) polypeptide in a biological sample derived from the subject, wherein an increase in the level of p-NF-κB S529 polypeptide relative to control is predictive of the sensitivity of the CK2-mediated disease to treatment with a CK2 inhibitor.

In another embodiment, the method comprises determining the level of phosphorylated nuclear factor-κB (NF-κB) serine 529 (p-NF-κB S529) polypeptide in a first biological sample derived from the subject prior to administration with a CK2 inhibitor, wherein a decrease in the level of phosphorylated NF-κB S529 (p-NF-κB S529) polypeptide relative to a second biological sample derived from the subject following administration of the CK2 inhibitor is indicative of a positive response to treatment of the CK2-mediated disease to treatment with a CK2 inhibitor.

In yet another embodiment, the method comprises determining the level of phosphorylated nuclear factor-κB (NF-κB) serine 529 (p-NF-κB S529) polypeptide relative to the level of total NF-κB polypeptide in a biological sample derived from the subject, wherein an increase in the normalized level of p-NF-κB S529 polypeptide relative to the corresponding control is predictive of the sensitivity of the CK2-mediated disease to treatment with a CK2 inhibitor.

In yet another embodiment, the method comprises determining the level of phosphorylated nuclear factor-κB (NF-κB) serine 529 (p-NF-κB S529) polypeptide relative to the level of total NF-κB polypeptide in a first biological sample derived from the subject prior to administration with a CK2 inhibitor, wherein a decrease in the level of phosphorylated NF-κB S529 (p-NF-κB S529) polypeptide relative to the level of total NF-κB polypeptide as compared to a second biological sample derived from the subject following administration of the CK2 inhibitor is indicative of a positive response to treatment of the CK2-mediated disease to treatment with a CK2 inhibitor.

In another embodiment, the method comprises determining the level of phosphorylated STAT3 tyrosine 705 (p-STAT3 Y705) polypeptide in a biological sample derived from the subject, wherein an increase in the level of p-STAT3 Y705 polypeptide relative to control is predictive of the sensitivity of the CK2-mediated disease to treatment with a CK2 inhibitor.

In another embodiment, the method comprises determining the level of phosphorylated STAT3 tyrosine 705 (p-STAT3 Y705) polypeptide in a first biological sample derived from the subject prior to administration with a CK2 inhibitor, wherein a decrease in the level of phosphorylated STAT3 tyrosine 705 (p-STAT3 Y705) polypeptide relative to a second biological sample derived from the subject following administration of the CK2 inhibitor is indicative of a positive response to treatment of the CK2-mediated disease to treatment with a CK2 inhibitor.

In yet another embodiment, the method comprises determining the level of phosphorylated STAT3 tyrosine 705 (p-STAT3 Y705) polypeptide relative to the level of total STAT3 polypeptide in a biological sample derived from the subject, wherein an increase in the normalized level of p-STAT3 Y705 polypeptide relative to the corresponding control is predictive of the sensitivity of the CK2-mediated disease to treatment with a CK2 inhibitor.

In yet another embodiment, the method comprises determining the level of phosphorylated STAT3 tyrosine 705 (p-STAT3 Y705) polypeptide relative to the level of total STAT3 polypeptide in a first biological sample derived from the subject prior to administration with a CK2 inhibitor, wherein a decrease in the level of phosphorylated STAT3 tyrosine 705 (p-STAT3 Y705) polypeptide relative to the level of total STAT3 polypeptide as compared to a second biological sample derived from the subject following administration of the CK2 inhibitor is indicative of a positive response to treatment of the CK2-mediated disease to treatment with a CK2 inhibitor.

In another embodiment, the method comprises determining the level of phosphorylated JAK2 tyrosine 1007/1008 (p-JAK2 Y1007/1008) polypeptide in a biological sample derived from the subject, wherein an increase in the level of p-JAK2 Y1007/1008 polypeptide relative to control is predictive of the sensitivity of the CK2-mediated disease to treatment with a CK2 inhibitor.

In another embodiment, the method comprises determining the level of phosphorylated JAK2 tyrosine 1007/1008 (p-JAK2 Y1007/1008) polypeptide in a first biological sample derived from the subject prior to administration with a CK2 inhibitor, wherein a decrease in the level of phosphorylated JAK2 tyrosine 1007/1008 (p-JAK2 Y1007/1008) polypeptide relative to a second biological sample derived from the subject following administration of the CK2 inhibitor is indicative of a positive response to treatment of the CK2-mediated disease to treatment with a CK2 inhibitor.

In yet another embodiment, the method comprises determining the level of phosphorylated JAK2 tyrosine 1007/1008 (p-JAK2 Y1007/1008) polypeptide relative to the level of total JAK2 polypeptide in a biological sample derived from the subject, wherein an increase in the normalized level of p-JAK2 Y1007/1008 polypeptide relative to the corresponding control is predictive of the sensitivity of the CK2-mediated disease to treatment with a CK2 inhibitor.

In yet another embodiment, the method comprises determining the level of phosphorylated JAK2 tyrosine 1007/1008 (p-JAK2 Y1007/1008) polypeptide relative to the level of total JAK2 polypeptide in a first biological sample derived from the subject prior to administration with a CK2 inhibitor, wherein a decrease in the level of phosphorylated JAK2 tyrosine 1007/1008 (p-JAK2 Y1007/1008) polypeptide relative to the level of total JAK2 polypeptide as compared to a second biological sample derived from the subject following administration of the CK2 inhibitor is indicative of a positive response to treatment of the CK2-mediated disease to treatment with a CK2 inhibitor.

In various embodiments described herein, the biological sample may be selected from a cell, a tissue, a tissue culture, a tumor, or a biological fluid derived from said subject. In one embodiment, the biological fluid may be selected from plasma, serum, or peripheral blood mononuclear cells (PBMCs).

In various embodiments described herein, the proliferative disorder is a cancer or malignancy. In one embodiment, the cancer or malignancy may be head & neck cancer, non-small cell lung carcinoma (NSCLC), breast cancer including inflammatory breast cancer (IBC), prostate cancer, pancreatic cancer, lymphomas including non-Hodgkins lymphoma (NHL) and Mantle cell lymphoma (MCL), glioblastoma, squamous cell carcinoma (SCC) of the lung, ovarian cancer, multiple myeloma, acute myeloid leukemia, colorectal cancer, and thyroid cancer.

In frequent embodiments, the CK2-mediated disease is a proliferative disorder and/or an inflammatory disorder, and the methods are used to determine the sensitivity of such disorders to treatment with a CK2 inhibitor. In specific embodiments, the CK2 inhibitor is CX-4945 or an analog thereof, including, but not limited to, Compound 1 and Compound 2.

In some embodiments, the method comprises determining the mRNA expression and/or polypeptide levels using two or more of the above-mentioned biomarkers.

The invention also relates to the use of the above described methods to select subjects suffering from a CK2-mediated disease, such as a proliferative disorder and/or an inflammatory disorder, for treatment with a CK2 inhibitor, and to methods of treating subjects selected using these methods.

Thus, in another aspect, the invention provides a method for selecting subjects suffering from a CK2-mediated disease, such as a proliferative disorder and/or an inflammatory disorder, for treatment with a CK2 inhibitor, comprising predicting the sensitivity of the CK2-mediated disorder to treatment with a CK2 inhibitor in each subject by determining the levels of one or more biomarkers, as described herein, and selecting those subjects showing the response indicated as predictive of sensitivity for treatment with a CK2 inhibitor.

In some embodiments, the methods provided herein may be used to identify or select a patient or population of patients likely to benefit from treatment with a CK2 inhibitor. In other embodiments, the methods may be useful to identify patients unlikely to benefit from treatment with a CK2 inhibitor. Such methods may also be used to select a population of patients for inclusion (or exclusion) in a clinical trial to assess the efficacy of treatment with a CK2 inhibitor. The methods described herein may also be used to assess the response of patients undergoing treatment with a CK2 inhibitor, and thus may be useful to monitor or predict the outcome of treatment with a CK2 inhibitor.

In a further aspect, the invention provides a method for treating a CK2-mediated disease, such as a proliferative disorder and/or an inflammatory disorder, in a subject in need thereof, comprising determining the levels of one or more biomarkers in a biological sample derived from the subject, as described herein, and treating the subject with a CK2 inhibitor if the level of the biomarker in the subject's sample provides the response indicated to be predictive of sensitivity or responsiveness to treatment with a CK2 inhibitor.

These and other embodiments of the invention are described herein.

### BRIEF DESCRIPTION OF THE DRAWINGS

FIG. 1 illustrates the effect of IL-6 in multiple myeloma cells. IL-6 induces VEGF (vascular endothelial growth factor) secretion, which promotes angiogenesis, stimulates growth and migration of multiple myeloma cells, further augments IL-6 secretion, and prevents antigen presentation by dendritic cells.

FIG. 2 shows the inhibitory activity of the CK2 inhibitor, CX-4945, in comparison to various CX-4945 analogs.

FIG. 3 shows the differential sensitivity of CX-4945 between cancerous cells and normal cells. The Y-axis shows the fold-induction of Caspase 3/7 activity, a marker of cell apoptosis. The X-axis illustrates the cell type. BT-474: breast cancer cells; Mia PaCa 2 and BxPC3-3: pancreatic cancer cells; SK-OV-3 and A2780: ovarian cancer cells; A375: melanoma cells; CCD18Co: normal colon fibroblast cells; CCD1058 and CCD1068: normal skin fibroblast cells; and Mrc5 and IMR90: normal lung fibroblast cells.

FIG. 4 illustrates the inhibition of tumor growth following treatment with CX-4945 (25 mg/kg bid or 75 mg/kg bid) over the course of 35 days.

FIG. 5A illustrates the inhibition of breast cancer tumor growth in the BT-474 breast cancer cell line following treatment with CX-4945 (25 mg/kg bid or 75 mg/kg bid) over the course of 35 days.

FIG. 5B illustrates the inhibition of ovarian cancer tumor growth in the SK-OV-3 ovarian cancer cell line following treatment with CX-4945 (25 mg/kg bid or 75 mg/kg bid) over the course of 35 days.

FIG. 6 illustrates the inhibition of pancreatic cancer tumor growth in BxPC3 pancreatic cancer xenografts following treatment with CX-4945 (12.5 mg/kg bid, 25 mg/kg bid, 50 mg/kg bid, or 75 mg/kg bid) over the course of 35 days. The drug was well tolerated and plasma concentrations of CX-4945 were closely correlated with the dosing regimen.

FIG. 7 shows IL-6 and IL-8 levels in plasma on day 21 relative to day 1 following treatment with CX-4945 (CX-4945).

FIG. 8 shows the percent change in IL-6 and IL-8 levels following 21 days of treatment with CX-4945 (CX-4945) in patients with NSCLC, prostate, thyroid/papillary and Leydig cell tumors.

FIG. 9A shows the percent change in serum IL-6 levels following 21 days of treatment with CX-4945 in Cohorts 1-3 of Example 1.

FIG. 9B shows the IL-6 levels in patient ID NOs: 1-24 following 1 and 21 days of treatment with CX-4945.

FIG. 10 shows the IL-8 levels in patient ID NOs: 1-24 following 1 and 21 days of treatment with CX-4945.

FIG. 11 shows the percent change in Akt S473/Akt 8 hours post-dose on day 1 and day 21 in CD19 PBMCs following treatment with CX-4945 in Cohorts 1-3 of Example 1.

FIG. 12 shows the percent change in p21 T145/p21 4 hours post-dose on day 1 and day 21 in CD45 PBMCs following treatment with CX-4945 in Cohorts 1-3 of Example 1.

FIG. 13 shows the change in p-Akt S129 (A), p-Akt S473 (B), and p-p21 T145 (C) between pre-dose (time=0) and steady state (time=8 days or 21 days) time points as a function of cumulative AUC.

FIG. 14 shows the change in p-Akt S129 in circulating tumor cells (CTCs) between pre-dose (time=0) and 6 hours post dose on day 8 time points for patients on the QID schedule.

FIG. 15 shows the secretion of IL-6 by SUM-149PT inflammatory breast cancer (IBC) cells treated for six hours with CX-4945 concentrations from 0.05 μM up to 50 μM (A). Cell viability of the SUM-149PT cells was determined after 96 hours (B).

FIG. 16 shows the effect of CX-4945 on secretion of IL-6 by aggressive SUM-149PT xenografts. Effect on tumor weight is shown in panel (A). Aggressive tumors (larger than 1 g) were found to have a higher rate of IL-6 secretion than smaller tumors (B). CX-4945 was found to reduce IL-6 secretion in all tumors (C) and to significantly reduce IL-6 secretion by aggressive tumors (D).

FIG. 17 shows the effects of treatment in mice bearing SUM-149PT xenografts, left untreated (UTC) or treated PO once (one time) or BID for 8 days (xD8) with 75 mg/kg of CX-4945.

FIG. 18 shows the expression of Akt S129 in untreated cells (UTC) and cells treated with CX-4945 and additional chemotherapeutic agents, including 5-fluorouracil (5-FU), BEZ 235, AZD 6244, erlotinib, lapatinib, sorafenib, and sunitinib (Sutent).

FIG. 19 shows the phosphorylation status of p21 at T145 and Akt at S129 following treatment with 10 μM of CX-4945 at 4 hours and 8 hours, compared to reversible washout conditions.

FIG. 20 shows the relationship between CK2α mRNA levels (RU) and compound IC₅₀ (μM) in breast cancer cells for CX-4945 (A), Compound 1 (B) and Compound 2 (C)

FIG. 21 shows the correlation between CK2α′ subunit level and Akt S129 phosphorylation status in breast cancer cell lines that are sensitive and resistant to CX-4945 and Compound 2 (A), and levels for CK2α′ and p-Akt S129 in various breast cancer cell lines (B).

FIG. 22 shows phospho-protein levels in PBMCs at 4 hours post dose on day 21 versus pre-treatment with CX-4945 for biomarkers (A) Akt S 129, (B) Akt S473 and (C) p21 T145.

FIG. 23 shows predicted versus calculated IC₅₀ values for CX-4945 using CK2α and normalized pAkt S129 markers (A) and polypeptide levels of CK2α and p-Akt S129 (B).

FIG. 24 shows the effect of increasing concentrations of CX-4945 on PIK3/Akt signaling and cell cycle progression as evaluated in BT-474 breast cancer and BxPC-3 pancreatic cancer cells.

FIG. 25 illustrates the ability of CX-4945 to modulate the cell cycle in BT-474 breast cancer cells and BxPC-3 pancreatic cancer cells.

FIG. 26 illustrates the effects of increasing concentrations of CX-4945 on tube formation and migration in BxPC-3 cells.

FIG. 27 shows the effect of CX-4945 on concentrations of aldolase, pVHL, and p53.

FIG. 28 illustrates a luciferase reporter assay used to measure the expression of HIF-1α following exposure to increasing concentrations of CX-4945.

FIG. 29 shows the expression of CK2 mRNA (A) and CK2 protein (B) in a panel of human multiple myeloma cell lines.

FIG. 30 shows an in vitro kinase assay which demonstrates the effect of CX-4945 on CK2 activity in several multiple myeloma cell lines.

FIG. 31 illustrates how CX-4945 modulates several key proteins in human multiple myeloma cells, including Akt1 (A), NF-κB (B), JAK2/STAT3 (C), and PARP cleavage (D).

FIG. 32 shows the effect of treatment with 10 μM CX-4945 on VEGF expression in multiple myeloma cell lines.

FIG. 33 shows the effect of treatment with 10 μM CX-4945 on HIF-1α in multiple myeloma cell lines.

FIG. 34 illustrates the effects of increasing concentrations of CX-4945 on IL-6 secretion in U266 multiple myeloma cells.

FIG. 35 is a diagram illustrating the ability of CK2 to phosphorylate multiple substrates in the PIK3/Akt pathway.

FIG. 36 compares the ability of CX-4945 and various concentrations of staurosporine (STS) to inhibit phosphorylation of Akt-S129.

FIG. 37 shows the effect of 75 mg/kg bid CX-4945 on phosphorylation of Akt-S129, Akt-S473, and p21-T145 in mouse PBMCs.

FIG. 38 shows the results of a comet assay demonstrating the effect of CX-4945 on gemcitabine-induced DNA damage in A2780 ovarian cancer cells.

FIG. 39A shows the synergistic activity of gemcitabine and CX-4945 when administered at 60 mg/kg and 100 mg/kg, respectively in A2780 ovarian cancer xenografts.

FIG. 39B shows the synergistic activity of gemcitabine and CX-4945 on cancer cell apoptosis, as demonstrated by the increase in cleaved PARP (top panel). The bottom panel shows the synergistic activity of gemcitabine and CX-4945 in terms of percent tumor growth inhibition (TGI) and the medium number of days to reach the endpoint (TTE).

FIG. 40 is a diagram illustrating the relationship between EGFR and CK2 signaling.

FIG. 41 shows the effect of CX-4945 on epidermal growth factor (EGF)-stimulated CK2 activity in A431 (epidermoid carcinoma) and NCI-H2170 (lung cancer cells).

FIG. 42 shows the effect of 10 μM CX-4945 in combination with 50 μM erlotinib on the phosphorylation of Akt and rpS6 in NCI-H1650 and NCI-H1975 cells.

FIG. 43 illustrates the synergistic anti-tumor activity of CX-4945 and erlotinib in A431 epidermoid carcinoma cells.

### DETAILED DESCRIPTION

The present invention relates to biomarkers for predicting the sensitivity and/or monitoring the responsiveness of CK2-mediated diseases, including proliferative disorders and/or inflammatory disorders, to treatment with CK2 inhibitors.

As described herein, CK2 has been implicated in many type of cancerous cells (Table 1), and recent evidence suggests that CK2 exerts potent suppression of apoptosis in cancer cells by protecting regulatory proteins from caspase-mediated degradation.

TABLE 1
CK2 Link to Multiple Cancers.

| Cancer Type | Link with CK2 |
|---|---|
| Head & Neck | CK2 Overexpression, NF-κB, PI3K/Akt Activation, IL-6, |
| | EGFR/MAPK Act., Cdc37/Hsp90 |
| NSCLC | CK2 Overexpression, CK2 Amplification, PI3K/Akt Activation, |
| | EGFR/MAPK Act. |
| Breast | CK2 Overexpression, PI3K/Akt Activation, EGFR/MAPK Act., |
| | DNA Repair Defects, Cdc37/Hsp90 |
| Inflammatory Breast Cancer | CK2 Overexpression, PI3K/Akt Activation, IL-6, HIF-1α, |
| | EGFR/MAPK Act., DNA Repair Defects, Cdc37/Hsp90 |
| Prostate | CK2 Overexpression, PI3K/Akt Activation, Cdc37/Hsp90 |
| Pancreas | CK2 Overexpression, PI3K/Akt Activation, EGFR/MAPK |
| | Activation |
| Lymphomas (NHL, MCL) | CK2 Overexpression, PI3K/Akt Activation |
| Glioblastoma | CK2 Overexpression, PI3K/Akt Activation |
| SCC of Lung | CK2 Overexpression, PI3K/Akt Activation |
| Ovarian | CK2 Overexpression, PI3K/Akt Activation, DNA Repair Defects |
| Multiple Myeloma | CK2 Overexpression, PI3K/Akt Activation, IL-6, HIF-1α, |
| | Cdc37/Hsp90 |
| Acute Myeloid Leukemia | CK2 Overexpression, PI3K/Akt Activation |
| Colorectal | CK2 Overexpression, PI3K/Akt Activation |
| Thyroid | CK2 Overexpression, Akt activation, IL-6, IL-8 |

As described herein, the present inventors demonstrate that the mRNA expression and/or polypeptide levels of CK2α, CK2α′, IL-6, IL-8, VEGF, and HIF-1α and the phosphorylation levels of Akt, p21, NF-κB, STAT3, or JAK2, either alone or relative to total Akt, p21, NF-κB, STAT3, or JAK2, respectively, can be used as biomarkers to assess or predict the sensitivity, or lack of sensitivity, and/or monitor the responsiveness of a subject or system to treatment with a CK2 inhibitor.

### Interleukin-6

As shown in Examples 1-3, the use of a CK2 inhibitor, CX-4945, significantly reduces the concentration of IL-6 in inflammatory breast cancer (IBC) and prostate cancer patients. In addition, Example 11 shows that treatment with CX-4945 in U266 multiple myeloma cells reduces the production of IL-6. The present application also demonstrates that inhibitors of CK2 can inhibit the secretion of IL-6 by IBC cells SUM-149 PT in cell culture (IC₅₀˜2.5 μM) and in vivo. In a xenograft model, administration of CX-4945 caused a 40-60% (depending on tumor size) reduction in human IL-6 plasma levels after 50 mg/kg BID PO×3 d, 75 mg/kg PO daily or BID×8 d dosing. It was also discovered that aggressive (i.e. >1 g in weight) SUM-149PT xenografts produced significantly higher levels of IL-6 per g of tumor mass than the non-aggressive tumors (i.e. <1 g in weight).

IL-6 is an important cytokine in cancer biology, and is linked with CK2 activity in a variety of cancers, including head & neck cancer, inflammatory breast cancer, multiple myeloma, and thyroid cancer (Table 1). In multiple myeloma, IL-6 is predominantly produced in a paracrine fashion by multiple myeloma cells and bone marrow stromal cells (BMSCs). Under normal circumstances, IL-6 causes B-cell differentiation, but in multiple myeloma, it causes proliferation and inhibits apoptosis of myeloma cells. The interactions between multiple myeloma cells and BMSCs augment its secretion via nuclear factor-κB (NF-κB)-dependent pathways.

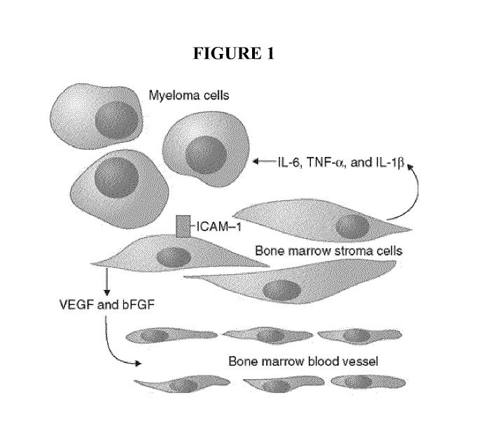

IL-6 has additional downstream effects on multiple myeloma cells. First, it promotes cell proliferation and survival via the RAS-MAPK pathway and JAK-STAT pathways, respectively. In addition, IL-6 prevents dexamethasone-induced apoptosis via the PI3K-AKT pathway and blocks differentiation of monocytes to dendritic cells, thus impairing host immune functions. Moreover, IL-6 induces VEGF (vascular endothelial growth factor) secretion, which promotes angiogenesis, stimulates growth and migration of multiple myeloma cells, further augments IL-6 secretion, and prevents antigen presentation by dendritic cells. See FIG. 1.

The present application presents data demonstrating that treatment with a CK2 inhibitor reduces IL-6 levels, and that IL-6 secretion and activity is a prominent hallmark of CK2-mediated diseases. Accordingly, in one embodiment, the invention provides a method for predicting the sensitivity of a CK2-mediated disease, such as a proliferative disorder and/or an inflammatory disorder, in a subject to treatment with a CK2 inhibitor, comprising determining the level of IL-6 mRNA expression and/or IL-6 polypeptide in a biological sample derived from the subject, wherein an increase in the level of IL-6 mRNA expression and/or IL-6 polypeptide relative to control is predictive of the sensitivity of the proliferative and/or inflammatory disorder to treatment with a CK2 inhibitor.

As used herein, the phrase the “level of a polypeptide” is used interchangeably with “protein expression levels” to refers to the process by which a nucleic acid sequence undergoes successful transcription and translation such that detectable levels of the amino acid sequence or protein are expressed and quantitated.

In another aspect, the invention provides a method for selecting subjects suffering from a CK2-mediated disease, such as a proliferative disorder and/or an inflammatory disorder, for treatment with a CK2 inhibitor, comprising predicting the sensitivity of the proliferative disorder and/or inflammatory disorder to treatment with a CK2 inhibitor in each subject by the foregoing method, and selecting those subjects showing an increased level of IL-6 mRNA expression and/or IL-6 polypeptide relative to control for treatment with a CK2 inhibitor.

In another aspect, the invention provides a method for treating a CK2-mediated disease, such as a proliferative disorder and/or an inflammatory disorder, in a subject in need thereof, comprising determining the level of IL-6 mRNA expression and/or IL-6 polypeptide in a biological sample derived from the subject by the method described above, and treating the subject with a CK2 inhibitor if the level of IL-6 mRNA expression and/or IL-6 polypeptide is elevated.

In some embodiments, the methods described above comprise determining the level of IL-6 mRNA expression relative to control. In other embodiments, the methods comprise determining the level of IL-6 polypeptide relative to control. In further embodiments, the methods comprise determining the level of IL-6 mRNA expression and the level of IL-6 polypeptide relative to control.

In a further aspect, the invention provides a method for monitoring of the responsiveness of a CK2-mediated disease in a subject to treatment with a CK2 inhibitor, comprising: (a) determining the level of IL-6 mRNA expression and/or IL-6 polypeptide in a first biological sample derived from the subject prior to treatment with a CK2 inhibitor; (b) determining the level of IL-6 mRNA expression and/or IL-6 polypeptide in at least a second biological sample derived from the subject subsequent to treatment with a CK2 inhibitor; and (c) comparing the level of IL-6 mRNA expression and/or IL-6 polypeptide in the second biological sample with the level of IL-6 mRNA expression and/or IL-6 polypeptide in the first biological sample; wherein a decrease in the level of IL-6 mRNA expression and/or IL-6 polypeptide in the second biological sample compared to the first biological sample is indicative of a positive response to treatment of the CK2-mediated disease to treatment with a CK2 inhibitor.

In some such embodiments, the level of IL-6 following treatment is decreased, indicating treatment with a CK2 inhibitor is effective to treat the CK2-mediated disease. In other embodiments, the rate of increase in IL-6 following treatment is reduced relative to an untreated control, indicating treatment with a CK2 inhibitor is effective to treat the CK2-mediated disease. In further embodiments, the level and/or the rate of increase in IL-6 is increased relative to control, indicating treatment is ineffective.

In a further aspect, the invention provides a method for identifying a compound useful for the treatment of a CK2-mediated proliferative disorder and/or inflammatory disorder, comprising: (a) analyzing the level of IL-6 mRNA expression and/or IL-6 polypeptide in a subject prior to treatment with the compound; and (b) analyzing the level of IL-6 mRNA expression and/or IL-6 polypeptide in a subject subsequent to treatment with the compound; wherein a decrease in the expression level of IL-6 mRNA expression and/or IL-6 polypeptide is indicative of drug efficacy.

In frequent embodiments related to IL-6 mRNA and/or polypeptide levels, the proliferative disorder comprises cancer or malignancy. In an exemplary embodiment, the cancer or malignancy is selected from breast cancer, inflammatory breast cancer (IBC), pancreatic cancer, prostate cancer, and multiple myeloma.

### Interleukin-8

The present application presents data demonstrating that treatment with a CK2 inhibitor reduces IL-8 levels, and that IL-8 secretion and activity is a prominent hallmark of CK2-mediated diseases. Accordingly, in another aspect, the invention provides a method for predicting the sensitivity of a CK2-mediated disease, such as a proliferative disorder and/or an inflammatory disorder, in a subject to treatment with a CK2 inhibitor, comprising determining the level of IL-8 mRNA expression and/or IL-8 polypeptide in a biological sample derived from the subject, wherein an increase in the level of IL-8 mRNA expression and/or IL-8 polypeptide relative to control is predictive of the sensitivity of the proliferative and/or inflammatory disorder to treatment with a CK2 inhibitor.

In another aspect, the invention provides a method for selecting subjects suffering from a CK2-mediated disease, such as a proliferative disorder and/or an inflammatory disorder, for treatment with a CK2 inhibitor, comprising predicting the sensitivity of the proliferative disorder and/or inflammatory disorder to treatment with a CK2 inhibitor in each subject by the foregoing method, and selecting those subjects showing an increased level of IL-8 mRNA expression and/or IL-8 polypeptide relative to control for treatment with a CK2 inhibitor.

In another aspect, the invention provides a method for treating a CK2-mediated disease, such as a proliferative disorder and/or an inflammatory disorder, in a subject in need thereof, comprising determining the level of IL-8 mRNA expression and/or IL-8 polypeptide in a biological sample derived from the subject by the method described above, and treating the subject with a CK2 inhibitor if the level of IL-8 mRNA expression and/or IL-8 polypeptide is elevated.

In some embodiments, the methods described above comprise determining the level of IL-8 mRNA expression relative to control. In other embodiments, the methods comprise determining the level of IL-8 polypeptide relative to control. In further embodiments, the methods comprise determining the level of IL-8 mRNA expression and the level of IL-8 polypeptide relative to control.

In a further aspect, the invention provides a method for monitoring of the responsiveness of a CK2-mediated disease in a subject to treatment with a CK2 inhibitor, comprising: (a) determining the level of IL-8 mRNA expression and/or IL-8 polypeptide in a first biological sample derived from the subject prior to treatment with a CK2 inhibitor; (b) determining the level of IL-8 mRNA expression and/or IL-8 polypeptide in at least a second biological sample derived from the subject subsequent to treatment with a CK2 inhibitor; and (c) comparing the level of IL-8 mRNA expression and/or IL-8 polypeptide in the second biological sample with the level of IL-8 mRNA expression and/or IL-8 polypeptide in the first biological sample; wherein a decrease in the level of IL-8 mRNA expression and/or IL-8 polypeptide in the second biological sample compared to the first biological sample is indicative of a positive response to treatment of the CK2-mediated disease to treatment with a CK2 inhibitor.

In some such embodiments, the level of IL-8 following treatment is decreased, indicating treatment with a CK2 inhibitor is effective to treat the CK2-mediated disease. In other embodiments, the rate of increase in IL-8 following treatment is reduced relative to an untreated control, indicating treatment with a CK2 inhibitor is effective to treat the CK2-mediated disease. In further embodiments, the level and/or the rate of increase in IL-8 is increased relative to control, indicating treatment is ineffective.

In another aspect, the invention provides a method for identifying a compound useful for the treatment of a CK2-mediated proliferative disorder and/or inflammatory disorder, comprising: (a) analyzing the level of IL-8 mRNA expression and/or IL-8 polypeptide in a subject prior to treatment with the compound; and (b) analyzing the level of IL-8 mRNA expression and/or IL-8 polypeptide in a subject subsequent to treatment with the compound; wherein a decrease in the expression level of IL-8 mRNA expression and/or IL-8 polypeptide is indicative of drug efficacy.

In frequent embodiments related to IL-8 mRNA and/or polypeptide levels, the proliferative disorder comprises cancer or malignancy. In an exemplary embodiment, the cancer or malignancy is selected from breast cancer, inflammatory breast cancer (IBC), pancreatic cancer, prostate cancer, and multiple myeloma.

### CK2α and CK2α′

The present application presents data demonstrating that elevated CK2α and/or CK2α′ expression is a prominent hallmark of CK2-mediated diseases (See, e.g., Examples 6 and 9, showing increased levels of CK2α subunit expression in breast cancer and multiple myeloma, respectively). Accordingly, in some embodiments, the biomarker comprises the mRNA expression level and/or polypeptide levels of the CK2α and/or the CK2α′ subunit, or both.

Thus in one aspect, the invention provides a method for predicting the sensitivity of a CK2-mediated disease, such as a proliferative disorder and/or an inflammatory disorder, in a subject to treatment with a CK2 inhibitor, comprising:

(i) determining the level of CK2α mRNA expression and/or CK2α polypeptide in a biological sample derived from the subject, wherein an increase in the level of CK2α mRNA expression and/or CK2α polypeptide relative to control is predictive of the sensitivity of the CK2-mediated disease to treatment with a CK2 inhibitor; and/or

(ii) determining the level of CK2α′ mRNA expression and/or CK2α′ polypeptide in a biological sample derived from the subject, wherein an increase in the level of CK2α′ mRNA expression and/or CK2α′ polypeptide relative to control is predictive of the sensitivity of the CK2-mediated disease to treatment with a CK2 inhibitor.

In frequent embodiments, the CK2-mediated disease is a proliferative disorder and/or an inflammatory disorder. In a specific embodiment, the CK2-mediated disease is selected from breast cancer, inflammatory breast cancer (IBC), and multiple myeloma.

In some embodiments, the methods described above comprise determining the level of CK2α mRNA expression relative to control. In other embodiments, the methods comprise determining the level of CK2α polypeptide relative to control. In further embodiments, the methods comprise determining the level of CK2α mRNA expression and the level of CK2α polypeptide relative to control.

In some embodiments, the methods described above comprise determining the level of CK2α′ mRNA expression relative to control. In other embodiments, the methods comprise determining the level of CK2α′ polypeptide relative to control. In further embodiments, the methods comprise determining the level of CK2α′ mRNA expression and the level of CK2α′ polypeptide relative to control.

In another aspect, the invention provides a method for selecting subjects suffering from a CK2-mediated disease, such as a proliferative disorder and/or an inflammatory disorder, for treatment with a CK2 inhibitor, comprising predicting the sensitivity of the proliferative disorder and/or inflammatory disorder to treatment with a CK2 inhibitor in each subject by the method above, and selecting those subjects showing an increased level of CK2α mRNA expression and/or CK2α polypeptide for treatment with a CK2 inhibitor.

In a further aspect, the invention provides a method for selecting subjects suffering from a CK2-mediated disease, such as a proliferative disorder and/or an inflammatory disorder, for treatment with a CK2 inhibitor, comprising predicting the sensitivity of the proliferative disorder and/or inflammatory disorder to treatment with a CK2 inhibitor in each subject by the method above, and selecting those subjects showing an increased level of CK2α′ mRNA expression and/or CK2α′ polypeptide for treatment with a CK2 inhibitor.

In frequent embodiments, the CK2-mediated disease is a proliferative disorder and/or an inflammatory disorder. In a specific embodiment, the CK2-mediated disease is selected from breast cancer, inflammatory breast cancer (IBC), and multiple myeloma.

In a further aspect, the invention provides a method for treating a CK2-mediated disease, such as a proliferative disorder and/or an inflammatory disorder, in a subject in need thereof, comprising:

(i) determining the level of CK2α mRNA expression and/or CK2α polypeptide in a biological sample derived from the subject by the method of claim 1, and treating the subject with a CK2 inhibitor if the level of CK2α mRNA expression and/or CK2α polypeptide is elevated; and/or

(ii) determining the level of CK2α′ mRNA expression and/or CK2α′ polypeptide in a biological sample derived from the subject by the method above, and treating the subject with a CK2 inhibitor if the level of CK2α′ mRNA expression and/or CK2α′ polypeptide is elevated.

The invention also provides a method for predicting the sensitivity of a subject to treatment with a CK2 inhibitor, comprising:

(i) determining the level of CK2α mRNA expression and/or CK2α polypeptide in a biological sample derived from the subject, wherein an increase in the level of CK2α mRNA expression and/or CK2α polypeptide relative to control is predictive of the sensitivity of the subject to treatment with a CK2 inhibitor; and/or

(ii) determining the level of CK2α′ mRNA expression and/or CK2α′ polypeptide in a biological sample derived from the subject, wherein an increase in the level of CK2α′ mRNA expression and/or CK2α′ polypeptide relative to control is predictive of the sensitivity of the subject to treatment with a CK2 inhibitor.

In another aspect, the invention provides a method to predict the response of a subject to treatment with a CK2 inhibitor, comprising determining the level of CK2α mRNA expression and/or CK2α polypeptide in a biological sample derived from the subject, wherein an increased level of CK2α mRNA expression and/or CK2α polypeptide relative to control is predictive of responsiveness to a CK2 inhibitor.

In a further aspect, the invention provides a method to predict the response of a subject to treatment with a CK2 inhibitor, comprising determining the level of CK2α′ mRNA expression and/or CK2α′ polypeptide in a biological sample derived from the subject, wherein an increased level of CK2α′ mRNA expression and/or CK2α′ polypeptide relative to control is predictive of responsiveness to a CK2 inhibitor.

In a further aspect, the invention provides a method for monitoring of the responsiveness of a CK2-mediated disease in a subject to treatment with a CK2 inhibitor, comprising: (a) determining the level of CK2α mRNA expression and/or CK2α polypeptide in a first biological sample derived from the subject prior to treatment with a CK2 inhibitor; (b) determining the level of CK2α mRNA expression and/or CK2α polypeptide in at least a second biological sample derived from the subject subsequent to treatment with a CK2 inhibitor; and (c) comparing the level of CK2α mRNA expression and/or CK2α polypeptide in the second biological sample with the level of CK2α mRNA expression and/or CK2α polypeptide in the first biological sample; wherein a decrease in the level of CK2α mRNA expression and/or CK2α polypeptide in the second biological sample compared to the first biological sample is indicative of a positive response to treatment of the CK2-mediated disease to treatment with a CK2 inhibitor.

In a further aspect, the invention provides a method for monitoring of the responsiveness of a CK2-mediated disease in a subject to treatment with a CK2 inhibitor, comprising: (a) determining the level of CK2α′ mRNA expression and/or CK2α′ polypeptide in a first biological sample derived from the subject prior to treatment with a CK2 inhibitor; (b) determining the level of CK2α′ mRNA expression and/or CK2α′ polypeptide in at least a second biological sample derived from the subject subsequent to treatment with a CK2 inhibitor; and (c) comparing the level of CK2α′ mRNA expression and/or CK2α′ polypeptide in the second biological sample with the level of CK2α′ mRNA expression and/or CK2α′ polypeptide in the first biological sample; wherein a decrease in the level of CK2α′ mRNA expression and/or CK2α′ polypeptide in the second biological sample compared to the first biological sample is indicative of a positive response to treatment of the CK2-mediated disease to treatment with a CK2 inhibitor.

In frequent embodiments related to CK2α and CK2α′ mRNA and/or polypeptide levels, the proliferative disorder comprises cancer or malignancy. In an exemplary embodiment, the cancer or malignancy is selected from breast cancer, inflammatory breast cancer (IBC), pancreatic cancer, prostate cancer, and multiple myeloma.

### Akt-S129 Phosphorylation

As shown in Examples 1, 5-8, 11-12, and 14, treatment with a CK2 inhibitor reduced the phosphorylation of Akt S129 in various cell lines, including breast cancer, pancreatic cancer, and multiple myeloma. As described herein, Akt-S129 is a CK2 specific biomarker, as CK2 phosphorylates and upregulates Akt/PKB. Thus, in certain aspects of the invention, the methods require assessing the phosphorylation status of Akt at Serine 129 in a biological sample, system or subject. In the methods described herein, the phosphorylation status of Akt may be determined by assessing the level of p-Akt S129 polypeptide alone (i.e., the absolute value). In some such embodiments, the level of p-Akt S129 polypeptide may be determined relative to a suitable control, such as a corresponding sample from a normal subject. In other embodiments, the normalized level of p-Akt S129 may be determined by assessing the level of p-Akt S129 polypeptide relative to total Akt, wherein the relative levels may sometimes be expressed as a percent or ratio of p-Akt S129 to total Akt. In some such embodiments, the corresponding control will be the normalized level of p-Akt S129 polypeptide to total Akt in a normal control.

In certain aspects of the invention, the methods require assessing the relationship between the mRNA and/or polypeptide levels of CK2α and/or CK2α′ and the phosphorylation status of p-Akt S129.

In one such aspect, the invention provides a method for predicting the sensitivity of a CK2-mediated disease, such as a proliferative disorder and/or an inflammatory disorder, in a subject to treatment with a CK2 inhibitor, comprising: (a) determining the level of CK2α′ mRNA expression and/or CK2α′ polypeptide in a biological sample derived from the subject; and (b) determining the level of phosphorylated Akt S129 (p-Akt S129) polypeptide in a biological sample derived from the subject; wherein a positive correlation between the level of CK2α′ mRNA expression and/or CK2α′ polypeptide and the level of p-Akt S129 polypeptide is predictive of sensitivity of the CK2-mediated disease to treatment with a CK2 inhibitor.

In another such aspect, the invention provides a method for selecting subjects suffering from a CK2-mediated disease, such as a proliferative disorder and/or an inflammatory disorder, for treatment with a CK2 inhibitor, comprising predicting the sensitivity of the CK2-mediated disease to treatment with a CK2 inhibitor in each subject by the method above, and selecting those subjects showing a positive correlation between the level of CK2α′ mRNA expression and/or CK2α′ polypeptide and the level of p-Akt S129 polypeptide.

In a further such aspect, the invention provides a method for treating a CK2-mediated disease, such as a proliferative disorder and/or an inflammatory disorder, in a subject in need thereof, comprising determining the level of CK2α′ mRNA expression and/or CK2α′ polypeptide in a biological sample derived from the subject and determining the level of p-Akt S129 polypeptide in a biological sample derived from the subject by the method of above, and treating the subject with a CK2 inhibitor if there is a positive correlation between the level of CK2α′ mRNA expression and/or CK2α′ polypeptide and the level of p-Akt S129 polypeptide.

In another aspect, the invention provides a method for predicting the sensitivity of a CK2-mediated disease, such as a proliferative disorder and/or an inflammatory disorder, in a subject to treatment with a CK2 inhibitor, comprising: (a) determining the level of CK2α mRNA expression and/or CK2α polypeptide in a biological sample derived from the subject; and (b) determining the level of phosphorylated Akt S129 (p-Akt S129) polypeptide in a biological sample derived from the subject; wherein a positive correlation between the level of CK2α mRNA expression and/or CK2α polypeptide and the level of p-Akt S129 polypeptide is predictive of sensitivity of the CK2-mediated disease to treatment with a CK2 inhibitor.

In another such aspect, the invention provides a method for selecting subjects suffering from a CK2-mediated disease, such as a proliferative disorder and/or an inflammatory disorder, for treatment with a CK2 inhibitor, comprising predicting the sensitivity of the proliferative disorder and/or inflammatory disorder to treatment with a CK2 inhibitor in each subject by the method above, and selecting those subjects showing a positive correlation between the level of CK2α mRNA expression and/or CK2α polypeptide and the level of p-Akt S129 polypeptide.

In a further such aspect, the invention provides a method for treating a CK2-mediated disease, such as a proliferative disorder and/or an inflammatory disorder, in a subject in need thereof, comprising determining the level of CK2α mRNA expression and/or CK2α polypeptide in a biological sample derived from the subject and determining the level of p-Akt S129 polypeptide in a biological sample derived from the subject by the method of above, and treating the subject with a CK2 inhibitor if there is a positive correlation between the level of CK2α mRNA expression and/or CK2α polypeptide and the level of p-Akt S129 polypeptide.

In another aspect, the invention provides a method for predicting the sensitivity of a CK2-mediated disease, such as a proliferative disorder and/or an inflammatory disorder, in a subject to treatment with a CK2 inhibitor, comprising: (a) determining the level of CK2α′ mRNA expression and/or CK2α′ polypeptide in a biological sample derived from the subject; and (b) determining the level of phosphorylated Akt S129 (p-Akt S129) polypeptide relative to the level of total Akt polypeptide in a biological sample derived from the subject; wherein a positive correlation between the level of CK2α′ mRNA expression and/or CK2α′ polypeptide and the normalized level of p-Akt S129 polypeptide is predictive of sensitivity of the CK2-mediated disease to treatment with a CK2 inhibitor.

In another such aspect, the invention provides a method for selecting subjects suffering from a CK2-mediated disease, such as a proliferative disorder and/or an inflammatory disorder, for treatment with a CK2 inhibitor, comprising predicting the sensitivity of the CK2-mediated disease to treatment with a CK2 inhibitor in each subject by the method above, and selecting those subjects showing a positive correlation between the level of CK2α′ mRNA expression and/or CK2α′ polypeptide and the normalized level of p-Akt S129 polypeptide.

In a further such aspect, the invention provides a method for treating a CK2-mediated disease, such as a proliferative disorder and/or an inflammatory disorder, in a subject in need thereof, comprising determining the level of CK2α′ mRNA expression and/or CK2α′ polypeptide in a biological sample derived from the subject and determining the level of p-Akt S129 polypeptide relative to the level of total Akt polypeptide in a biological sample derived from the subject by the method of above, and treating the subject with a CK2 inhibitor if there is a positive correlation between the level of CK2α′ mRNA expression and/or CK2α′ polypeptide and the normalized level of p-Akt S129 polypeptide.

In other aspects of the invention, the methods require determining the level of p-Akt S129 polypeptide in a system or subject. In some embodiments, the level of p-Akt S129 polypeptide is determined relative to total Akt polypeptide, to provide a normalized level of p-Akt S129 polypeptide. In other embodiments, the level of p-Akt S129 polypeptide alone is determined. Both the absolute and the normalized levels of p-Akt S129 polypeptide may be compared to the corresponding absolute or normalized controls derived from a normal system or subject.

Thus, in one aspect, the invention provides a method for predicting the sensitivity of a proliferative disorder and/or an inflammatory disorder in a subject to treatment with a CK2 inhibitor, comprising determining the level of p-Akt S129 polypeptide in a biological sample derived from the subject, wherein an increase in the level of p-Akt S129 polypeptide relative to control is predictive of the sensitivity of the proliferative and/or inflammatory disorder to treatment with a CK2 inhibitor.

In another aspect, the invention provides a method for selecting subjects suffering from a proliferative disorder and/or an inflammatory disorder for treatment with a CK2 inhibitor, comprising predicting the sensitivity of the proliferative disorder and/or inflammatory disorder to treatment with a CK2 inhibitor in each subject by the method above, and selecting those subjects showing an increased level of p-Akt S129 polypeptide for treatment with a CK2 inhibitor.

In a further aspect, the invention provides method for treating a proliferative disorder and/or an inflammatory disorder in a subject in need thereof, comprising determining the level of p-Akt S129 polypeptide in a biological sample derived from the subject by the method above, and treating the subject with a CK2 inhibitor if the level of p-Akt S129 polypeptide is elevated.

In another aspect, provides a method for monitoring of the responsiveness of a CK2-mediated disease in a subject to treatment with a CK2 inhibitor, comprising: (a) determining the level of p-Akt S129 polypeptide in a first biological sample derived from the subject prior to treatment with a CK2 inhibitor; (b) determining the level of p-Akt S129 polypeptide in at least a second biological sample derived from the subject subsequent to treatment with a CK2 inhibitor; and (c) comparing the level of p-Akt S129 polypeptide in the second biological sample with the level of p-Akt S129 polypeptide in the first biological sample; wherein a decrease in the level of p-Akt S129 polypeptide in the second biological sample compared to the first biological sample is indicative of a positive response to treatment with the CK2 inhibitor.

In another aspect, the invention provides a method to predict the response of a subject to treatment with a CK2 inhibitor, comprising determining the level of p-Akt S129 polypeptide in a biological sample derived from the subject, wherein an increased level of p-Akt S129 polypeptide relative to control is predictive of responsiveness to a CK2 inhibitor.

In further embodiments, the normalized level of p-Akt S129 polypeptide is used as a biomarker. The normalized level of p-Akt S129 polypeptide can be determined by assessing the level of p-Akt S129 polypeptide relative to total Akt polypeptide in a sample or subject.

In one such aspect, the invention provides a method for predicting the sensitivity of a proliferative disorder and/or an inflammatory disorder in a subject to treatment with a CK2 inhibitor, comprising determining the level of p-Akt S129 polypeptide relative to the level of total Akt polypeptide in a biological sample derived from the subject, wherein an increase in the normalized level of p-Akt S129 polypeptide relative to the corresponding control is predictive of the sensitivity of the proliferative and/or inflammatory disorder to treatment with a CK2 inhibitor.

In another such aspect, the invention provides a method for selecting subjects suffering from a proliferative disorder and/or an inflammatory disorder for treatment with a CK2 inhibitor, comprising predicting the sensitivity of the proliferative disorder and/or inflammatory disorder to treatment with a CK2 inhibitor in each subject by the foregoing method, and selecting those subjects showing an increased level of p-Akt S129 polypeptide relative to the level of total Akt polypeptide for treatment with a CK2 inhibitor.

In another such aspect, the invention provides a method for treating a proliferative disorder and/or an inflammatory disorder in a subject in need thereof, comprising determining the level of p-Akt S129 polypeptide relative to the level of total Akt polypeptide in a biological sample derived from the subject by the foregoing method, and treating the subject with a CK2 inhibitor if the level of p-Akt S129 polypeptide relative to the level of total Akt polypeptide is elevated.

In a further aspect, the invention provides a method for monitoring of the responsiveness of a CK2-mediated disease in a subject to treatment with a CK2 inhibitor, comprising: (a) determining the level of p-Akt S129 polypeptide relative to the level of total Akt polypeptide in a first biological sample derived from the subject prior to treatment with a CK2 inhibitor; (b) determining the level of p-Akt S129 polypeptide relative to the level of total Akt polypeptide in at least a second biological sample derived from the subject subsequent to treatment with a CK2 inhibitor; and (c) comparing the normalized level of p-Akt S129 polypeptide in the second biological sample with the normalized level of p-Akt S129 polypeptide in the first biological sample; wherein a decrease in the normalized level of p-Akt S129 polypeptide in the second biological sample compared to the first biological sample is indicative of a positive response to treatment with the CK2 inhibitor.

In another aspect, the invention provides a method for identifying a compound useful for the treatment of a CK2-mediated proliferative disorder and/or inflammatory disorder, comprising: (a) analyzing the level of p-Akt S129 polypeptide in a subject prior to treatment with the compound; and (b) analyzing the level of p-Akt S129 polypeptide in a subject subsequent to treatment with the compound; wherein a decrease in the level of p-Akt S129 polypeptide is indicative of drug efficacy.

In another aspect, the invention provides a method for identifying a compound useful for the treatment of a CK2-mediated proliferative disorder and/or inflammatory disorder, comprising: (a) analyzing the level of p-Akt S129 polypeptide relative to total Akt polypeptide level in a subject prior to treatment with the compound; and (b) analyzing the level of p-Akt S129 polypeptide relative to total Akt polypeptide level in a subject subsequent to treatment with the compound; wherein a decrease in the normalized level of p-Akt S129 polypeptide is indicative of drug efficacy.

In frequent embodiments related to p-Akt S129 polypeptide levels, the proliferative disorder comprises cancer or malignancy. In an exemplary embodiment, the cancer or malignancy is selected from breast cancer, inflammatory breast cancer (IBC), pancreatic cancer, prostate cancer, and multiple myeloma.

### Akt-S473 Phosphorylation

As shown in Examples 1, 8, 11-12, and 14, treatment with a CK2 inhibitor reduced the phosphorylation of Akt S473 in various cell lines, including breast cancer, pancreatic cancer, and multiple myeloma. Accordingly, in certain aspects of the invention, the methods require assessing the phosphorylation status of Akt at Serine 473 in a biological sample, system or subject. In the methods described herein, the phosphorylation status of Akt may be determined by assessing the level of p-Akt S473 polypeptide alone (i.e., the absolute value). In some such embodiments, the level of p-Akt S473 polypeptide may be determined relative to a suitable control, such as a corresponding sample from a normal subject. In other embodiments, the normalized level of p-Akt S473 may be determined by assessing the level of p-Akt S473 polypeptide relative to total Akt, wherein the relative levels may sometimes be expressed as a percent or ratio of p-Akt S473 to total Akt. In some such embodiments, the corresponding control will be the normalized level of p-Akt S473 polypeptide to total Akt in a normal control.

In one such aspect, the invention provides a method for monitoring of the responsiveness of a CK2-mediated disease in a subject to treatment with a CK2 inhibitor, comprising: (a) determining the level of p-Akt S473 polypeptide in a first biological sample derived from the subject prior to treatment with a CK2 inhibitor; (b) determining the level of p-Akt S473 polypeptide in at least a second biological sample derived from the subject subsequent to treatment with a CK2 inhibitor; and (c) comparing the level of p-Akt S473 polypeptide in the second biological sample with the level of p-Akt S473 polypeptide in the first biological sample; wherein a decrease in the level of p-Akt S473 polypeptide in the second biological sample compared to the first biological sample is indicative of a positive response to treatment with the CK2 inhibitor. In another such aspect, the invention provides a method for monitoring of the responsiveness of a CK2-mediated disease in a subject to treatment with a CK2 inhibitor, comprising: (a) determining the level of p-Akt S473 polypeptide relative to the level of total Akt polypeptide in a first biological sample derived from the subject prior to treatment with a CK2 inhibitor; (b) determining the level of p-Akt S473 polypeptide relative to the level of total Akt polypeptide in at least a second biological sample derived from the subject subsequent to treatment with a CK2 inhibitor; and (c) comparing the normalized level of p-Akt S473 polypeptide in the second biological sample with the normalized level of p-Akt S473 polypeptide in the first biological sample; wherein a decrease in the normalized level of p-Akt S473 polypeptide in the second biological sample compared to the first biological sample is indicative of a positive response to treatment with the CK2 inhibitor.

In another aspect, the invention provides a method for predicting the sensitivity of a CK2-mediated disorder, such as a proliferative disorder and/or an inflammatory disorder, in a subject to treatment with a CK2 inhibitor, comprising determining the level of p-Akt S473 polypeptide in a biological sample derived from the subject, wherein an increase in the level of p-Akt S473 polypeptide relative to control is predictive of the sensitivity of the proliferative and/or inflammatory disorder to treatment with a CK2 inhibitor.

In yet another aspect, the invention provides a method for predicting the sensitivity of a CK2-mediated disorder, such as a proliferative disorder and/or an inflammatory disorder, in a subject to treatment with a CK2 inhibitor, comprising determining the level of p-Akt S473 polypeptide relative to the level of total Akt polypeptide in a biological sample derived from the subject, wherein an increase in the normalized level of p-Akt S473 polypeptide relative to the corresponding control is predictive of the sensitivity of the CK2-mediated disorder to treatment with a CK2 inhibitor.

In another such aspect, the invention provides a method for selecting subjects suffering from a CK2-mediated disorder, such as a proliferative disorder and/or an inflammatory disorder, for treatment with a CK2 inhibitor, comprising predicting the sensitivity of the CK2-mediated disorder to treatment with a CK2 inhibitor in each subject by one of the foregoing methods, and selecting those subjects showing an increased level of p-Akt S473 polypeptide or an increase in the normalized level of p-Akt S473 polypeptide for treatment with a CK2 inhibitor.

In another aspect, the invention provides a method for treating a CK2-mediated disorder, such as a proliferative disorder and/or inflammatory disorder, in a subject in need thereof, comprising determining the level of p-Akt S473 polypeptide in a biological sample derived from the subject by one of the foregoing methods, and treating the subject with a CK2 inhibitor if the level of p-Akt S473 polypeptide is elevated.

In another aspect, the invention provides a method to predict the response of a subject to treatment with a CK2 inhibitor, comprising determining the level of p-Akt S473 polypeptide alone or relative to the level of total Akt polypeptide in a biological sample derived from the subject, wherein an increase in the absolute or normalized level of p-Akt S473 polypeptide relative to corresponding control is predictive of responsiveness to a CK2 inhibitor.

In a further aspect, the invention provides a method for identifying a compound useful for the treatment of a CK2-mediated disorder, such as a proliferative disorder and/or inflammatory disorder, comprising: (a) analyzing the level of p-Akt S473 polypeptide in a subject prior to treatment with the compound; and (b) analyzing the level of p-Akt S473 polypeptide in a subject subsequent to treatment with the compound; wherein a decrease in the level of p-Akt S473 polypeptide is indicative of drug efficacy.

In still another aspect, the invention provides a method for identifying a compound useful for the treatment of a CK2-mediated disorder, such as a proliferative disorder and/or inflammatory disorder, comprising: (a) analyzing the level of p-Akt S473 polypeptide relative to total Akt polypeptide in a subject prior to treatment with the compound; and (b) analyzing the level of p-Akt S473 polypeptide relative to total Akt polypeptide in a subject subsequent to treatment with the compound; wherein a decrease in the normalized level of p-Akt S473 polypeptide is indicative of drug efficacy.

In frequent embodiments related to p-Akt S473 polypeptide levels, the proliferative disorder comprises cancer or malignancy. In an exemplary embodiment, the cancer or malignancy is selected from breast cancer, inflammatory breast cancer (IBC), pancreatic cancer, prostate cancer, and multiple myeloma.

p21-T145 Phosphorylation

As shown in Examples 1 and 8, 11-12, and 14, treatment with a CK2 inhibitor reduced the phosphorylation of p21 T145 in various cell lines, including breast and pancreatic cancer cell lines. Accordingly, in other aspects of the invention, the methods require assessing the phosphorylation status of p21 at threonine 145 (p-p21 T145) in a biological sample, system or subject. In the methods described herein, the phosphorylation status of p21 may be determined by assessing the level of p-p21 T145 polypeptide alone (i.e., the absolute value). In some such embodiments, the level of p-p21 T145 polypeptide may be determined relative to a suitable control, such as a corresponding sample from a normal subject. In other embodiments, the normalized level of p-p21 T145 may be determined by assessing the level of p-p21 T145 polypeptide relative to total p21, wherein the relative levels may sometimes be expressed as a percent or ratio of p-p21 T145 to total p21. In some such embodiments, the corresponding control will be the normalized level of p-p21 T145 polypeptide to total p21 in a normal control.

In one such aspect, the invention provides a method for monitoring of the responsiveness of a CK2-mediated disease in a subject to treatment with a CK2 inhibitor, comprising: (a) determining the level of p-p21 T145 polypeptide in a first biological sample derived from the subject prior to treatment with a CK2 inhibitor; (b) determining the level of p-p21 T145 polypeptide in at least a second biological sample derived from the subject subsequent to treatment with a CK2 inhibitor; and (c) comparing the level of p-p21 T145 polypeptide in the second biological sample with the level of p-p21 T145 polypeptide in the first biological sample; wherein a decrease in the level of p-p21 T145 polypeptide in the second biological sample compared to the first biological sample is indicative of a positive response to treatment with the CK2 inhibitor.

In another such aspect, the invention provides a method for monitoring of the responsiveness of a CK2-mediated disease in a subject to treatment with a CK2 inhibitor, comprising: (a) determining the level of p-p21 T145 polypeptide relative to the level of total p21 polypeptide in a first biological sample derived from the subject prior to treatment with a CK2 inhibitor; (b) determining the level of p-p21 T145 polypeptide relative to the level of total p21 polypeptide in at least a second biological sample derived from the subject subsequent to treatment with a CK2 inhibitor; and (c) comparing the normalized level of p-p21 T145 polypeptide in the second biological sample with the normalized level of p-p21 T145 polypeptide in the first biological sample; wherein a decrease in the normalized level of p-p21 T145 polypeptide in the second biological sample compared to the first biological sample is indicative of a positive response to treatment with the CK2 inhibitor.

In a further aspect, the invention provides a method for predicting the sensitivity of a CK2-mediated disorder, such as a proliferative disorder and/or an inflammatory disorder, in a subject to treatment with a CK2 inhibitor, comprising determining the level of p-p21 T145 polypeptide in a biological sample derived from the subject, wherein an increase in the level of p-p21 T145 polypeptide relative to control is predictive of the sensitivity of the CK2-mediated disorder to treatment with a CK2 inhibitor.

In another aspect, the invention provides a method for predicting the sensitivity of a CK2-mediated disorder, such as a proliferative disorder and/or an inflammatory disorder, in a subject to treatment with a CK2 inhibitor, comprising determining the level of p-p21 T145 polypeptide relative to the level of total p21 polypeptide in a biological sample derived from the subject, wherein an increase in the normalized level of p-p21 T145 polypeptide relative to the corresponding control is predictive of the sensitivity of the CK2-mediated disorder to treatment with a CK2 inhibitor.

In yet another aspect, the invention provides method for selecting subjects suffering from a CK2-mediated disorder, such as a proliferative disorder and/or an inflammatory disorder, for treatment with a CK2 inhibitor, comprising predicting the sensitivity of the CK2-mediated disorder to treatment with a CK2 inhibitor in each subject by one of the foregoing methods, and selecting those subjects showing an increased level of p-p21 T145 polypeptide for treatment with a CK2 inhibitor.

In still another aspect, the invention provides method for selecting subjects suffering from a CK2-mediated disorder, such as a proliferative disorder and/or an inflammatory disorder for treatment with a CK2 inhibitor, comprising predicting the sensitivity of the CK2-mediated disorder to treatment with a CK2 inhibitor, and selecting those subjects showing an increase in the normalized level of p-p21 T145 polypeptide for treatment with a CK2 inhibitor.

In a further aspect, the invention provides a method to predict the response of a subject to treatment with a CK2 inhibitor, comprising determining the level of p-p21 T145 polypeptide in a biological sample derived from the subject, wherein an increased level of p-p21 T145 polypeptide relative to control is predictive of responsiveness to a CK2 inhibitor.

In another aspect, the invention provides a method for treating a CK2-mediated disorder, such as a proliferative disorder and/or an inflammatory disorder, in a subject in need thereof, comprising determining the level of p-p21 T145 polypeptide in a biological sample derived from the subject by one of the foregoing methods, and treating the subject with a CK2 inhibitor if the level of p-p21 T145 polypeptide is elevated.

In another aspect, the invention provides a method to predict the response of a subject to treatment with a CK2 inhibitor, comprising determining the level of p-p21 T145 polypeptide relative to the level of total p21 polypeptide in a biological sample derived from the subject, wherein an increased normalized level of p-p21 T145 polypeptide relative to corresponding control is predictive of responsiveness to a CK2 inhibitor.

In another aspect, the invention provides a method for identifying a compound useful for the treatment of a CK2-mediated proliferative disorder and/or inflammatory disorder, comprising: (a) analyzing the level of p-p21 T145 polypeptide relative to total p21 polypeptide in a subject prior to treatment with the compound; and (b) analyzing the level of p-p21 T145 polypeptide relative to total p21 polypeptide in a subject subsequent to treatment with the compound; wherein a decrease in the normalized level of p-p21 T145 polypeptide is indicative of drug efficacy.

In frequent embodiments related to p-p21 T145 polypeptide levels, the proliferative disorder comprises cancer or malignancy. In an exemplary embodiment, the cancer or malignancy is selected from breast cancer, inflammatory breast cancer (IBC), pancreatic cancer, and multiple myeloma.

### NF-κB S529 Phosphorylation

As shown in Example 11, treatment with a CK2 inhibitor reduced the phosphorylation of NF-κB S529 in various multiple myeloma cell lines. Accordingly, in certain aspects of the invention, the methods require assessing the phosphorylation status of NF-κB at Serine 529 in a biological sample, system or subject. In the methods described herein, the phosphorylation status of NF-κB may be determined by assessing the level of p-NF-κB S529 polypeptide alone (i.e., the absolute value). In some such embodiments, the level of p-NF-κB S529 polypeptide may be determined relative to a suitable control, such as a corresponding sample from a normal subject. In other embodiments, the normalized level of p-NF-κB S529 may be determined by assessing the level of p-NF-κB S529 polypeptide relative to total NF-κB, wherein the relative levels may sometimes be expressed as a percent or ratio of p-NF-κB S529 to total NF-κB. In some such embodiments, the corresponding control will be the normalized level of p-NF-κB S529 polypeptide to total NF-κB in a normal control.

In one such aspect, the invention provides a method for monitoring of the responsiveness of a CK2-mediated disease in a subject to treatment with a CK2 inhibitor, comprising: (a) determining the level of p-NF-κB S529 polypeptide in a first biological sample derived from the subject prior to treatment with a CK2 inhibitor; (b) determining the level of p-NF-κB S529 polypeptide in at least a second biological sample derived from the subject subsequent to treatment with a CK2 inhibitor; and (c) comparing the level of p-NF-κB S529 polypeptide in the second biological sample with the level of p-NF-κB S529 polypeptide in the first biological sample; wherein a decrease in the level of p-NF-κB S529 polypeptide in the second biological sample compared to the first biological sample is indicative of a positive response to treatment with the CK2 inhibitor.

In another such aspect, the invention provides a method for monitoring of the responsiveness of a CK2-mediated disease in a subject to treatment with a CK2 inhibitor, comprising: (a) determining the level of p-NF-κB S529 polypeptide relative to the level of total NF-κB polypeptide in a first biological sample derived from the subject prior to treatment with a CK2 inhibitor; (b) determining the level of p-NF-κB S529 polypeptide relative to the level of total NF-κB polypeptide in at least a second biological sample derived from the subject subsequent to treatment with a CK2 inhibitor; and (c) comparing the normalized level of p-NF-κB S529 polypeptide in the second biological sample with the normalized level of p-NF-κB S529 polypeptide in the first biological sample; wherein a decrease in the normalized level of p-NF-κB S529 polypeptide in the second biological sample compared to the first biological sample is indicative of a positive response to treatment with the CK2 inhibitor.

In another aspect, the invention provides a method for predicting the sensitivity of a CK2-mediated disorder, such as a proliferative disorder and/or an inflammatory disorder, in a subject to treatment with a CK2 inhibitor, comprising determining the level of p-NF-κB S529 polypeptide in a biological sample derived from the subject, wherein an increase in the level of p-NF-κB S529 polypeptide relative to control is predictive of the sensitivity of the proliferative and/or inflammatory disorder to treatment with a CK2 inhibitor.

In yet another aspect, the invention provides a method for predicting the sensitivity of a CK2-mediated disorder, such as a proliferative disorder and/or an inflammatory disorder, in a subject to treatment with a CK2 inhibitor, comprising determining the level of p-NF-κB S529 polypeptide relative to the level of total NF-κB polypeptide in a biological sample derived from the subject, wherein an increase in the normalized level of p-NF-κB S529 polypeptide relative to the corresponding control is predictive of the sensitivity of the CK2-mediated disorder to treatment with a CK2 inhibitor.

In another such aspect, the invention provides a method for selecting subjects suffering from a CK2-mediated disorder, such as a proliferative disorder and/or an inflammatory disorder, for treatment with a CK2 inhibitor, comprising predicting the sensitivity of the CK2-mediated disorder to treatment with a CK2 inhibitor in each subject by one of the foregoing methods, and selecting those subjects showing an increased level of p-NF-κB S529 polypeptide or an increase in the normalized level of p-NF-κB S529 polypeptide for treatment with a CK2 inhibitor.

In another aspect, the invention provides a method for treating a CK2-mediated disorder, such as a proliferative disorder and/or inflammatory disorder, in a subject in need thereof, comprising determining the level of p-NF-κB S529 polypeptide in a biological sample derived from the subject by one of the foregoing methods, and treating the subject with a CK2 inhibitor if the level of p-NF-κB S529 polypeptide is elevated.

In another aspect, the invention provides a method to predict the response of a subject to treatment with a CK2 inhibitor, comprising determining the level of p-NF-κB S529 polypeptide alone or relative to the level of total NF-κB polypeptide in a biological sample derived from the subject, wherein an increase in the absolute or normalized level of p-NF-κB S529 polypeptide relative to corresponding control is predictive of responsiveness to a CK2 inhibitor.

In a further aspect, the invention provides a method for identifying a compound useful for the treatment of a CK2-mediated disorder, such as a proliferative disorder and/or inflammatory disorder, comprising: (a) analyzing the level of p-NF-κB S529 polypeptide in a subject prior to treatment with the compound; and (b) analyzing the level of p-NF-κB S529 polypeptide in a subject subsequent to treatment with the compound; wherein a decrease in the level of p-NF-κB S529 polypeptide is indicative of drug efficacy.

In still another aspect, the invention provides a method for identifying a compound useful for the treatment of a CK2-mediated disorder, such as a proliferative disorder and/or inflammatory disorder, comprising: (a) analyzing the level of p-NF-κB S529 polypeptide relative to total NF-κB polypeptide in a subject prior to treatment with the compound; and (b) analyzing the level of p-NF-κB S529 polypeptide relative to total NF-κB polypeptide in a subject subsequent to treatment with the compound; wherein a decrease in the normalized level of p-NF-κB S529 polypeptide is indicative of drug efficacy.

In frequent embodiments related to p-NF-κB S529 polypeptide levels, the proliferative disorder comprises cancer or malignancy. In an exemplary embodiment, the cancer or malignancy is multiple myeloma.

### STAT3-Y705 Phosphorylation

As shown in Example 11, treatment with a CK2 inhibitor reduced the phosphorylation of STAT3 Y705 in various multiple myeloma cell lines. Accordingly, in certain aspects of the invention, the methods require assessing the phosphorylation status of STAT3 at tyrosine 705 in a biological sample, system or subject. In the methods described herein, the phosphorylation status of STAT3 may be determined by assessing the level of p-STAT3 Y705 polypeptide alone (i.e., the absolute value). In some such embodiments, the level of p-STAT3 Y705 polypeptide may be determined relative to a suitable control, such as a corresponding sample from a normal subject. In other embodiments, the normalized level of p-STAT3 Y705 may be determined by assessing the level of p-STAT3 Y705 polypeptide relative to total STAT3, wherein the relative levels may sometimes be expressed as a percent or ratio of p-STAT3 Y705 to total STAT3. In some such embodiments, the corresponding control will be the normalized level of p-STAT3 Y705 polypeptide to total STAT3 in a normal control.

In one such aspect, the invention provides a method for monitoring of the responsiveness of a CK2-mediated disease in a subject to treatment with a CK2 inhibitor, comprising: (a) determining the level of p-STAT3 Y705 polypeptide in a first biological sample derived from the subject prior to treatment with a CK2 inhibitor; (b) determining the level of p-STAT3 Y705 polypeptide in at least a second biological sample derived from the subject subsequent to treatment with a CK2 inhibitor; and (c) comparing the level of p-STAT3 Y705 polypeptide in the second biological sample with the level of p-STAT3 Y705 polypeptide in the first biological sample; wherein a decrease in the level of p-STAT3 Y705 polypeptide in the second biological sample compared to the first biological sample is indicative of a positive response to treatment with the CK2 inhibitor.

In another such aspect, the invention provides a method for monitoring of the responsiveness of a CK2-mediated disease in a subject to treatment with a CK2 inhibitor, comprising: (a) determining the level of p-STAT3 Y705 polypeptide relative to the level of total STAT3 polypeptide in a first biological sample derived from the subject prior to treatment with a CK2 inhibitor; (b) determining the level of p-STAT3 Y705 polypeptide relative to the level of total STAT3 polypeptide in at least a second biological sample derived from the subject subsequent to treatment with a CK2 inhibitor; and (c) comparing the normalized level of p-STAT3 Y705 polypeptide in the second biological sample with the normalized level of p-STAT3 Y705 polypeptide in the first biological sample; wherein a decrease in the normalized level of p-STAT3 Y705 polypeptide in the second biological sample compared to the first biological sample is indicative of a positive response to treatment with the CK2 inhibitor.

In another aspect, the invention provides a method for predicting the sensitivity of a CK2-mediated disorder, such as a proliferative disorder and/or an inflammatory disorder, in a subject to treatment with a CK2 inhibitor, comprising determining the level of p-STAT3 Y705 polypeptide in a biological sample derived from the subject, wherein an increase in the level of p-STAT3 Y705 polypeptide relative to control is predictive of the sensitivity of the proliferative and/or inflammatory disorder to treatment with a CK2 inhibitor.

In yet another aspect, the invention provides a method for predicting the sensitivity of a CK2-mediated disorder, such as a proliferative disorder and/or an inflammatory disorder, in a subject to treatment with a CK2 inhibitor, comprising determining the level of p-STAT3 Y705 polypeptide relative to the level of total STAT3 polypeptide in a biological sample derived from the subject, wherein an increase in the normalized level of p-STAT3 Y705 polypeptide relative to the corresponding control is predictive of the sensitivity of the CK2-mediated disorder to treatment with a CK2 inhibitor.

In another such aspect, the invention provides a method for selecting subjects suffering from a CK2-mediated disorder, such as a proliferative disorder and/or an inflammatory disorder, for treatment with a CK2 inhibitor, comprising predicting the sensitivity of the CK2-mediated disorder to treatment with a CK2 inhibitor in each subject by one of the foregoing methods, and selecting those subjects showing an increased level of p-STAT3 Y705 polypeptide or an increase in the normalized level of p-STAT3 Y705 polypeptide for treatment with a CK2 inhibitor.

In another aspect, the invention provides a method for treating a CK2-mediated disorder, such as a proliferative disorder and/or inflammatory disorder, in a subject in need thereof, comprising determining the level of p-STAT3 Y705 polypeptide in a biological sample derived from the subject by one of the foregoing methods, and treating the subject with a CK2 inhibitor if the level of p-STAT3 Y705 polypeptide is elevated.

In another aspect, the invention provides a method to predict the response of a subject to treatment with a CK2 inhibitor, comprising determining the level of p-STAT3 Y705 polypeptide alone or relative to the level of total STAT3 polypeptide in a biological sample derived from the subject, wherein an increase in the absolute or normalized level of p-STAT3 Y705 polypeptide relative to corresponding control is predictive of responsiveness to a CK2 inhibitor.

In a further aspect, the invention provides a method for identifying a compound useful for the treatment of a CK2-mediated disorder, such as a proliferative disorder and/or inflammatory disorder, comprising: (a) analyzing the level of p-STAT3 Y705 polypeptide in a subject prior to treatment with the compound; and (b) analyzing the level of p-STAT3 Y705 polypeptide in a subject subsequent to treatment with the compound; wherein a decrease in the level of p-STAT3 Y705 polypeptide is indicative of drug efficacy.

In still another aspect, the invention provides a method for identifying a compound useful for the treatment of a CK2-mediated disorder, such as a proliferative disorder and/or inflammatory disorder, comprising: (a) analyzing the level of p-STAT3 Y705 polypeptide relative to total STAT3 polypeptide in a subject prior to treatment with the compound; and (b) analyzing the level of p-STAT3 Y705 polypeptide relative to total STAT3 polypeptide in a subject subsequent to treatment with the compound; wherein a decrease in the normalized level of p-STAT3 Y705 polypeptide is indicative of drug efficacy.

In frequent embodiments related to p-STAT3 Y705 polypeptide levels, the proliferative disorder comprises cancer or malignancy. In an exemplary embodiment, the cancer or malignancy is multiple myeloma.

### JAK2-Y1007/1008 Phosphorylation

As shown in Example 11, treatment with a CK2 inhibitor reduced the phosphorylation of JAK2 Y1007/1008 in various multiple myeloma cell lines. Accordingly, in certain aspects of the invention, the methods require assessing the phosphorylation status of JAK2 at tyrosine residues 1007 and 1008 in a biological sample, system or subject. In the methods described herein, the phosphorylation status of STAT3 may be determined by assessing the level of p-JAK2 Y1007/1008 polypeptide alone (i.e., the absolute value). In some such embodiments, the level of p-JAK2 Y1007/1008 polypeptide may be determined relative to a suitable control, such as a corresponding sample from a normal subject. In other embodiments, the normalized level of p-JAK2 Y1007/1008 may be determined by assessing the level of p-JAK2 Y1007/1008 polypeptide relative to total JAK2, wherein the relative levels may sometimes be expressed as a percent or ratio of p-JAK2 Y1007/1008 to total JAK2. In some such embodiments, the corresponding control will be the normalized level of p-JAK2 Y1007/1008 polypeptide to total JAK2 in a normal control

In one such aspect, the invention provides a method for monitoring of the responsiveness of a CK2-mediated disease in a subject to treatment with a CK2 inhibitor, comprising: (a) determining the level of p-JAK2 Y1007/1008 polypeptide in a first biological sample derived from the subject prior to treatment with a CK2 inhibitor; (b) determining the level of p-JAK2 Y1007/1008 polypeptide in at least a second biological sample derived from the subject subsequent to treatment with a CK2 inhibitor; and (c) comparing the level of p-JAK2 Y1007/1008 polypeptide in the second biological sample with the level of p-JAK2 Y1007/1008 polypeptide in the first biological sample; wherein a decrease in the level of p-JAK2 Y1007/1008 polypeptide in the second biological sample compared to the first biological sample is indicative of a positive response to treatment with the CK2 inhibitor.

In another such aspect, the invention provides a method for monitoring of the responsiveness of a CK2-mediated disease in a subject to treatment with a CK2 inhibitor, comprising: (a) determining the level of p-JAK2 Y1007/1008 polypeptide relative to the level of total JAK2 polypeptide in a first biological sample derived from the subject prior to treatment with a CK2 inhibitor; (b) determining the level of p-JAK2 Y1007/1008 polypeptide relative to the level of total JAK2 polypeptide in at least a second biological sample derived from the subject subsequent to treatment with a CK2 inhibitor; and (c) comparing the normalized level of p-JAK2 Y1007/1008 polypeptide in the second biological sample with the normalized level of p-JAK2 Y1007/1008 polypeptide in the first biological sample; wherein a decrease in the normalized level of p-JAK2 Y1007/1008 polypeptide in the second biological sample compared to the first biological sample is indicative of a positive response to treatment with the CK2 inhibitor.

In another aspect, the invention provides a method for predicting the sensitivity of a CK2-mediated disorder, such as a proliferative disorder and/or an inflammatory disorder, in a subject to treatment with a CK2 inhibitor, comprising determining the level of p-JAK2 Y1007/1008 polypeptide in a biological sample derived from the subject, wherein an increase in the level of p-JAK2 Y1007/1008 polypeptide relative to control is predictive of the sensitivity of the proliferative and/or inflammatory disorder to treatment with a CK2 inhibitor.

In yet another aspect, the invention provides a method for predicting the sensitivity of a CK2-mediated disorder, such as a proliferative disorder and/or an inflammatory disorder, in a subject to treatment with a CK2 inhibitor, comprising determining the level of p-JAK2 Y1007/1008 polypeptide relative to the level of total JAK2 polypeptide in a biological sample derived from the subject, wherein an increase in the normalized level of p-JAK2 Y1007/1008 polypeptide relative to the corresponding control is predictive of the sensitivity of the CK2-mediated disorder to treatment with a CK2 inhibitor.

In another such aspect, the invention provides a method for selecting subjects suffering from a CK2-mediated disorder, such as a proliferative disorder and/or an inflammatory disorder, for treatment with a CK2 inhibitor, comprising predicting the sensitivity of the CK2-mediated disorder to treatment with a CK2 inhibitor in each subject by one of the foregoing methods, and selecting those subjects showing an increased level of p-JAK2 Y1007/1008 polypeptide or an increase in the normalized level of p-JAK2 Y1007/1008 polypeptide for treatment with a CK2 inhibitor.

In another aspect, the invention provides a method for treating a CK2-mediated disorder, such as a proliferative disorder and/or inflammatory disorder, in a subject in need thereof, comprising determining the level of p-JAK2 Y1007/1008 polypeptide in a biological sample derived from the subject by one of the foregoing methods, and treating the subject with a CK2 inhibitor if the level of p-JAK2 Y1007/1008 polypeptide is elevated.

In another aspect, the invention provides a method to predict the response of a subject to treatment with a CK2 inhibitor, comprising determining the level of p-JAK2 Y1007/1008 polypeptide alone or relative to the level of total JAK2 polypeptide in a biological sample derived from the subject, wherein an increase in the absolute or normalized level of p-JAK2 Y1007/1008 polypeptide relative to corresponding control is predictive of responsiveness to a CK2 inhibitor.

In a further aspect, the invention provides a method for identifying a compound useful for the treatment of a CK2-mediated disorder, such as a proliferative disorder and/or inflammatory disorder, comprising: (a) analyzing the level of p-JAK2-Y1007/1008 polypeptide in a subject prior to treatment with the compound; and (b) analyzing the level of p-JAK2-Y1007/1008 polypeptide in a subject subsequent to treatment with the compound; wherein a decrease in the level of p-JAK2-Y1007/1008 polypeptide is indicative of drug efficacy.

In still another aspect, the invention provides a method for identifying a compound useful for the treatment of a CK2-mediated disorder, such as a proliferative disorder and/or inflammatory disorder, comprising: (a) analyzing the level of p-JAK2-Y1007/1008 polypeptide relative to total JAK2 polypeptide in a subject prior to treatment with the compound; and (b) analyzing the level of p-JAK2-Y1007/1008 polypeptide relative to total JAK2 polypeptide in a subject subsequent to treatment with the compound; wherein a decrease in the normalized level of p-JAK2-Y1007/1008 polypeptide is indicative of drug efficacy.

In frequent embodiments related to p-JAK2 Y1007/1008 polypeptide levels, the proliferative disorder comprises cancer or malignancy. In an exemplary embodiment, the cancer or malignancy is multiple myeloma.

### Use of One or More Biomarkers for the Creation of Sample Profiles

In yet another aspect, the invention provides a method for predicting responders from non-responders for treatment of a CK2-mediated disease, such as a proliferative disorder and/or an inflammatory disorder, with a CK2 inhibitor, comprising: (a) determining the level of mRNA expression and/or polypeptide level of one or more biomarkers selected from CK2α, CK2α′, and IL-6, IL-8, VEGF, HIF-1α and/or the level of p-Akt S129, p-Akt S473, p-p21 T145, p-NF-κB S529, p-STAT3 Y705, or p-JAK2 Y1007/1008 polypeptide in a sample derived from a subject, wherein the sample is not exposed to the CK2 inhibitor to provide a sample profile; and (b) comparing the sample profile with a reference profile; wherein the reference profile is indicative of responsiveness to the CK2 inhibitor and/or non-responsiveness to the CK2 inhibitor.

In yet another aspect, the invention provides a method for predicting responders from non-responders for treatment of a CK2-mediated disease, such as a proliferative disorder and/or an inflammatory disorder, with a CK2 inhibitor, comprising: (a) determining the level of mRNA expression and/or polypeptide level of one or more biomarkers selected from CK2α, CK2α′, and IL-6, IL-8, VEGF, HIF-1α and/or the level of p-Akt S129, p-Akt S473, p-p21 T145, p-NF-κB S529, p-STAT3 Y705, or p-JAK2 Y1007/1008 polypeptide in a sample derived from a subject, wherein the sample is not exposed to the CK2 inhibitor to provide a sample profile; and (b) comparing the sample profile with a reference profile; wherein the reference profile is indicative of responsiveness to the CK2 inhibitor and/or non-responsiveness to the CK2 inhibitor.

In some such embodiments, step (a) comprises determining the level of IL-6 mRNA expression and/or IL-6 polypeptide in the sample derived from the subject. In some embodiments, step (a) comprises determining the level of IL-8 mRNA expression and/or IL-8 polypeptide in the sample derived from the subject. In other embodiments, step (a) comprises determining the level of CK2α and/or CK2α′ mRNA expression and/or polypeptide in the sample derived from the subject. In further embodiments, step (a) comprises determining the level of p-Akt S129 polypeptide in the sample derived from the subject. In some such embodiments, the normalized level of p-Akt S129 polypeptide is used, by determining the level of p-Akt S129 relative to total Akt polypeptide. In other embodiments, step (a) comprises determining the level of p-Akt S473 polypeptide in the sample derived from the subject. In some such embodiments, the normalized level of p-Akt S473 polypeptide is used, by determining the level of p-Akt S473 relative to total Akt polypeptide. In further embodiments, step (a) comprises determining the level of p-p21 T145 polypeptide in the sample derived from the subject. In some such embodiments, the normalized level of p-p21 T145 polypeptide is used, by determining the level of p-p21 T145 relative to total p21 polypeptide. In further embodiments, step (a) comprises determining the level of p-NF-κB S529 polypeptide in the sample derived from the subject. In some such embodiments, the normalized level of p-NF-κB S529 polypeptide is used, by determining the level of p-NF-κB S529 relative to total NF-κB polypeptide. In further embodiments, step (a) comprises determining the level of p-STAT3 Y705 polypeptide in the sample derived from the subject. In some such embodiments, the normalized level of p-STAT3 Y705 polypeptide is used, by determining the level of p-STAT3 Y705 relative to total STAT3 polypeptide. In further embodiments, step (a) comprises determining the level of p-JAK2 Y1007/1008 polypeptide in the sample derived from the subject. In some such embodiments, the normalized level of p-JAK2 Y1007/1008 polypeptide is used, by determining the level of p-JAK2 Y1007/1008 relative to total JAK2 polypeptide.

In some embodiments, similarity between the sample profile and the reference profile predicts whether the patient is a responder or non-responder to the drug for treating the CK2-mediated disease. In some embodiments, the reference profile indicative of responsiveness to the drug is obtained from one or more patients who are responsive to the drug. In other embodiments, the reference profile indicative of non-responsiveness to the drug is obtained from one or more patients who are non-responsive to the drug. In frequent embodiments, the drug is a CK2 inhibitor.

The methods provided herein can also be used to identify or predict subjects for whom treatment with a CK2 inhibitor is likely to be effective, and thus to select an individual subject or a group, or population of subjects who are likely to benefit from such treatment. Once identified, such subjects can then be selected for treatment and/or treated with a CK2 inhibitor. Conversely, subjects who are determined to be unlikely to benefit from treatment with a CK2 inhibitor can be identified and excluded from treatment with a CK2 inhibitor or provided an appropriate alternative treatment. In various embodiments described herein, the subject can be a human or other mammal In exemplary embodiments, the subject is a human subject.

### Comparison of Biomarkers to Reference Populations for Monitoring Responsiveness

In an additional aspect, the invention provides a method for monitoring the responsiveness of a CK2-mediated disease in a subject to treatment with a CK2 inhibitor, comprising: (a) determining the level of one or more biomarkers in a biological sample derived from the subject following treatment with a CK2 inhibitor, and (b) comparing the level of one or more biomarkers in the biological sample to the levels of one or more biomarkers obtained from a reference population of individuals suffering from said CK-2 mediated disease, wherein a decrease in the level of one or more biomarkers in the biological sample is indicative of a response to treatment of the CK2-mediated disease to treatment with a CK2 inhibitor.

To correlate a subject's biological sample to a standard reference population, it is necessary to obtain data on the clinical responses exhibited by a population of individuals who received the treatment, i.e., a clinical population, before and/or after treatment with the CK2 inhibitor. This clinical data maybe obtained by retrospective analysis of the results of a clinical trial(s). Alternatively, the clinical data may be obtained by designing and carrying out one or more new clinical trials. The analysis of clinical population data is useful to define a standard reference populations which, in turn, is useful to classify subjects for selection of therapeutic treatment, and/or to classify subjects as exhibiting a positive response to treatment with a CK2 inhibitor. In a preferred embodiment, the subjects included in the clinical population have been graded for the existence of the medical condition of interest, e.g., a CK2-mediated disease. Grading of potential subjects can include, e.g., a standard physical exam or one or more lab tests. Alternatively, grading of subjects can include use of a gene expression pattern, a protein expression pattern, or a phosphorylation pattern. For example, gene expression pattern is useful as grading criteria where there is a strong correlation between gene expression pattern and disease susceptibility or severity. Such standard reference population comprising subjects sharing gene expression pattern profile characteristic(s). For example, biomarker gene expression characteristic(s), are useful in the methods of the present invention to compare with the measured level of one or more gene expression product in a given subject. This gene expression product(s) useful in the methods of the present invention include, but are not limited to, e.g., characteristic mRNA associated with that particular genotype group or the polypeptide gene expression product of that genotype group. In one embodiment, a subject is classified or assigned to a particular genotype group or class based on similarity between the measured levels of a one or more biomarkers in the subject and the level of the one or more biomarkers observed in a standard reference population.

In an exemplary embodiment, the biomarker is selected from the mRNA expression and/or polypeptide level of CK2α, CK2α′, IL-6, IL-8, VEGF, or HIF-1α, or the level of p-Akt S129, p-Akt S473, p-p21 T145, p-NF-κB S529, p-STAT3 Y705, or p-JAK2 Y1007/1008 polypeptide. In another embodiment, combinations of two or more biomarkers are used, and selected from the mRNA expression and/or polypeptide level of CK2α, CK2α′, IL-6, IL-8, VEGF, or HIF-1α, or the level of p-Akt S129, p-Akt S473, p-p21 T145, p-NF-κB S529, p-STAT3 Y705, or p-JAK2 Y1007/1008 polypeptide.

It will be understood that in the methods described herein relating to the levels of p-Akt S129, p-Akt S473, p-p21 T145, p-NF-κB S529, p-STAT3 Y705, or p-JAK2 Y1007/1008, either the absolute level or the normalized level of the p-Akt S129, p-Akt S473, p-p21 T145, p-NF-κB S529, p-STAT3 Y705, or p-JAK2 Y1007/1008 polypeptide, respectively, may be used.

Combinations of Biomarkers for Predicting Sensitivity and/or Monitoring Responsiveness

In various embodiments described herein, the methods of the present invention can utilize one or more combinations of biomarkers identified herein for predicting the sensitivity and/or monitoring the responsiveness of a CK2-mediated disease to treatment with a CK2 inhibitor.

Thus, in one embodiment, the present invention provides a combination of tests useful for predicting or determining the treatment efficacy of a CK2 inhibitor comprising a first test for detecting the level of a first biomarker of a biological sample from a subject and a second test for detecting the level of a second biomarker of said biological sample, wherein the first marker is different from the second marker. In one embodiment, the first biomarker is selected from the mRNA expression and/or polypeptide level of CK2ζ, CK2α′, IL-6, IL-8, VEGF, or HIF-1α, or the level of p-Akt S129, p-Akt S473, p-p21 T145, p-NF-κB S529, p-STAT3 Y705, or p-JAK2 Y1007/1008 polypeptide. In a further embodiment, the second biomarker is selected from the mRNA expression and/or polypeptide level of CK2α, CK2α′, IL-6, IL-8, VEGF, or HIF-1α, or the level of p-Akt S129, p-Akt S473, p-p21 T145, p-NF-κB S529, p-STAT3 Y705, or p-JAK2 Y1007/1008 polypeptide.

In another embodiment, the present invention provides a combination of biomarkers (i.e. a biomarker panel) useful for predicting or determining the treatment efficacy of a CK2 inhibitor. In one embodiment, the biomarker panel includes one or more biomarkers selected from the mRNA expression and/or polypeptide level of CK2α, CK2α′, IL-6, IL-8, VEGF, or HIF-1α, or the level of p-Akt S129, p-Akt S473, p-p21 T145, p-NF-κB S529, p-STAT3 Y705, or p-JAK2 Y1007/1008 polypeptide. In another embodiment, the biomarker panel includes two, three, four, five, six, seven, eight, nine, ten, or more biomarkers selected from the mRNA expression and/or polypeptide level of CK2α, CK2α′, IL-6, IL-8, VEGF, or HIF-1α, or the level of p-Akt S129, p-Akt S473, p-p21 T145, p-NF-κB S529, p-STAT3 Y705, or p-JAK2 Y1007/1008 polypeptide. In an exemplary embodiment, the biomarker panel includes all of the biomarkers selected from the mRNA expression and/or polypeptide level of CK2α, CK2α′, IL-6, IL-8, VEGF, or HIF-1α, or the level of p-Akt S129, p-Akt S473, p-p21 T145, p-NF-κB S529, p-STAT3 Y705, or p-JAK2 Y1007/1008 polypeptide.

In another embodiment, the present invention provides a method of providing useful information for predicting or determining the treatment efficacy of a CK2 inhibitor comprising determining the level of one or more biomarkers from a biological sample of a subject and providing the level of one or more biomarkers to an entity that provides a prediction or determination of the therapeutic efficacy based on an increase or decrease in the level of one or more biomarkers in a subject treated with a CK2 inhibitor. In one embodiment, the biomarker is selected from the mRNA expression and/or polypeptide level of CK2α, CK2α′, IL-6, IL-8, VEGF, or HIF-1α, or the level of p-Akt S129, p-Akt S473, p-p21 T145, p-NF-κB S529, p-STAT3 Y705, or p-JAK2 Y1007/1008 polypeptide.

### Methods of Screening Subjects to Predict Responsiveness

The present invention thus provides a method of screening subjects suffering from a proliferative disorder in order to predict their responsiveness to treatment with a CK2 inhibitor, comprising determining the level of mRNA expression and/or polypeptide levels of the CK2 catalytic subunits (CK2α/CK2α′), IL-6, IL-8, VEGF, HIF-1α and/or the phosphorylation status of p-Akt S129, p-Akt S473, p-p21 T145, p-NF-κB S529, p-STAT3 Y705, and p-JAK2-Y1007/1008 by a method as defined above.

In a further aspect, the present invention provides a method of treating a proliferative and/or inflammatory disorder in a subject in need thereof, comprising determining the level of expression of the CK2 catalytic subunits (CK2α/CK2α′), IL-6, IL-8, VEGF, HIF-1α and/or the phosphorylation status of Akt, preferably Akt S129 or Akt S473, or p21, preferably T145, or NF-κB, preferably S529, STAT3, preferably Y705, or JAK2, preferably Y1007 or Y1008, in a sample derived from the subject, by the methods described herein, and treating the subject with a CK2 inhibitor if the level of expression of CK2 catalytic subunits, IL-6, IL-8, VEGF, HIF-1α and/or phosphorylated Akt, p21, NF-κB, STAT3, or JAK2 is elevated.

The level determined for a particular biomarker or biomarkers in a biological sample, such as a cell or tissue, a system or subject may be compared with an appropriate control sample. For example, a control sample may comprise a biological sample derived from a subject not suffering from the disease, or a sample of normal tissue (i.e., non-tumorous tissue) from the same subject.

Elevated levels of mRNA expression and/or polypeptide levels for CK2α, CK2α′ and/or IL-6, IL-8, VEGF, HIF-1α and/or an elevated level of phosphorylated Akt, p21, NF-κB, STAT3, or JAK2, either alone or relative to total Akt, p21, NF-κB, STAT3, or JAK2, respectively have been found to be predictive of a beneficial therapeutic effect of a CK2 inhibitor. The elevated level at which therapeutic use of a CK2 inhibitor is indicated may be determined by a skilled person. In certain embodiments, treatment with CK2 inhibitor may be indicated where the elevated level in the sample is detectably above the control level, or where the level is at least 50%, 75%, 100%, 300%, 500% or 1000% higher than control.

In some embodiments, the appropriate control will be a control sample obtained from a normal subject or a group of subjects who are not afflicted with the proliferative disorder and/or the inflammatory disorder. Sometimes, the appropriate control may be a control sample from a normal cell or tissue of the subject afflicted by the proliferative disorder and/or the inflammatory disorder. For example, in a subject afflicted by cancer, the test biological sample may be derived from a tumor in the tissue affected by cancer, and the control sample may be obtained from a tissue that is not affected by the cancer. Control samples can be assessed for the level of mRNA expression and/or the polypeptide level of the biomarker(s) of interest, or the phosphorylation status of the biomarker, and compared to the corresponding levels for the biomarker(s) of interest in the test biological sample.

When the methods relate to the prediction of sensitivity or responsiveness to a CK2 inhibitor, the subject is typically a subject who has been identified or diagnosed as having a CK2-mediated disease, such as a proliferative disorder and/or an inflammatory disorder, and who has not undergone treatment with a CK2 inhibitor. Thus, the methods can be used to predict which subjects are likely to be responsive to treatment with a CK2 inhibitor prior to initiating treatment. In other embodiments, the subject has been administered a CK2 inhibitor, and the subject is being assessed to monitor the effectiveness of treatment.

### Methods of Selecting Dosages Using the Identified Biomarkers

The methods of the present invention may also be used to select an appropriate dose of a CK2 inhibitor to individually optimize therapy for each subject. Factors to be considered in selecting the appropriate dose include the particular subject and condition being treated, the clinical condition of the individual patient, the site of delivery of the active compound, the particular type of the active compound, the method of administration, the scheduling of administration, the severity of the condition and other factors known to medical practitioners. The therapeutically effective amount of an active compound to be administered will be governed by such considerations, and is the minimum amount necessary to prevent, ameliorate, or treat the disease. Such amount is preferably below the amount that is toxic to the host or which renders the host significantly more susceptible to infections.

### The Biological Sample

As described herein, the methods relate to the determination of biomarker levels in a system. The system may be in vitro or in vivo. Thus, the methods may be performed in vivo or in vitro, e.g., on a biological sample derived from a subject, including but not limited to a mammalian subject, such as a human subject. In one embodiment, the biological sample is a biological material derived from the subject such as e.g., a cell (e.g. a circulating tumor cell), cell line, tissue (e.g. a biopsy tissue), tissue culture, cell or tissue lysate, tumor, or a biological fluid or a fraction thereof, such as plasma, serum, blood, urine, saliva, or peripheral blood mononuclear cells (PBMCs), for example lymphocyte or monocyte PBMCs. In some embodiments, the PBMCs are separated into phenotypes, such as CD19 positive (CD19+) or CD45 positive (CD45+) PBMCs. PBMCs can be isolated or extracted from whole blood using methods known to those of skill in the art, for example, through the use of ficoll or by hypotonic lysis.

### Biomarker Measurement

Expression levels and/or phosphorylation for the biomarkers described herein are assayed in the biological sample by any technical means on the basis of RNA expression using for example the technique of RT-PCR and DNA microarray, or on the basis of protein expression (i.e. to measure polypeptide levels) using for example the technique of Western blotting, immunohistochemistry or ELISA, including immunoassays, immunoprecipitation and electrophoresis assays.

Antibodies specific for the CK2 catalytic subunits (CK2α/CK2α′), IL-6, IL-8, VEGF, HIF-1α, Akt, p-Akt S129, p-Akt S473, p21, p-p21 T145, NF-κB, p-NF-κB S529, STAT3, p-STAT3 Y705, JAK2, and p-JAK2-Y1007/1008 may be used in a standard immunoassay format to measure expression levels. For instance, ELISA (enzyme linked immunosorbent assay) type assays, immunoprecipitation type assays, conventional Western blotting assays, immunofluorescence assays and immunohistochemistry assays using monoclonal or polyclonal antibodies can also be utilized to determine levels of the CK2 catalytic subunits (CK2α/CK2α′), IL-6, IL-8, VEGF, HIF-1α, Akt, p-Akt S129, p-Akt S473, p21, p-p21 T145, NF-κB, p-NF-κB S529, STAT3, p-STAT3 Y705, JAK2, and p-JAK2-Y1007/1008 as biomarker proteins. Polyclonal and monoclonal antibodies specific to these biomarkers may be produced in accordance with known methods.

Biomarker levels can also be measured using two-dimensional (2-D) gel electrophoresis, and then analyzed, e.g., by immunoblot analysis using antibodies, using methods known in the art.

### CK-2 Mediated Diseases

In frequent embodiments of the present invention, the CK2-mediated disease is a proliferative disorder and/or an inflammatory disorder. In some embodiments, the proliferative disorder comprises cancer. The cancer can be cancer of the breast, prostate, colon, rectum, pancreas, liver, brain, head and neck, lung (SCLC or NSCLC), or skin (e.g., melanoma). In specific embodiments, the cancer is prostate cancer or breast cancer. In certain embodiments, the cancer is inflammatory breast cancer. In other embodiments, the disorder is acute or chronic myelogenous leukemia, acute lymphoblastic, chronic lymphocytic leukemia, Bcr/Abl-positive leukemia, lymphoma, or multiple myeloma. In other embodiments, the disorder is a solid tumor, including an advanced solid tumor. In other embodiments, the disorder is Castleman's disease.

In other embodiments, the disorder described herein is an inflammatory disorder. Sometimes, the inflammatory disorder is glomerulonephritis, multiple sclerosis, systemic lupus erythematosus, rheumatoid arthritis, or juvenile arthritis. In some embodiments, the compounds are used to alleviate inflammatory pain, since murine models demonstrate that CK2 modulates nociceptive signal transmission, and reduces pain response in mice when infused into the spinal cord.

In alternative embodiments, the CK2-mediated disorder is selected from the group consisting of a neurodegenerative disorder, pain, a disorder of the vascular system, a pathophysiological disorder of skeletal muscle or bone tissue, protozoan parasitosis, or a viral disease.

In certain embodiments, the CK2-mediated disorder is a neurodegenerative disorder. In some such embodiments, the neurodegenerative disorder is Alzheimer's disease, Parkinson's disease, memory impairment, brain ischemia, Guam-Parkinson dementia, chromosome 18 deletion syndrome, progressive supranuclear palsy, Kuf's disease, or Pick's disease.

In further embodiments, the CK2-mediated disorder is a disorder of the vascular system. In some such embodiments, the disorder of the vascular system is atherosclerosis, laminar shear stress or hypoxia.

In other embodiments, the CK2-mediated disorder is a pathophysiological disorder of skeletal muscle or bone tissue. These conditions include atherosclerosis, laminar shear stress, and hypoxia and associated conditions. In some such embodiments, the disorder is cardiomyocyte hypertrophy, impaired insulin signaling or bone tissue mineralization.

In still other embodiments, the disorder is a protozoan parasitosis. Infections by protozoans have been shown to lead to almost immediate increases in IL-8 levels in the infected host.

In addition to the involvement of CK2 inhibitors in the life cycle of such pathogens, which is discussed above, the suppression of IL-8 expression may be helpful in ameliorating localized injury associated with parasitic pathogens. The compounds of the invention are thus useful for treatment of parasitosis due to Theileria parva; Toxoplasma gondii, Trypanosoma cruzi (Chagas disease), Leishmania donovani, Herpetomonas muscarum muscarum, Plasmodium falciparum, Traypanosoma brucei, and Schistosoma mansoni, among others.

In further embodiments, the disorder is a viral disease. In some such embodiments, the viral disease is human immunodeficiency virus type 1 (HIV-1), human papilloma virus, Epstein-Barr virus or herpes simplex virus. In other embodiments, the viral disorder is human papilloma virus, human cytomegalovirus, hepatitis C or B, Borna disease virus, adenovirus, coxsackie virus, coronavirus, or varicella zoster virus.

### CK2 Inhibitors

CK2 is a protein with a unique active site that can be inhibited by a variety of known therapeutics, including staurosporine, a natural product originally isolated in 1977 from Streptomyces staurosporeus (Omura et al., 1977, J. Antibiot. 30: 275-82), which inhibits protein kinases through the prevention of ATP binding to the kinase. In addition to staurosporine, many ATP-competitive inhibitors of CK2 have been reported in the literature, including 5,6-dichloro-1-β-D-ribofuranosylbenzimidazole (DRB), 6-methyl-1,3,8-trihydroxyanthraquinone (emodin), 2-dimethylamino-4,5,6,7-tetrabromo-1H-benzimidazole (DMAT), 4,5,6,7-tetrabromobenzotriazole (TBB), resorufin, 4,4′,5,5′,6,6′-Hexahydroxydiphenic acid 2,6,2′,6′-dilactone (ellagic acid), [5-oxo-5,6-dihydroindolo-(1,2-a)quinazolin-7-yl]acetic acid (IQA), and 2-(3,4-dihydroxyphenyl)-3,5,7-trihydroxy-4H-chromen-4-one (quercetin). See, e.g., Zhu et al., 2009, Mol. Cell. Biochem. 333: 159-67; Lopez-Ramos et al., 2010, Faseb J. 24: 3171-85; and Cozza et al., 2010, Med. Res. Rev. 30: 419-62.

CK2 inhibitors exert biological activities that include, but are not limited to, inhibiting cell proliferation and modulating protein kinase activity. CK2 inhibitors can modulate protein kinase CK2 activity, and without being bound by theory, it is believed their inhibition of CK2 provides the ability to treat various disorders described herein, which are associated with aberrant, excessive, or undesired levels of CK2 activity. Such compounds therefore can be utilized in multiple applications by a person of ordinary skill in the art. For example, CK2 inhibitors may find uses that include, but are not limited to, (i) modulation of protein kinase activity (e.g., CK2 activity), (ii) modulation of cell proliferation, (iii) modulation of apoptosis, (iv) treatment of cell proliferation related disorders, such as leukemia, lymphoma, multiple myeloma, and solid tumors (e.g., tumors of the breast or prostate), and (v) treatment of neurodegenerative disorders, inflammatory disorders, disorders of the vascular system, disorders of skeletal muscle or bone tissue, protozoan parasitosis, viral diseases, and pain.

A CK2 inhibitor can be formulated as a pharmaceutical composition. Such a pharmaceutical composition can then be administered by any suitable route of administration, for example, orally, parenterally, by inhalation spray, rectally, or topically in dosage unit formulations containing conventional nontoxic pharmaceutically acceptable carriers, adjuvants, and vehicles as desired. Formulation of drugs is discussed in, for example, Hoover, John E., REMINGTON′S PHARMACEUTICAL SCIENCES, Mack Publishing Co., Easton, Pa.; 1975. Other examples of drug formulations can be found in Liberman, H. A. and Lachman, L., Eds., PHARMACEUTICAL DOSAGE FORMS, Marcel Decker, New York, N.Y., 1980. Pharmaceutical compositions suitable for use in the present invention include compositions wherein the active ingredients are contained in an effective amount to achieve the intended purpose. Determination of the effective amounts and appropriate dosing regimens is within the capability of those skilled in the art.

A CK2 inhibitor may be in a therapeutically effective amount in a pharmaceutical composition, formulation or medicament, which is an amount that can lead to a desired biological effect, leading to ameliorating, alleviating, lessening, or removing symptoms of a disease or condition. The terms also can refer to reducing or stopping a cell proliferation rate (e.g., slowing or halting tumor growth) or reducing the number of proliferating cancer cells (e.g., removing part or all of a tumor).

CK2 inhibitors as described herein include, but are not limited to, the compounds of any of the formulae described in International Patent Application Nos. PCT/US2007/077464, PCT/US2008/074820, and PCT/US2009/035609, and U.S. Provisional Application Ser. Nos. 61/170,468 (filed 17 Apr. 2009), 61/242,227 (filed 14 Sep. 2009), 61,180,090 (filed 20 May 2009), 61/218,318 (filed 18 Jun. 2009), 61/179,996 (filed 20 May 2009), 61/218,214 (filed 14 Jun. 2009), 61/41,806 (11 Sep. 2009), 61/180,099 (filed 20 May 2009), 61/218,347 (filed 18 Jun. 2009), 61/237,227 (filed 26 Aug. 2009), 61/243,107 (filed 16 Sep. 2009) and 61/243,104 (filed 16 Sep. 2009), the contents of each of which are incorporated herein by reference in their entirety. CK2 inhibitors can be synthesized by methods known in the art, including methods disclosed in International Patent Application Nos. PCT/US2007/077464, PCT/US2008/074820, and PCT/US2009/035609.

In one embodiment of the present invention, the CK2 inhibitor is a compound having structural Formula (A):

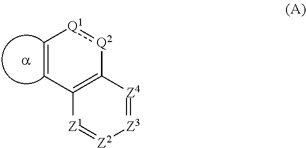

or a pharmaceutically acceptable salt, solvate, and/or prodrug thereof;

wherein the group labeled a represents a 5- or 6-membered aromatic or heteroaromatic ring fused onto the ring containing Q¹, wherein α is a 6-membered aryl ring optionally containing one or more nitrogen atoms as ring members, or a 5-membered aryl ring selected from thiophene and thiazole;

Q¹ is C═X, Q² is NR⁵, and the bond between Q¹ and Q² is a single bond; or Q¹ is C—X—R⁵, Q² is N, and the bond between Q¹ and Q² is a double bond; and

wherein X represents O, S or NR⁴;

each Z¹, Z², Z³, and Z⁴ is N or CR³ and one or more of Z¹, Z², Z³, and Z⁴ is CR³;

each R³ is independently H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,

or each R³ is halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,
wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
and wherein two R on the same atom or on adjacent atoms can be linked to form a 3-8 membered ring, optionally containing one or more N, O or S;
   and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
      wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
   and wherein two R′ can be linked to form a 3-7 membered ring optionally containing up to three heteroatoms selected from N, O and S,

R⁴ is H or optionally substituted member selected from the group consisting of C₁-C₆ alkyl, C2-C6 heteroalkyl, and C1-C6 acyl;

each R⁵ is independently H or an optionally substituted member selected from the group consisting of C₁₋₁₀ alkyl, C₂₋₁₀ alkenyl, C₂₋₁₀ heteroalkyl, C₃₋₈ carbocyclic ring, and C₃₋₈ heterocyclic ring optionally fused to an additional optionally substituted carbocyclic or heterocyclic; or R⁵ is a C₁₋₁₀ alkyl, C₂₋₁₀ alkenyl, or C₂₋₁₀ heteroalkyl substituted with an optionally substituted C₃₋₈ carbocyclic ring or C₃₋₈ heterocyclic ring; and
in each —NR⁴R⁵, R⁴ and R⁵ together with N may form an optionally substituted 3-8 membered ring, which may optionally contain an additional heteroatom selected from N, O and S as a ring member;

provided that when Q¹ in Formula (A) is C—NHΦ, where Φ is optionally substituted phenyl:
if the ring labeled a is a six-membered ring containing at least one N as a ring member, at least one R³ present must be a polar substituent, or if each R³ is H, then Φ must be substituted; and

if the ring labeled a is phenyl, and three of Z¹-Z⁴ represent CH, then Z² cannot be C—OR″, and Z³ cannot be NH₂, NO₂, NHC(═O)R″ or NHC(═O)-OR″, where R″ is C1-C4 alkyl

In one embodiment of Formula (A), the compound is represented by structural Formula I, II, III or IV:

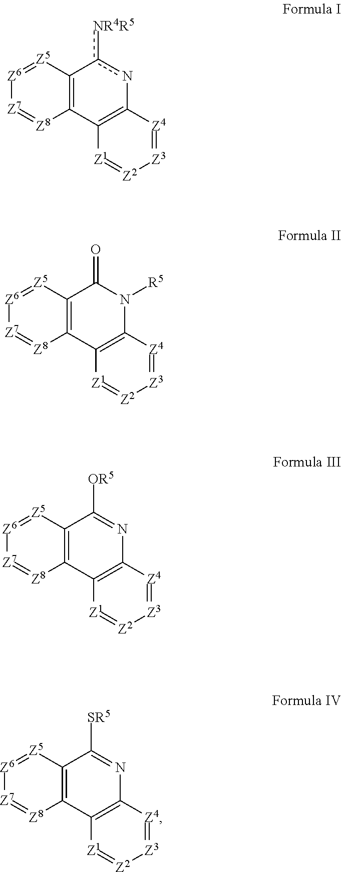

or a pharmaceutically acceptable salt, solvate, and/or prodrug thereof;

wherein:

each Z¹, Z², Z³, and Z⁴ is N or CR³;

each of Z⁵, Z⁶, Z⁷ and Z⁸ is N or CR⁶;

none, one or two of Z¹-Z⁴ are N and none, one or two of Z⁵-Z⁸ are N;
each R³ and each R⁶ is independently H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,
or each R³ and each R⁶ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, polar substituent, carboxy bioisostere, COOH or NO₂,
   wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,
      and wherein two R on the same atom or on adjacent atoms can be linked to form a 3-8 membered ring, optionally containing one or more N, O or S;
      and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂,
         wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;
      and wherein two R′ can be linked to form a 3-7 membered ring optionally containing up to three heteroatoms selected from N, O and S;

R⁴ is H or an optionally substituted member selected from the group consisting of C1-C6 alkyl, C2-C6 heteroalkyl, and C1-C6 acyl;

each R⁵ is independently H or an optionally substituted member selected from the group consisting of C₁₋₁₀ alkyl, C₂₋₁₀ alkenyl, C₂₋₁₀ heteroalkyl, C₃₋₈ carbocyclic ring, and C₃₋₈ heterocyclic ring optionally fused to an additional optionally substituted carbocyclic or heterocyclic ring; or R⁵ is a C₁₋₁₀ alkyl, C₂₋₁₀ alkenyl, or C₂₋₁₀ heteroalkyl substituted with an optionally substituted C₃₋₈ carbocyclic ring or C₃₋₈ heterocyclic ring; and
in each —NR⁴R⁵, R⁴ and R⁵ together with N may form an optionally substituted 3-8 membered ring, which may optionally contain an additional heteroatom selected from N, O and S as a ring member;
provided that when —NR⁴R⁵ in Formula (I) is —NHΦ, where Φ is optionally substituted phenyl:
   if all of Z⁵-Z⁸ are CH or one of Z⁵-Z⁸ is N, at least one of Z¹-Z⁴ is CR³ and at least one R³ must be a non-hydrogen substituent; or
   if each R³ is H, then Φ must be substituted; or

if all of Z⁵-Z⁸ are CH or one of Z⁵-Z⁸ is N, then Z² is not C—OR″, and Z³ is not NH₂, NO₂, NHC(═O)R″ or NHC(═O)—OR″, where R″ is C1-C4 alkyl.

In one embodiment of Formula I, the compound is represented by structural Formulae Ia, Ib, Ic or Id:

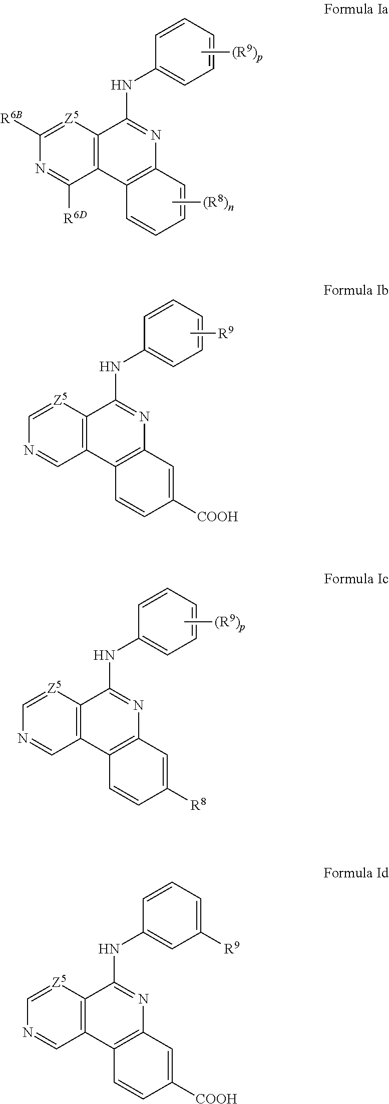

or a pharmaceutically acceptable salt, solvate, and/or prodrug thereof; wherein:

Z⁵ is N or CR^{6A};

each R^{6A}, R^{6B}, R⁶ and R⁸ independently is H or an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group,

or each R^{6A}, R^{6B}, R⁶ and R⁸ independently is halo, CF₃, CFN, OR, NR₂, NROR, NRNR₂, SR, SOR, SO₂R, SO₂NR₂, NRSO₂R, NRCONR₂, NRCOOR, NRCOR, CN, COOR, carboxy bioisostere, CONR₂, OOCR, COR, or NO₂,

R⁹ is independently an optionally substituted C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C12 heteroaryl, C7-C12 arylalkyl, or C6-C12 heteroarylalkyl group, or

R⁹ is independently halo, OR, NR₂, NROR, NRNR₂, SR, SOR, SO₂R, SO₂NR₂, NRSO₂R, NRCONR₂, NRCOOR, NRCOR, CN, COOR, CONR₂, OOCR, COR, or NO₂,

wherein each R is independently H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl,

and wherein two R on the same atom or on adjacent atoms can be linked to form a 3-8 membered ring, optionally containing one or more N, O or S;

and each R group, and each ring formed by linking two R groups together, is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O;

and wherein two R′ can be linked to form a 3-7 membered ring optionally containing up to three heteroatoms selected from N, O and S;

n is 0 to 4; and

p is 0 to 4.

In certain embodiments of Formula I, the compound is selected from the group consisting of:

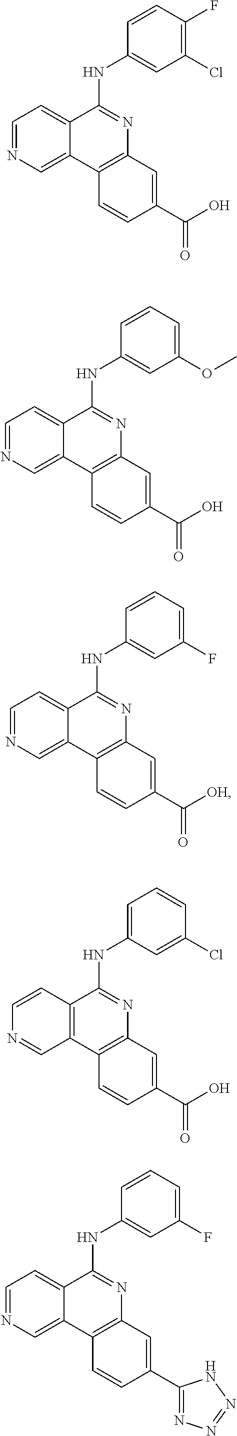

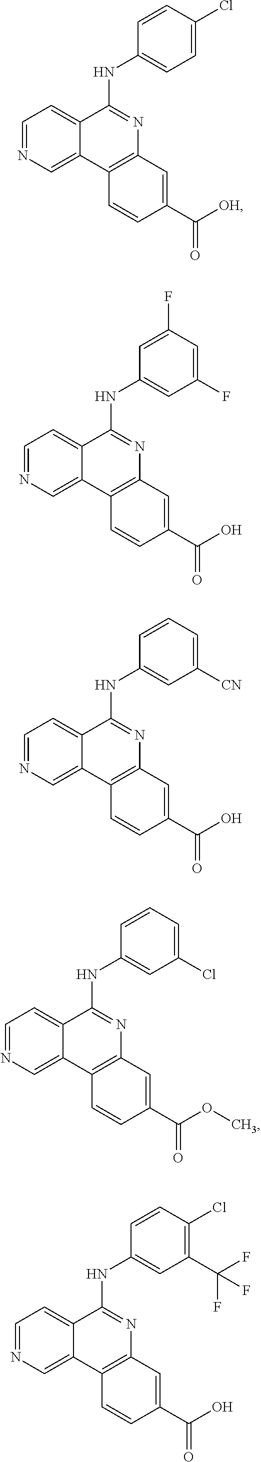

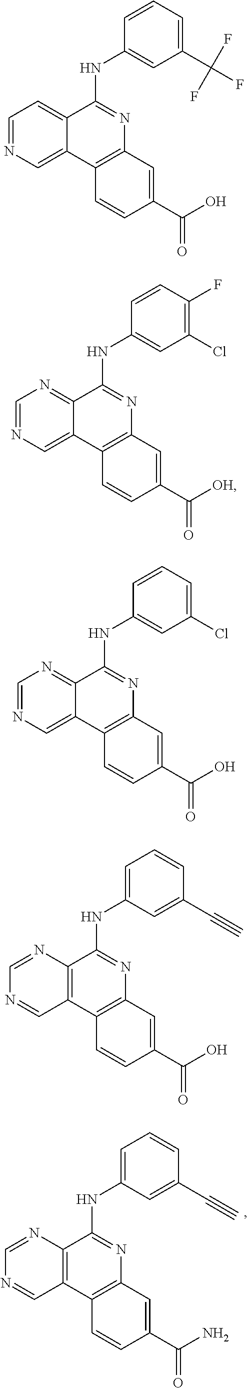

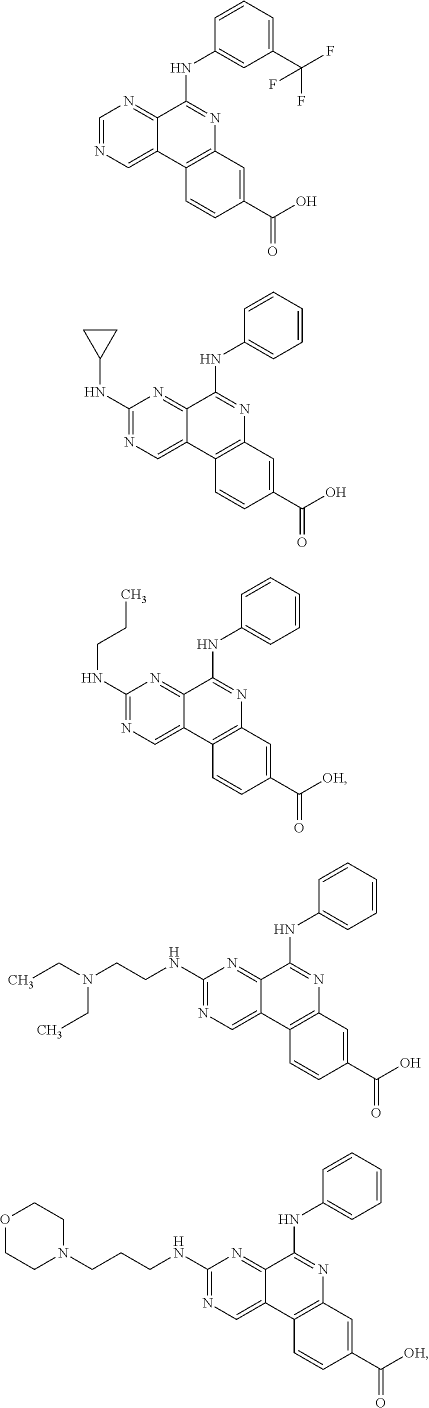

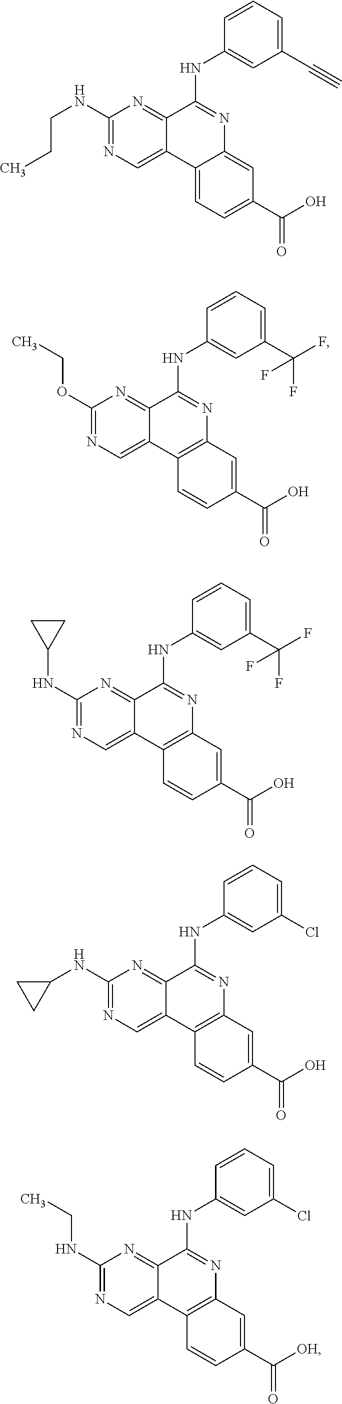

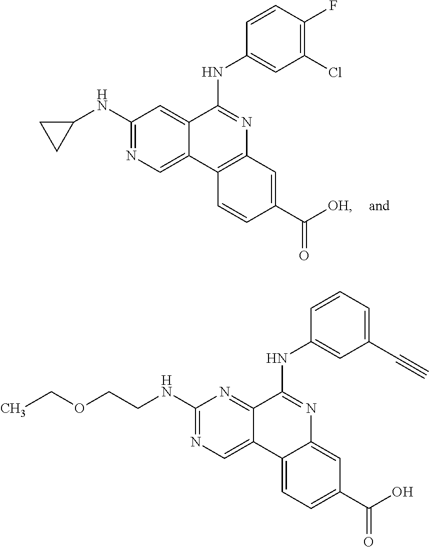

In specific embodiments of the methods described herein, the CK2 inhibitor is Compound K (CX-4945):

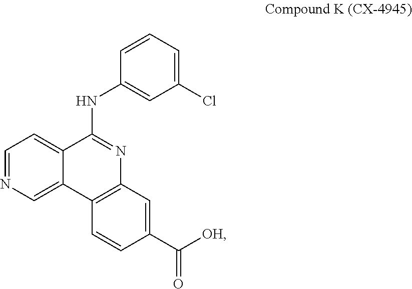

or a pharmaceutically acceptable salt or ester thereof.

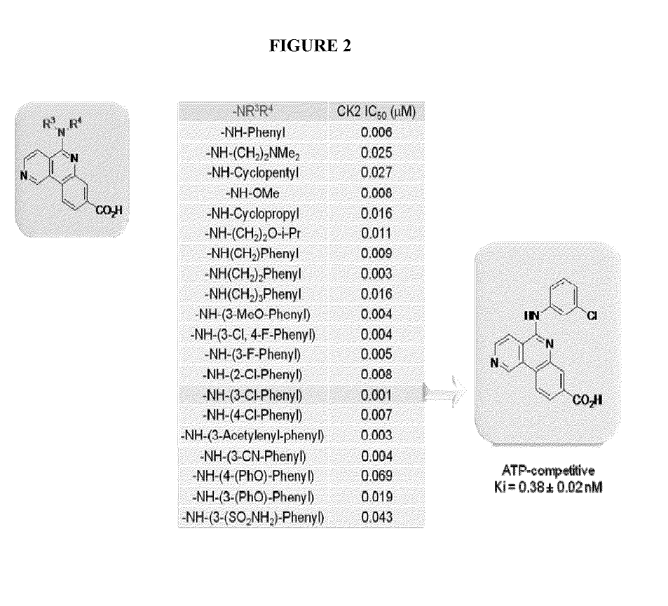

As used herein, the term “Compound K” is used interchangeably with CX-4945 and refers to a first-in-class potent, selective and orally available ATP-competitive inhibitor of CK2 with favorable drug properties. CX-4945 is currently being investigated for the treatment of several different cancer types, including advanced solid tumors, Castleman's disease, and multiple myeloma. See, e.g., “CX-4945, an Orally Bioavailable Selective Inhibitor of Protein Kinase CK2, Inhibits Survival and Angiogenic Signaling and Exhibits Antitumor Efficacy”, Siddiqui-Jain, A. et al., Cancer Research, submitted for publication; and “Discovery and Structure Activity Relationship of CX-4945, a First-In-Class Potent, Selective and Orally Available Inhibitor of Protein Kinase CK2 for the Treatment of Cancer”, Pierre, F. et al., J. Med. Chem., to be submitted. CX-4945 is an extremely potent CK2 inhibitor, with a CK2 IC₅₀ of 0.001 μM. See FIG. 2, which shows the CK2 inhibitory activity of CX-4945 in comparison to various CX-4945 analogs. As shown in Table 2, CX-4945 has high specificity for the CK2α and CK2α′ subunits.

TABLE 2
CX-4945 is a Highly Selective CK2 Inhibitor.

| Kinase | IC₅₀ (nM) |
|---|---|
| CK2α | 1 |
| CK2α′ | 1 |
| DAPK3 | 17 |
| FLT3 | 35 |
| TBK1 | 35 |
| CLK3 | 41 |
| HIPK3 | 45 |
| PIM1 | 46 |
| Cdk1/Cyclin B | 56 |
| DYRK2 | 91 |
| AKT1 | >500 |
| AKT2 | >500 |
| AKT3 | >500 |
| mTOR | >500 |
| PDK1 | >500 |
| p70S6K | >500 |
| PI3K (p110β/p85α) | >500 |
| PI3K (p120γ) | >500 |
| PI3K (p110δ/p85α) | >500 |

An ongoing Phase I clinical study of CX-4945 in patients whose tumors express CK2 is described in Example 1. CX-4945 has been seen to inhibit cell proliferation in various cancer cell lines and is efficacious in multiple xenograft models of cancer. Furthermore, CX-4945 is orally available across species (% F 20-48), has no significant in vitro inhibition of 5 CYP isoforms and the hERG channel, and is non-mutagenic.

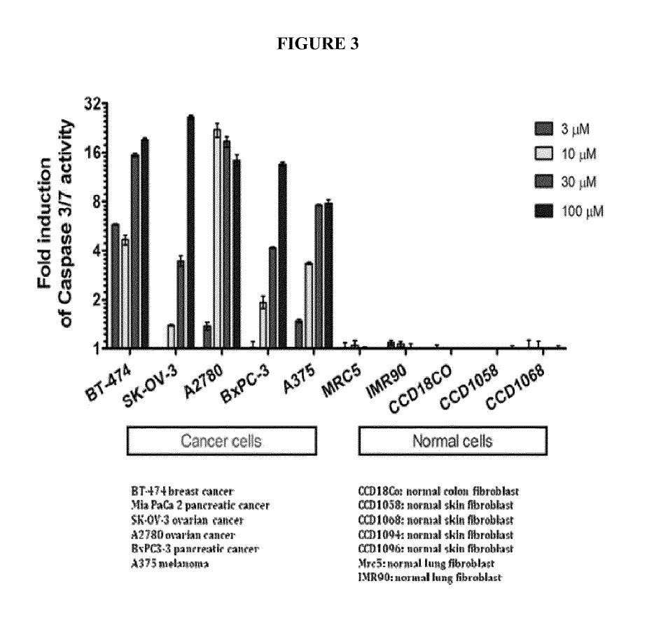

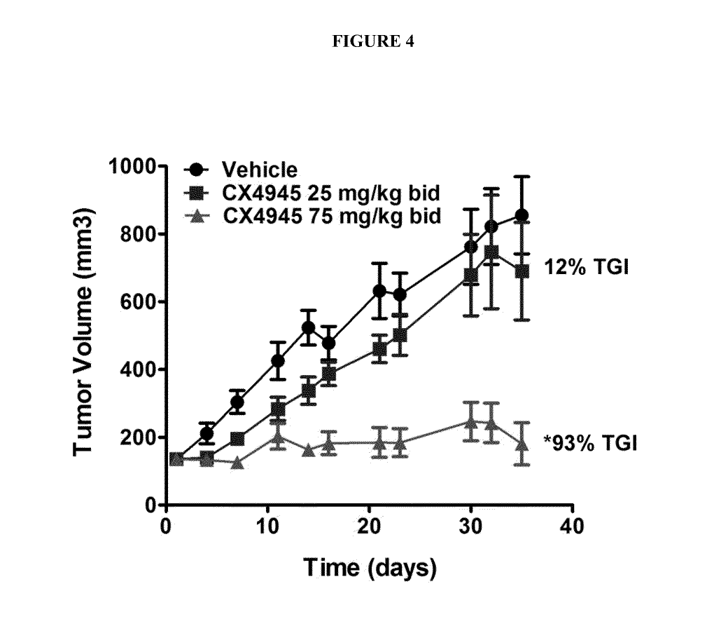

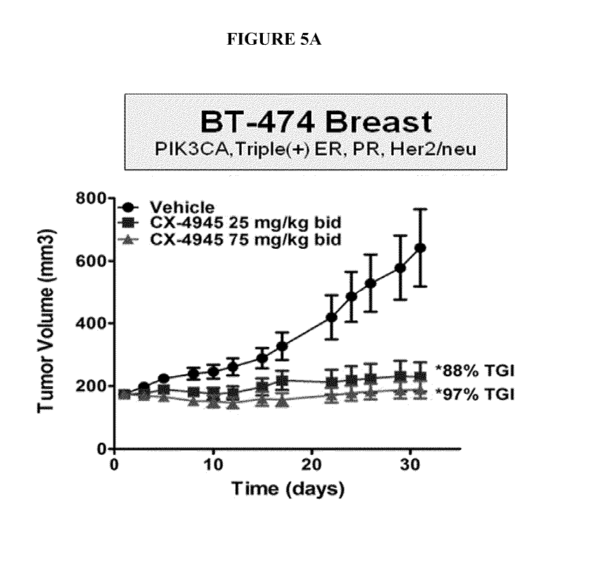

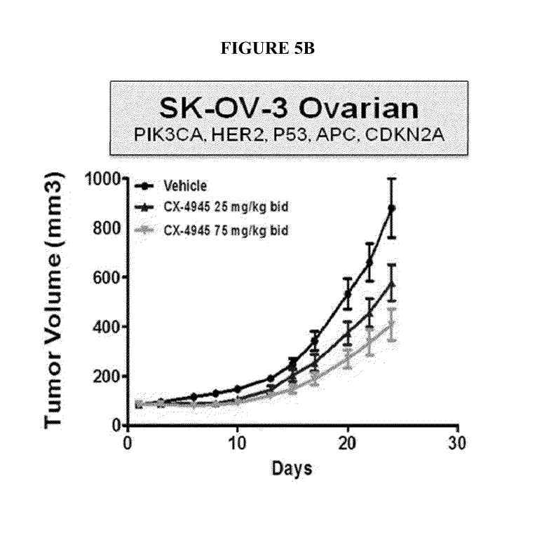

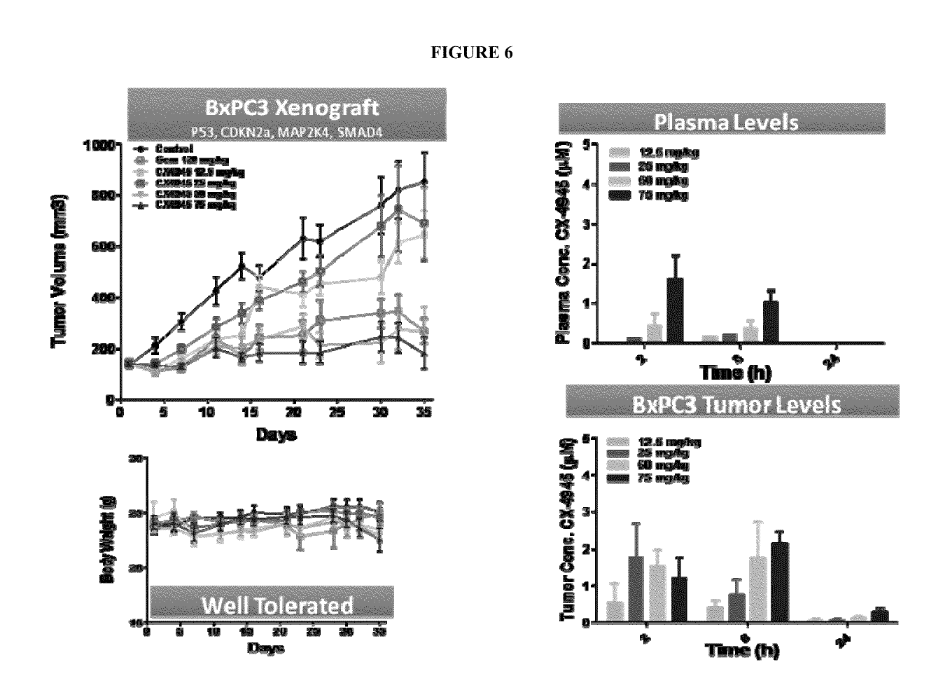

As shown in FIG. 3, CX-4945 shows differential sensitivity between cancerous and normal cells. Notably, CX-4945 induces significant levels of apoptosis in cancer cells, while normal cells remain unaffected. In vivo, CX-4945 inhibit tumor growth and pharmacodynamic markers in multiple models, including models of breast and ovarian cancer. See FIGS. 4, 5A (Breast Cancer), and 5B (Ovarian Cancer). In addition, total plasma exposure to CX-4945 correlates with reductions in tumor volume in BxPC-3 (pancreatic cancer) xenografts. See FIG. 6.

In other specific embodiments, the CK2 inhibitor is a compound (Compound 1 or Compound 2) having the formula:

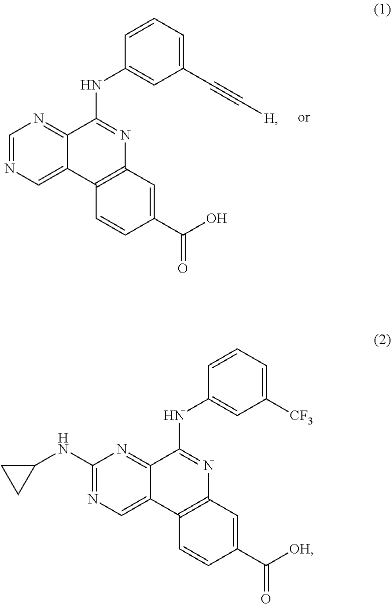

or a pharmaceutically acceptable salt or ester thereof.

Compound 1 exhibited an IC₅₀ of 6 nM for inhibition of CK2; compound 2 exhibited an IC₅₀ of about 9 nM (as compared to CX-4945, which exhibited an IC₅₀ of 1 nM for inhibition of CK2, See FIG. 1 and Table 2).

In other specific embodiments, the CK2 inhibitor is selected from DRB, emodin, DMAT, TBB, resorufin, ellagic acid, IQA, and quercetin.

The compounds of the invention as described above can be synthesized using methods, techniques, and materials known to those of skill in the art, such as described, for example, in March, ADVANCED ORGANIC CHEMISTRY 4.sup.th Ed., (Wiley 1992); Carey and Sundberg, ADVANCED ORGANIC CHEMISTY 3.sup.rd Ed., Vols. A and B (Plenum 1992), and Green and Wuts, PROTECTIVE GROUPS IN ORGANIC SYNTHESIS 2.sup.nd Ed. (Wiley 1991). Starting materials useful for preparing compounds of the invention and intermediates thereof are commercially available from sources, such as Aldrich Chemical Co. (Milwaukee, Wis.), Sigma Chemical Co. (St. Louis, Mo.), Maybridge (Cornwall, England), Asinex (Winston-Salem, N.C.), ChemBridge (San Diego, Calif.), ChemDiv (San Diego, Calif.), SPECS (Delft, The Netherlands), Timtec (Newark, Del.), or alternatively can be prepared by well-known synthetic methods (see, e.g., Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-21, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” 3d Edition, John Wiley & Sons, 1995). Other methods for synthesis of the present compounds and/or starting materials thereof are either described in the art or will be readily apparent to the skilled artisan. Alternatives to the reagents and/or protecting groups may be found in the references provided above and in other compendiums well known to the skilled artisan.

Preparation of the present compounds may include one or more steps of protection and deprotection (e.g., the formation and removal of acetal groups). Guidance for selecting suitable protecting groups can be found, for example, in Greene & Wuts, “Protective Groups in Organic Synthesis,” Wiley Interscience, 1999. In addition, the preparation may include various purifications, such as column chromatography, flash chromatography, thin-layer chromatography (TLC), recrystallization, distillation, high-pressure liquid chromatography (HPLC) and the like. Also, various techniques well known in the chemical arts for the identification and quantification of chemical reaction products, such as proton and carbon-13 nuclear magnetic resonance (¹H and ¹³C NMR), infrared and ultraviolet spectroscopy (IR and UV), X-ray crystallography, elemental analysis (EA), HPLC and mass spectroscopy (MS) can be used as well. The preparation may also involve any other methods of protection and deprotection, purification and identification and quantification that are well known in the chemical arts.

Additional descriptions related to the preparation of the present CK2 inhibitors can be found in U.S. Utility application Ser. No. 11/849,230, which was filed on Aug. 31, 2007 and published as US 2009/0105233 A1 on Apr. 23, 2009. The contents of the application is hereby incorporated in reference in their entirety for all purposes.

The terms “compound(s) of the invention”, “these compounds”, “such compound(s)”, “the compound(s)”, and “the present compound(s)” refer to compounds encompassed by structural formulae disclosed herein, e.g., Formula (A), (I), (II), (III), (IV), (Ia), (Ib), (Ic), and (Id), includes any specific compounds within these formulae whose structure is disclosed herein. Compounds may be identified either by their chemical structure and/or chemical name. When the chemical structure and chemical name conflict, the chemical structure is determinative of the identity of the compound. Furthermore, the present compounds can inhibit the biological activity of a CK2 protein, and thereby is also referred to herein as an “inhibitor(s)” or “CK2 inhibitor(s)”. Compounds of Formula (A), (I), (II), (III), (IV), (Ia), (Ib), (Ic), and (Id), including any specific compounds described herein are exemplary “inhibitors”.

The present compounds may contain one or more chiral centers and/or double bonds and therefore, may exist as stereoisomers, such as double-bond isomers (i.e., geometric isomers such as E and Z), enantiomers or diastereomers. The invention includes each of the isolated stereoisomeric forms as well as mixtures of stereoisomers in varying degrees of chiral purity, including racemic mixtures and mixtures of diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The invention includes each of the isolated stereoisomeric forms as well as mixtures of stereoisomers in varying degrees of chiral purity, including racemic mixtures. It also encompasses the various diastereomers. Other structures may appear to depict a specific isomer, but that is merely for convenience, and is not intended to limit the invention to the depicted olefin isomer.

The present compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds. The term “tautomer” as used herein refers to isomers that change into one another with great ease so that they can exist together in equilibrium. For example, ketone and enol are two tautomeric forms of one compound. In another example, a substituted 1,2,4-triazole derivative may exist in at least three tautomeric forms as shown below:

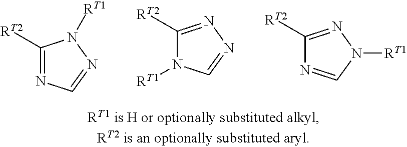

The compounds of the invention often have ionizable groups so as to be capable of preparation as salts. In that case, wherever reference is made to the compound, it is understood in the art that a pharmaceutically acceptable salt may also be used. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art. In some cases, the compounds may contain both an acidic and a basic functional group, in which case they may have two ionized groups and yet have no net charge. Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa.

“Solvate”, as used herein, means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate”. Examples of hydrate include, but are not limited to, hemihydrate, monohydrate, dihydrate, trihydrate, hexahydrate, etc. It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

The term “ester” means any ester of a present compound in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The hydrolysable esters of the present compounds are the compounds whose carboxyls are present in the form of hydrolysable ester groups. That is, these esters are pharmaceutically acceptable and can be hydrolyzed to the corresponding carboxyl acid in vivo. These esters may be conventional ones, including lower alkanoyloxyalkyl esters, e.g. pivaloyloxymethyl and 1-pivaloyloxyethyl esters; lower alkoxycarbonylalkyl esters, e.g., methoxycarbonyloxymethyl, 1-ethoxycarbonyloxyethyl, and 1-isopropylcarbonyloxyethyl esters; lower alkoxymethyl esters, e.g., methoxymethyl esters, lactonyl esters, benzofuran keto esters, thiobenzofuran keto esters; lower alkanoylaminomethyl esters, e.g., acetylaminomethyl esters. Other esters can also be used, such as benzyl esters and cyano methyl esters. Other examples of these esters include: (2,2-dimethyl-1-oxypropyloxy)methyl esters; (1RS)-1-acetoxyethyl esters, 2-[(2-methylpropyloxy)carbonyl]-2-pentenyl esters, 1-[[(1-methylethoxy)carbonyl]-oxy]ethyl esters; isopropyloxycarbonyloxyethyl esters, (5-methyl-2-oxo-1,3-dioxole-4-yl)methyl esters, 1-[[(cyclohexyloxy)carbonyl]oxy]ethyl esters; 3,3-dimethyl-2-oxobutyl esters. It is obvious to those skilled in the art that hydrolysable esters of the compounds of the present invention can be formed at free carboxyls of said compounds by using conventional methods. Representative esters include pivaloyloxymethyl esters, isopropyloxycarbonyloxyethyl esters and (5-methyl-2-oxo-1,3-dioxole-4-yl)methyl esters.

The term “prodrug” refers to a precursor of a pharmaceutically active compound wherein the precursor itself may or may not be pharmaceutically active but, upon administration, will be converted, either metabolically or otherwise, into the pharmaceutically active compound or drug of interest. For example, prodrug can be an ester, ether, or amide form of a pharmaceutically active compound. Various types of prodrug have been prepared and disclosed for a variety of pharmaceuticals. See, for example, Bundgaard, H. and Moss, J., J. Pharm. Sci. 78: 122-126 (1989). Thus, one of ordinary skill in the art knows how to prepare these prodrugs with commonly employed techniques of organic synthesis.

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C or as C1-C10 or C1-10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the backbone of the ring or chain being described.

Typically, the alkyl, alkenyl and alkynyl substituents of the invention contain 1-10C (alkyl) or 2-10C (alkenyl or alkynyl). Preferably they contain 1-8C (alkyl) or 2-8C (alkenyl or alkynyl). Sometimes they contain 1-4C (alkyl) or 2-4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCSNR₂, NRC(═NR)NR₂, NRCOOR, NRCOR, CN, C≡CR, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, C≡CR′, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group. Where two R or R′ are present on the same atom (e.g., NR₂), or on adjacent atoms that are bonded together (e.g., —NR—C(O)R), the two R or R; groups can be taken together with the atoms they are connected to to form a 5-8 membered ring, which can be substituted with C1-C4 alkyl, C1-C4 acyl, halo, C1-C4 alkoxy, and the like, and can contain an additional heteroatom selected from N, O and S as a ring member.

“Optionally substituted” as used herein indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced according to the number of available valences.

“Substituted,” when used to modify a specified group or radical, means that one or more hydrogen atoms of the specified group or radical are each, independently of one another, replaced with the same or different substituent(s).

Substituent groups useful for substituting saturated carbon atoms in the specified group or radical include, but are not limited to —R^{a}, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}s may be taken together with the nitrogen atom to which they are bonded form a 4-, 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl. As another specific example, a substituted alkyl is meant to include -alkylene-O-alkyl, -alkylene-heteroaryl, -alkylene-cycloheteroalkyl, -alkylene-C(O)OR^{b}, -alkylene-C(O)NR^{b}R^{b}, and —CH₂—CH₂—C(O)—CH₃. The one or more substituent groups, taken together with the atoms to which they are bonded, may form a cyclic ring including cycloalkyl and cycloheteroalkyl.

Similarly, substituent groups useful for substituting unsaturated carbon atoms in the specified group or radical include, but are not limited to, —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups useful for substituting nitrogen atoms in heteroalkyl and cycloheteroalkyl groups include, but are not limited to, —R^{a}, —O⁻, —OR^{b}, —SR^{b}, —S⁻, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

“Acetylene” substituents are 2-10C alkynyl groups that are optionally substituted, and are of the formula —C≡C—R^{a}, wherein R^{a} is H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl, and each R^{a} group is optionally substituted with one or more substituents selected from halo, ═O, ═N—CN, ═N—OR′, ═NR′, OR′, NR′₂, SR′, SO₂R′, SO₂NR′₂, NR′SO₂R′, NR′CONR′₂, NR′CSNR′₂, NR′C(═NR′)NR′₂, NR′COOR′, NR′COR′, CN, COOR′, CONR′₂, OOCR′, COR′, and NO₂, wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O; and wherein two R′ can be linked to form a 3-7 membered ring optionally containing up to three heteroatoms selected from N, O and S. In some embodiments, R^{a} of —C≡C—R^{a} is H or Me. Where two R or R′ are present on the same atom (e.g., NR₂), or on adjacent atoms that are bonded together (e.g., —NR—C(O)R), the two R or R; groups can be taken together with the atoms they are connected to to form a 5-8 membered ring, which can be substituted with C1-C4 alkyl, C1-C4 acyl, halo, C1-C4 alkoxy, and the like, and can contain an additional heteroatom selected from N, O and S as a ring member.

“Heteroalkyl”, “heteroalkenyl”, and “heteroalkynyl” and the like are defined similarly to the corresponding hydrocarbyl(alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical and preferred sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCSNR₂, NRC(=NR)NR₂, NRCOOR, NRCOR, CN, C≡CR, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. Where two R or R′ are present on the same atom (e.g., NR₂), or on adjacent atoms that are bonded together (e.g., —NR—C(O)R), the two R or R; groups can be taken together with the atoms they are connected to to form a 5-8 membered ring, which can be substituted with C1-C4 alkyl, C1-C4 acyl, halo, C1-C4 alkoxy, and the like, and can contain an additional heteroatom selected from N, O and S as a ring member.

The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. Thus —CH(Me)- and —C(Me)₂- may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Heteroform” as used herein refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Halo”, as used herein includes fluoro, chloro, bromo and iodo. Fluoro and chloro are often preferred.

“Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle” refers to a cyclic compound containing only carbon atoms in the ring, whereas a “heterocycle” refers to a cyclic compound comprising a heteroatom. The carbocyclic and heterocyclic structures encompass compounds having monocyclic, bicyclic or multiple ring systems. As used herein, these terms also include rings that contain a double bond or two, as long as the ring is not aromatic.

As used herein, the term “heteroatom” refers to any atom that is not carbon or hydrogen, such as nitrogen, oxygen or sulfur.

Illustrative examples of heterocycles include but are not limited to tetrahydrofuran, 1,3-dioxolane, 2,3-dihydrofuran, pyran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4b]pyridine, piperazine, pyrazine, morpholine, thiomorpholine, imidazole, imidazolidine 2,4-dione, 1,3-dihydrobenzimidazol-2-one, indole, thiazole, benzothiazole, thiadiazole, thiophene, tetrahydro thiophene 1,1-dioxide, diazepine, triazole, guanidine, diazabicyclo[2.2.1]heptane, 2,5-diazabicyclo[2.2.1]heptane, 2,3,4,4a,9,9a-hexahydro-1H-β-carboline, oxirane, oxetane, tetrahydropyran, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

As used herein, the term “inorganic substituent” refers to substituents that do not contain carbon or contain carbon bound to elements other than hydrogen (e.g., elemental carbon, carbon monoxide, carbon dioxide, and carbonate). Examples of inorganic substituents include but are not limited to nitro, halogen, azido, cyano, sulfonyls, sulfinyls, sulfonates, phosphates, etc.

The term “polar substituent” as used herein refers to any substituent having an electric dipole, and optionally a dipole moment (e.g., an asymmetrical polar substituent has a dipole moment and a symmetrical polar substituent does not have a dipole moment). Polar substituents include substituents that accept or donate a hydrogen bond, and groups that would carry at least a partial positive or negative charge in aqueous solution at physiological pH levels. In certain embodiments, a polar substituent is one that can accept or donate electrons in a non-covalent hydrogen bond with another chemical moiety.

In certain embodiments, a polar substituent is selected from a carboxy, a carboxy bioisostere or other acid-derived moiety that exists predominately as an anion at a pH of about 7 to 8 or higher. Other polar substituents include, but are not limited to, groups containing an OH or NH, an ether oxygen, an amine nitrogen, an oxidized sulfur or nitrogen, a carbonyl, a nitrile, and a nitrogen-containing or oxygen-containing heterocyclic ring whether aromatic or non-aromatic. In some embodiments, the polar substituent (represented by X) is a carboxylate or a carboxylate bioisostere.

“Carboxylate bioisostere” or “carboxy bioisostere” as used herein refers to a moiety that is expected to be negatively charged to a substantial degree at physiological pH. In certain embodiments, the carboxylate bioisostere is a moiety selected from the group consisting of:

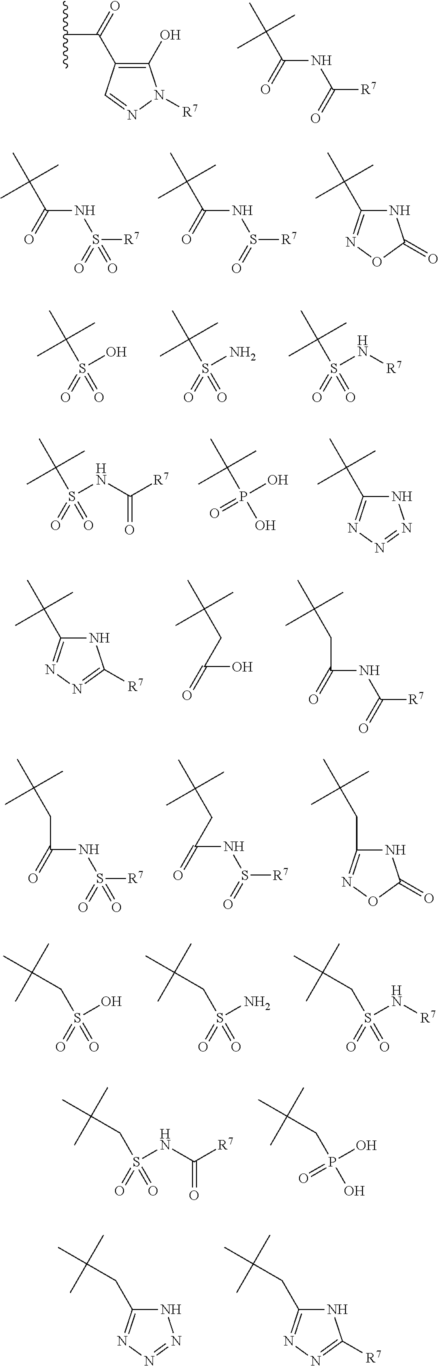

and salts of the foregoing, wherein each R⁷ is independently H or an optionally substituted member selected from the group consisting of C₁₋₁₀ alkyl, C₂₋₁₀ alkenyl, C₂₋₁₀ heteroalkyl, C₃₋₈ carbocyclic ring, and C₃₋₈ heterocyclic ring optionally fused to an additional optionally substituted carbocyclic or heterocyclic ring; or R⁷ is a C₁₋₁₀ alkyl, C₂₋₁₀ alkenyl, or C₂₋₁₀ heteroalkyl substituted with an optionally substituted C₃₋₈ carbocyclic ring or C₃₋₈ heterocyclic ring.

In certain embodiments, the polar substituent is selected from the group consisting of carboxylic acid, carboxylic ester, carboxamide, tetrazole, triazole, oxadiazole, oxothiadiazole, thiazole, aminothiazole, hydroxythiazole, and carboxymethanesulfonamide. In some embodiments of the compounds described herein, at least one polar substituent present is a carboxylic acid or a salt, or ester or a bioisostere thereof. In certain embodiments, at least one polar substituent present is a carboxylic acid-containing substituent or a salt, ester or bioisostere thereof. In the latter embodiments, the polar substituent may be a C1-C10 alkyl or C1-C10 alkenyl linked to a carboxylic acid (or salt, ester or bioisostere thereof), for example.

The term ‘solgroup’ or ‘solubility-enhancing group’ as used herein refers to a molecular fragment selected for its ability to enhance physiological solubility of a compound that has otherwise relatively low solubility. Any substituent that can facilitate the dissolution of any particular molecule in water or any biological media can serve as a solubility-enhancing group. Examples of solubilizing groups are, but are not limited to: any substituent containing a group succeptible to being ionized in water at a pH range from 0 to 14; any ionizable group succeptible to form a salt; or any highly polar substituent, with a high dipolar moment and capable of forming strong interaction with molecules of water. Examples of solubilizing groups are, but are not limited to: substitued alkyl amines, substituted alkyl alcohols, alkyl ethers, aryl amines, pyridines, phenols, carboxylic acids, tetrazoles, sulfonamides, amides, sulfonylamides, sulfonic acids, sulfinic acids, phosphates, sulfonylureas.

Suitable groups for this purpose include, for example, groups of the formula -A-(CH₂)₀₋₄-G, where A is absent, O, or NR, where R is H or Me; and G can be a carboxy group, a carboxy bioisostere, hydroxy, phosphonate, sulfonate, or a group of the formula —NR^{y}₂ or P(O)(OR^{y})₂, where each R^{y} is independently H or a C1-C4 alkyl that can be substituted with one or more (typically up to three) of these groups: NH₂, OH, NHMe, NMe₂, OMe, halo, or ═O (carbonyl oxygen); and two Ry in one such group can be linked together to form a 5-7 membered ring, optionally containing an additional heteroatom (N, O or S) as a ring member, and optionally substituted with a C1-C4 alkyl, which can itself be substituted with one or more (typically up to three) of these groups: NH₂, OH, NHMe, NMe₂, OMe, halo, or ═O (carbonyl oxygen).

Predicting Sensitivity and/or Monitoring Responsiveness of CK-2 Mediated Diseases to Treatment with Therapeutic Combinations Comprising CK2 Inhibitors

In addition to the above-described embodiments, the present invention also provides biomarkers for predicting the sensitivity and/or monitoring the response of a CK2-mediated disease, such as a proliferative disorder and/or an inflammatory disorder, with CK2 inhibitors when used in combination with additional therapeutic agents.

In one aspect, the present invention provides biomarkers that are useful for predicting the sensitivity and/or responsiveness of a subject or system to treatment with a CK2 inhibitor when used in combination with additional therapeutic agents, such as anti-cancer, anti-inflammatory, anti-infective agents, as well as therapeutics for the treatment of pain (e.g. analgesics) and autoimmune disorders. Thus, in one embodiment, the biomarkers and associated methods of measuring said biomarkers can be used to select an individual subject or a population of subjects for treatment with a particular therapeutic combination comprising a CK2 inhibitor. The invention also relates to the use of these biomarkers to monitor or predict the outcome of treatment in subjects being administered a therapeutic combination comprising a CK2 inhibitor.

As described herein, biomarkers useful for predicting the sensitivity and/or monitoring the responsiveness of a CK2-mediated disease to treatment with a therapeutic combination comprising a CK2 inhibitor include the mRNA expression and/or polypeptide levels (i.e., the protein expression) of IL-6, IL-8, HIF-1α, VEGF, CK2α and/or CK2α′ subunits, CK2β, and the level of phosphorylated Akt serine 129 (p-Akt S129), alone or relative to total Akt polypeptide (i.e., the normalized level of p-Akt S129). Additional biomarkers include the level of phosphorylated Akt serine 473 (p-Akt S473), alone or relative to total Akt polypeptide (i.e., the normalized level of p-Akt S473), the level of phosphorylated p21 threonine 145 (p-p21 T145), alone or relative to total p21 polypeptide (i.e., the normalized level of p-p21 T145), the level of phosphorylated nuclear factor-κB (NF-κB) serine 529 (p-NF-κB S529), alone or relative to total NF-κB polypeptide (i.e., the normalized level of p-NF-κK S529), the level of phosphorylated STAT3 tyrosine 705 (p-STAT3 Y705), alone or relative to total STAT3 polypeptide (i.e., the normalized level of p-STAT3 Y705), or the level of phosphorylated JAK2 tyrosine 1007/1008 (p-JAK2 Y1007/1008), alone or relative to total JAK2 polypeptide (i.e., the normalized level of p-JAK2 Y1007/1008).

In one embodiment, the therapeutic combination comprises a CK2 inhibitor and one additional therapeutic agent. In alternative embodiments, the therapeutic composition comprises a CK2 inhibitor and two, three, four, five, or more additional therapeutic agents.

In one embodment, the additional therapeutic agent is an anti-cancer agent. Anti-cancer agents used in combination with the CK2 inhibitors of the present application may include agents selected from any of the classes known to those of ordinary skill in the art, including, for example, alkylating agents, anti-metabolites, plant alkaloids and terpenoids (e.g., taxanes), topoisomerase inhibitors, anti-tumor antibiotics, hormonal therapies, molecular targeted agents, and the like. Generally such an anticancer agent is an alkylating agent, an anti-metabolite, a vinca alkaloid, a taxane, a topoisomerase inhibitor, an anti-tumor antibiotic, a tyrosine kinase inhibitor, an immunosuppressive macrolide, an Akt inhibitor, an HDAC inhibitor, an Hsp90 inhibitor, an mTOR inhibitor, a PI3K/mTOR inhibitor, or a PI3K inhibitor. Commonly, an anticancer agent is selected from the group consisting of an Akt inhibitor, an HDAC inhibitor, an Hsp90 inhibitor, an mTOR inhibitor, a PI3K/mTOR inhibitor, a PI3K inhibitor, and a monoclonal antibody targeting a tumor/cancer antigen; alternately an anticancer agent is selected from the group consisting of an Akt inhibitor, an HDAC inhibitor, an Hsp90 inhibitor, an mTOR inhibitor, a PI3K/mTOR inhibitor and a PI3K inhibitor.

Alkylating agents include (a) alkylating-like platinum-based chemotherapeutic agents such as cisplatin, carboplatin, nedaplatin, oxaliplatin, satraplatin, and (SP-4-3)-(cis)-amminedichloro-[2-methylpyridine] platinum(II); (b) alkyl sulfonates such as busulfan; (c) ethyleneimine and methylmelamine derivatives such as altretamine and thiotepa; (d) nitrogen mustards such as chlorambucil, cyclophosphamide, estramustine, ifosfamide, mechlorethamine, trofosamide, prednimustine, melphalan, and uramustine; (e) nitrosoureas such as carmustine, lomustine, fotemustine, nimustine, ranimustine and streptozocin; (f) triazenes and imidazotetrazines such as dacarbazine, procarbazine, temozolamide, and temozolomide.

Anti-metabolites include (a) purine analogs such as fludarabine, cladribine, chlorodeoxyadenosine, clofarabine, mercaptopurine, pentostatin, and thioguanine; (b) pyrimidine analogs such as fluorouracil, gemcitabine, capecitabine, cytarabine, azacitidine, edatrexate, floxuridine, and troxacitabine; (c) antifolates, such as methotrexate, pemetrexed, raltitrexed, and trimetrexate. Anti-metabolites also include thymidylate synthase inhibitors, such as fluorouracil, raltitrexed, capecitabine, floxuridine and pemetrexed; and ribonucleotide reductase inhibitors such as claribine, clofarabine and fludarabine.

Plant alkaloid and terpenoid derived agents include mitotic inhibitors such as the vinca alkaloids vinblastine, vincristine, vindesine, and vinorelbine; and microtubule polymer stabilizers such as the taxanes, including, but not limited to paclitaxel, docetaxel, larotaxel, ortataxel, and tesetaxel.

Topoisomerase inhibitors include topoisomerase I inhibitors such as camptothecin, topotecan, irinotecan, rubitecan, and belotecan; and topoisomerase II inhibitors such as etoposide, teniposide, and amsacrine.

Anti-tumor antibiotics include (a) anthracyclines such as daunorubicin (including liposomal daunorubicin), doxorubicin (including liposomal doxorubicin), epirubicin, idarubicin, and valrubicin; (b) streptomyces-related agents such as bleomycin, actinomycin, mithramycin, mitomycin, porfiromycin; and (c) anthracenediones, such as mitoxantrone and pixantrone. Anthracyclines have three mechanisms of action: intercalating between base pairs of the DNA/RNA strand; inhibiting topoiosomerase II enzyme; and creating iron-mediated free oxygen radicals that damage the DNA and cell membranes. Anthracyclines are generally characterized as topoisomerase II inhibitors.

Hormonal therapies include (a) androgens such as fluoxymesterone and testolactone; (b) antiandrogens such as bicalutamide, cyproterone, flutamide, and nilutamide; (c) aromatase inhibitors such as aminoglutethimide, anastrozole, exemestane, formestane, and letrozole; (d) corticosteroids such as dexamethasone and prednisone; (e) estrogens such as diethylstilbestrol; (f) antiestrogens such as fulvestrant, raloxifene, tamoxifen, and toremifene; (g) LHRH agonists and antagonists such as buserelin, goserelin, leuprolide, and triptorelin; (h) progestins such as medroxyprogesterone acetate and megestrol acetate; and (i) thyroid hormones such as levothyroxine and liothyronine.

Molecular targeted agents include (a) receptor tyrosine kinase (‘RTK’) inhibitors, such as inhibitors of EGFR, including erlotinib, gefitinib, and neratinib; inhibitors of VEGFR including vandetanib, semaxinib, and cediranib; and inhibitors of PDGFR; further included are RTK inhibitors that act at multiple receptor sites such as lapatinib, which inhibits both EGFR and HER2, as well as those inhibitors that act at each of C-kit, PDGFR and VEGFR, including but not limited to axitinib, sunitinib, sorafenib and toceranib; also included are inhibitors of BCR-ABL, c-kit and PDGFR, such as imatinib; (b) FKBP binding agents, such as an immunosuppressive macrolide antibiotic, including bafilomycin, rapamycin (sirolimus) and everolimus; (c) gene therapy agents, antisense therapy agents, and gene expression modulators such as the retinoids and rexinoids, e.g. adapalene, bexarotene, trans-retinoic acid, 9-cis-retinoic acid, and N-(4-hydroxyphenyl)retinamide; (d) phenotype-directed therapy agents, including monoclonal antibodies such as alemtuzumab, bevacizumab, cetuximab, ibritumomab tiuxetan, rituximab, and trastuzumab; (e) immunotoxins such as gemtuzumab ozogamicin; (f) radioimmunoconjugates such as 131I-tositumomab; and (g) cancer vaccines.

Monoclonal antibodies include, but are not limited to, murine, chimeric, or partial or fully humanized monoclonal antibodies. Such therapeutic antibodies include, but are not limited to antibodies directed to tumor or cancer antigens either on the cell surface or inside the cell. Such therapeutic antibodies also include, but are not limited to antibodies directed to targets or pathways directly or indirectly associated with CK2. Therapeutic antibodies may further include, but are not limited to antibodies directed to targets or pathways that directly interact with targets or pathways associated with the compounds of the present invention. In one variation, therapeutic antibodies include, but are not limited to anticancer agents such as Abagovomab, Adecatumumab, Afutuzumab, Alacizumab pegol, Alemtuzumab, Altumomab pentetate, Anatumomab mafenatox, Apolizumab, Bavituximab, Belimumab, Bevacizumab, Bivatuzumab mertansine, Blinatumomab, Brentuximab vedotin, Cantuzumab mertansine, Catumaxomab, Cetuximab, Citatuzumab bogatox, Cixutumumab, Clivatuzumab tetraxetan, Conatumumab, Dacetuzumab, Detumomab, Ecromeximab, Edrecolomab, Elotuzumab, Epratuzumab, Ertumaxomab, Etaracizumab, Farletuzumab, Figitumumab, Fresolimumab, Galiximab, Glembatumumab vedotin, Ibritumomab tiuxetan, Intetumumab, Inotuzumab ozogamicin, Ipilimumab, Iratumumab, Labetuzumab, Lexatumumab, Lintuzumab, Lucatumumab, Lumiliximab, Mapatumumab, Matuzumab, Milatuzumab, Mitumomab, Nacolomab tafenatox, Naptumomab estafenatox, Necitumumab, Nimotuzumab, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Sibrotuzumab, Tacatuzumab tetraxetan, Taplitumomab paptox, Tenatumomab, Ticilimumab, Tigatuzumab, Tositumomab, Trastuzumab, Tremelimumab, Tucotuzumab celmoleukin, Veltuzumab, Volociximab, Votumumab, Zalutumumab, and Zanolimumab. In some embodiments, such therapeutic antibodies include, alemtuzumab, bevacizumab, cetuximab, daclizumab, gemtuzumab, ibritumomab tiuxetan, pantitumumab, rituximab, tositumomab, and trastuzumab; in other embodiments, such monoclonal antibodies include alemtuzumab, bevacizumab, cetuximab, ibritumomab tiuxetan, rituximab, and trastuzumab; alternately, such antibodies include daclizumab, gemtuzumab, and pantitumumab. In yet another embodiment, therapeutic antibodies useful in the treatment of infections include but are not limited to Afelimomab, Efungumab, Exbivirumab, Felvizumab, Foravirumab, Ibalizumab, Libivirumab, Motavizumab, Nebacumab, Pagibaximab, Palivizumab, Panobacumab, Rafivirumab, Raxibacumab, Regavirumab, Sevirumab, Tefibazumab, Tuvirumab, and Urtoxazumab. In a further embodiment, therapeutic antibodies can be useful in the treatment of inflammation and/or autoimmune disorders, including, but are not limited to, Adalimumab, Atlizumab, Atorolimumab, Aselizumab, Bapineuzumab, Basiliximab, Benralizumab, Bertilimumab, Besilesomab, Briakinumab, Canakinumab, Cedelizumab, Certolizumab pegol, Clenoliximab, Daclizumab, Denosumab, Eculizumab, Edobacomab, Efalizumab, Erlizumab, Fezakinumab, Fontolizumab, Fresolimumab, Gantenerumab, Gavilimomab, Golimumab, Gomiliximab, Infliximab, Inolimomab, Keliximab, Lebrikizumab, Lerdelimumab, Mepolizumab, Metelimumab, Muromonab-CD3, Natalizumab, Ocrelizumab, Odulimomab, Omalizumab, Otelixizumab, Pascolizumab, Priliximab, Reslizumab, Rituximab, Rontalizumab, Rovelizumab, Ruplizumab, Sifalimumab, Siplizumab, Solanezumab, Stamulumab, Talizumab, Tanezumab, Teplizumab, Tocilizumab, Toralizumab, Ustekinumab, Vedolizumab, Vepalimomab, Visilizumab, Zanolimumab, and Zolimomab aritox. In yet another embodiment, such therapeutic antibodies include, but are not limited to adalimumab, basiliximab, certolizumab pegol, eculizumab, efalizumab, infliximab, muromonab-CD3, natalizumab, and omalizumab. Alternately the therapeutic antibody can include abciximab or ranibizumab. Generally a therapeutic antibody is non-conjugated, or is conjugated with a radionuclide, cytokine, toxin, drug-activating enzyme or a drug-filled liposome.

Akt inhibitors include 1L6-Hydroxymethyl-chiro-inositol-2-(R)-2-O-methyl-3-O-octadecyl-sn-glycerocarbonate, SH-5 (Calbiochem Cat. No. 124008), SH-6 (Calbiochem Cat. No. Cat. No. 124009), Calbiochem Cat. No. 124011, Triciribine (NSC 154020, Calbiochem Cat. No. 124012), 10-(4′-(N-diethylamino)butyl)-2-chlorophenoxazine, Cu(II)Cl₂(3-Formylchromone thiosemicarbazone), 1,3-dihydro-1-(1-((4-(6-phenyl-1H-imidazo[4,5-g]quinoxalin-7-yl)phenyl)methyl)-4-piperidinyl)-2H-benzimidazol-2-one, GSK690693 (4-(2-(4-amino-1,2,5-oxadiazol-3-yl)-1-ethyl-7-{[(3S)-3-piperidinylmethyl]oxy}-1H-imidazo[4,5-c]pyridin-4-yl)-2-methyl-3-butyn-2-ol), SR13668 ((2,10-dicarbethoxy-6-methoxy-5,7-dihydro-indolo[2,3-b]carbazole), GSK2141795, Perifosine, GSK21110183, XL418, XL147, PF-04691502, BEZ-235 [2-Methyl-2-[4-(3-methyl-2-oxo-8-quinolin-3-yl-2,3-dihydro-imidazo[4,5-c]quinolin-1-yl)-phenyl]-propionitrile], PX-866 ((acetic acid (1S,4E,10R,11R,13S,14R)-[4-diallylaminomethylene-6-hydroxy-1-methoxymethyl-10,13-dimethyl-3,7,17-trioxo-1,3,4,7,10,11,12,13,14,15,16,17-dodecahydro-2-oxa-cyclopenta[a]phenanthren-11-yl ester)), D-106669, CAL-101, GDC0941 (2-(1H-indazol-4-yl)-6-(4-methanesulfonyl-piperazin-1-ylmethyl)-4-morpholin-4-yl-thieno[3,2-d]pyrimidine), SF1126, SF1188, SF2523, TG100-115 [3-[2,4-diamino-6-(3-hydroxyphenyl)pteridin-7-yl]phenol]. A number of these inhibitors, such as, for example, BEZ-235, PX-866, D 106669, CAL-101, GDC0941, SF1126, SF2523 are also identified in the art as PI3K/mTOR inhibitors; additional examples, such as PI-103 [3-[4-(4-morpholinylpyrido[3′,2′:4,5]furo[3,2-d]pyrimidin-2-yl]phenol hydrochloride] are well-known to those of skill in the art. Additional well-known PI3K inhibitors include LY294002 [2-(4-morpholinyl)-8-phenyl-4H-1-benzopyran-4-one] and wortmannin. mTOR inhibitors known to those of skill in the art include temsirolimus, deforolimus, sirolimus, everolimus, zotarolimus, and biolimus A9. A representative subset of such inhibitors includes temsirolimus, deforolimus, zotarolimus, and biolimus A9.

HDAC inhibitors include (i) hydroxamic acids such as Trichostatin A, vorinostat (suberoylanilide hydroxamic acid (SAHA)), panobinostat (LBH589) and belinostat (PXD101) (ii) cyclic peptides, such as trapoxin B, and depsipeptides, such as romidepsin (NSC 630176), (iii) benzamides, such as MS-275 (3-pyridylmethyl-N-{4-[(2-aminophenyl)-carbamoyl]-benzyl}-carbamate), CI994 (4-acetylamino-N-(2aminophenyl)-benzamide) and MGCD0103 (N-(2-aminophenyl)-4-((4-(pyridin-3-yl)pyrimidin-2-ylamino)methyl)benzamide), (iv) electrophilic ketones, (v) the aliphatic acid compounds such as phenylbutyrate and valproic acid.

Hsp90 inhibitors include benzoquinone ansamycins such as geldanamycin, 17-DMAG (17-Dimethylamino-ethylamino-17-demethoxygeldanamycin), tanespimycin (17-AAG, 17-allylamino-17-demethoxygeldanamycin), ECS, retaspimycin (IPI-504, 18,21-didehydro-17-demethoxy-18,21-dideoxo-18,21-dihydroxy-17-(2-propenylamino)-geldanamycin), and herbimycin; pyrazoles such as CCT 018159 (4-[4-(2,3-dihydro-1,4-benzodioxin-6-yl)-5-methyl-1H-pyrazol-3-yl]-6-ethyl-1,3-benzenediol); macrolides, such as radicocol; as well as BIIB021 (CNF2024), SNX-5422, STA-9090, and AUY922.

Miscellaneous agents include altretamine, arsenic trioxide, gallium nitrate, hydroxyurea, levamisole, mitotane, octreotide, procarbazine, suramin, thalidomide, lenalidomide, photodynamic compounds such as methoxsalen and sodium porfimer, and proteasome inhibitors such as bortezomib.

Biologic therapy agents include: interferons such as interferon-α2a and interferon-α2b, and interleukins such as aldesleukin, denileukin diftitox, and oprelvekin.

In addition to anti-cancer agents intended to act against cancer cells, combination therapies including the use of protective or adjunctive agents, including: cytoprotective agents such as armifostine, dexrazonxane, and mesna, phosphonates such as parmidronate and zoledronic acid, and stimulating factors such as epoetin, darbeopetin, filgrastim, PEG-filgrastim, and sargramostim, are also envisioned.

In another embodment, the additional therapeutic agent is an anti-inflammatory agent. Anti-inflammatory agents used in combination with the CK2 inhibitors of the present application may include agents selected from glucocorticoids, NSAIDs, coxibs, corticosteroids, analgesics, inhibitors of 5-lipoxygenase, inhibitors of 5-lipoxygenase activating protein, and leukotriene receptor antagonists. Examples of nonsteroidal anti-inflammatory agents include, but are not limited to ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, diclofenac, ketorolac, aspirin, bextra, celebrex, vioxx and acetominophen. In one embodiment, anti-inflammatory agents are monoclonal antibodies. In another embodiment, anti-inflammatory agents are monoclonal antibodies targeting at receptors or antigens directly or indirectly associated with inflammation. In another embodiment, anti-inflammatory agents are monoclonal antibodies targeting CK2 kinase or CK2-regulated pathways. In yet another embodiment, anti-inflammatory agents include, but are not limited to Adalimumab, Atlizumab, Atorolimumab, Aselizumab, Bapineuzumab, Basiliximab, Benralizumab, Bertilimumab, Besilesomab, Briakinumab, Canakinumab, Cedelizumab, Certolizumab pegol, Clenoliximab, Daclizumab, Denosumab, Eculizumab, Edobacomab, Efalizumab, Erlizumab, Fezakinumab, Fontolizumab, Fresolimumab, Gantenerumab, Gavilimomab, Golimumab, Gomiliximab, Infliximab, Inolimomab, Keliximab, Lebrikizumab, Lerdelimumab, Mepolizumab, Metelimumab, Muromonab-CD3, Natalizumab, Ocrelizumab, Odulimomab, Omalizumab, Otelixizumab, Pascolizumab, Priliximab, Reslizumab, Rituximab, Rontalizumab, Rovelizumab, Ruplizumab, Sifalimumab, Siplizumab, Solanezumab, Stamulumab, Talizumab, Tanezumab, Teplizumab, Tocilizumab, Toralizumab, Ustekinumab, Vedolizumab, Vepalimomab, Visilizumab, Zanolimumab, and Zolimomab aritox.

In another embodment, the additional therapeutic agent is an anti-infective agent. Anti-infective agents used in combination with the CK2 inhibitors of the present application include those agents known in the art to treat viral, fungal, parasitic or bacterial infections. The term, “antibiotic,” as used herein, refers to a chemical substance that inhibits the growth of, or kills, microorganisms. Encompassed by this term are antibiotic produced by a microorganism, as well as synthetic antibiotics known in the art. Antibiotics include, but are not limited to, clarithromycin, ciprofloxacin, and metronidazole. In one embodiment, antiinfection agents are monoclonal antibodies directed to antigens associated with infectious agents or microorganisms. Non-limiting examples of monoclonal antibodies effective in the treatment of infections include Afelimomab, Efungumab Exbivirumab, Felvizumab, Foravirumab, Ibalizumab, Libivirumab, Motavizumab, Nebacumab, Pagibaximab, Palivizumab, Panobacumab, Rafivirumab, Raxibacumab, Regavirumab, Sevirumab, Tefibazumab, Tuvirumab, and Urtoxazumab.

In another embodment, the additional therapeutic agent is an immunotherapeutic agent useful for the treatment of pain, inflammation, infection and/or autoimmune disorders. Such agents used in combination with the CK2 inhibitors of the present application include include but are not limited to microorganism or bacterial components (e.g., muramyl dipeptide derivative, Picibanil), polysaccharides having immunity potentiating activity (e.g., lentinan, schizophyllan, krestin), cytokines obtained by genetic engineering techniques (e.g., interferon, interleukin (IL)), colony stimulating factors (e.g., G-CSF (Filgrastim/Pegfilgrastim, Lenograstim), GM-CSF (Molgramostim, Sargramostim), SCF (Ancestim), and erythropoietin) and the like. Monoclonal antibodies that have such therapeutic effects include, but are not limited to Adalimumab, Atlizumab, Atorolimumab, Aselizumab, Bapineuzumab, Basiliximab, Benralizumab, Bertilimumab, Besilesomab, Briakinumab, Canakinumab, Cedelizumab, Certolizumab pegol, Clenoliximab, Daclizumab, Denosumab, Eculizumab, Edobacomab, Efalizumab, Erlizumab, Fezakinumab, Fontolizumab, Fresolimumab, Gantenerumab, Gavilimomab, Golimumab, Gomiliximab, Infliximab, Inolimomab, Keliximab, Lebrikizumab, Lerdelimumab, Mepolizumab, Metelimumab, Muromonab-CD3, Natalizumab, Ocrelizumab, Odulimomab, Omalizumab, Otelixizumab, Pascolizumab, Priliximab, Reslizumab, Rituximab, Rontalizumab, Rovelizumab, Ruplizumab, Sifalimumab, Siplizumab, Solanezumab, Stamulumab, Talizumab, Tanezumab, Teplizumab, Tocilizumab, Toralizumab, Ustekinumab, Vedolizumab, Vepalimomab, Visilizumab, Zanolimumab, and Zolimomab aritox.

### EXAMPLES

The following examples illustrate but do not limit the invention.

### Example 1

### Phase I Clinical Study with CX-4945

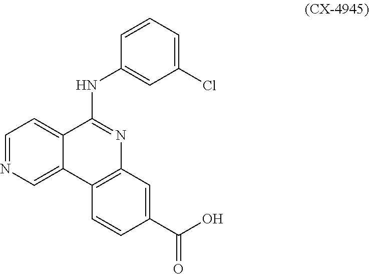

CX-4945 demonstrated single-agent potency in suppressing xenograft tumor growth with a wide therapeutic window pre-clinically. A Phase I study was undertaken to determine the maximum tolerated dose (MTD) and dose limiting toxicities (DLTs), to characterize the pharmacokinetics (PKs), and to study the pharmacodynamic effects of CX-4945.

### Procedure:

Eligible patients with advanced solid tumors, Castleman's disease or multiple myeloma with progressive disease, or for whom there are no available standard therapies, receive CX-4945 in successive dose cohorts at: 90, 160, 300, 460, 700 and 1000 mg per dose. Oral doses are administered twice daily for twenty-one consecutive days of a four week cycle. Therapy is continued in consenting patients until signs of intolerance to CX-4945 are observed, or there is evidence of advancing disease. Response by RECIST is determined after every 2 cycles. Serial blood and plasma samples are collected on the first and final dosing days of Cycle 1 (i.e., Day 1 and Day 21) for pharmacokinetic analysis and for pharmacodynamic biomarker evaluations (specifically, total and phosphorylated forms of p21 and Akt).

An additional set of patients, with the same eligibility criteria receive CX-4945 in successive dose cohorts at: 300, 500, 600 and 800 mg per dose. Oral doses are administered four times daily for twenty-one consecutive days of a four week cycle. Therapy is continued in consenting patients until signs of intolerance to CX-4945 are observed, or there is evidence of advancing disease. Response by RECIST is determined after every 2 cycles. Serial blood and plasma samples are collected on the first and eighth dosing days of Cycle 1 (i.e., Day 1 and Day 8) for pharmacokinetic analysis and for pharmacodynamic biomarker evaluations (specifically, total and phosphorylated forms of p21 and Akt).

A laser scanning cytometry method was developed and validated to quantify the phosphorylation of p21 and Akt in cells, and to characterize these substrates in circulating blood cells and circulating tumor cells (CTC) collected from patients undergoing treatment with CK2 inhibitors, such as CX-4945.

### Summary of Results:

Thirty-six patients with advanced solid tumors (3-4 patients per cohort, from six separate dose cohorts) received oral doses of CX-4945, and all patients in the study participated in collection of PBMCs. Beginning in patients in Cohort 3, biomarkers demonstrated changes in their profile concurrently with inhibition of CK2.

### Route and Schedule of Administration:

Patients in Cohorts 1-6 were dosed twice daily (BID) with oral capsules. Cohort 1 received 90 mg of CX-4945 BID. Cohort 2 received 160 mg of CX-4945 BID. Cohort 3 received 300 mg of CX-4945 BID. Cohort 4 received 460 mg of CX-4945 BID. Cohort 5 received 700 mg of CX-4945 BID. Cohort 6 received 1000 mg of CX-4945 BID.

Patients in Cohorts 7-9 were dosed four times daily (QID) with oral capsules. Cohort 7 received 300 mg of CX-4945 QID. Cohort 8 received 500 mg of CX-4945 QID. Cohort 9 received 600 mg of CX-4945 QID.

### Biomarker Analysis

To identify biomarkers useful for measuring CK2 inhibition, whole blood samples were collected at pre-treatment, 4 hours and 8 hours following the first dose of CX-4945 on Day 1 and Day 21. Plasma samples were also collected at these time points for quantification of IL-6 and IL-8, and changes in serum IL-6 and IL-8 levels following 21 days of treatment with CX-4945 were determined.

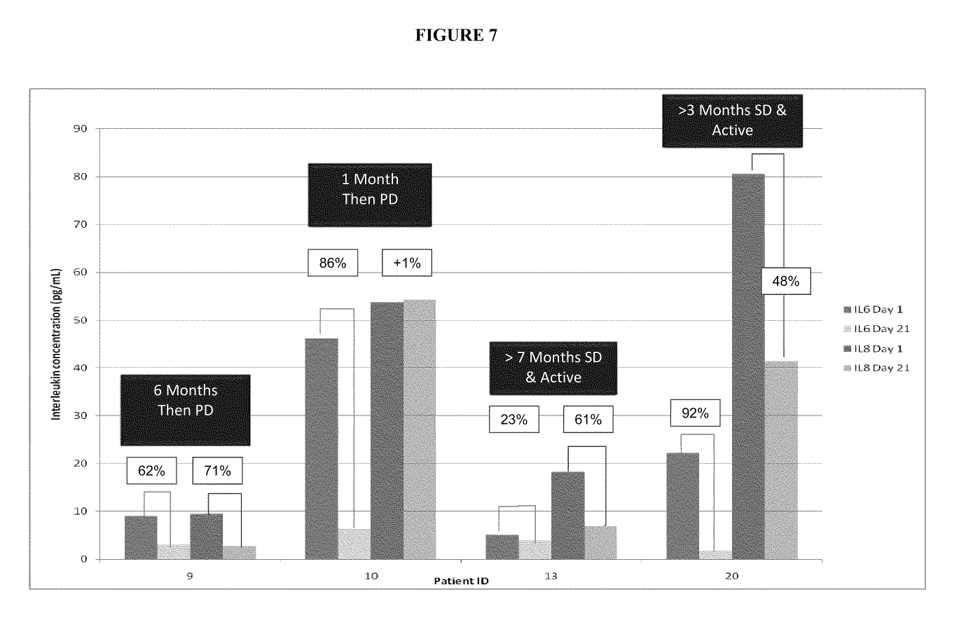

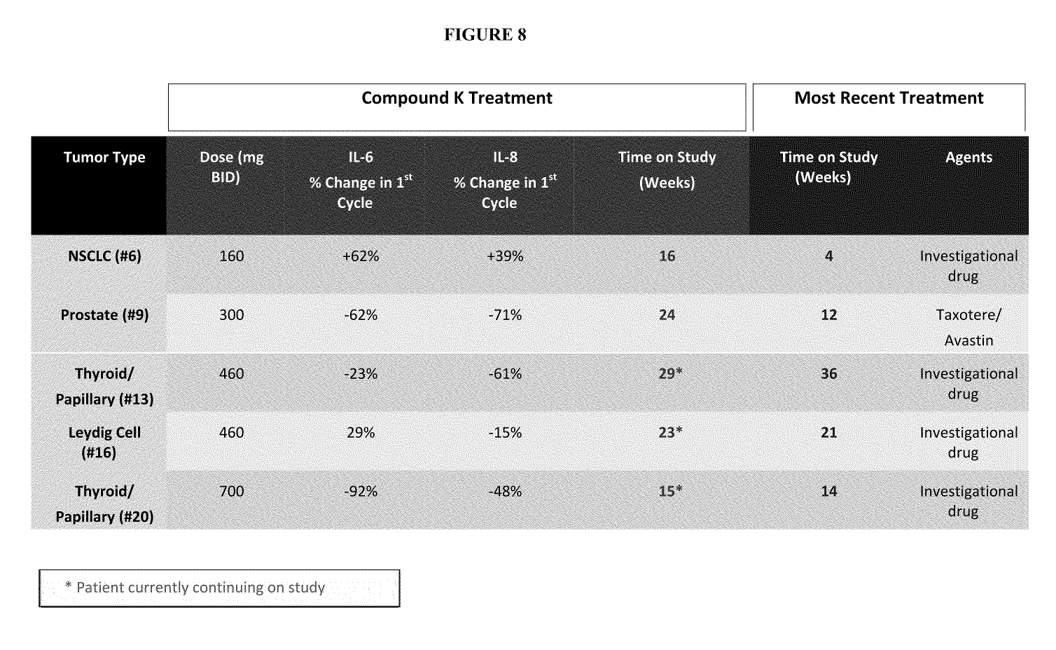

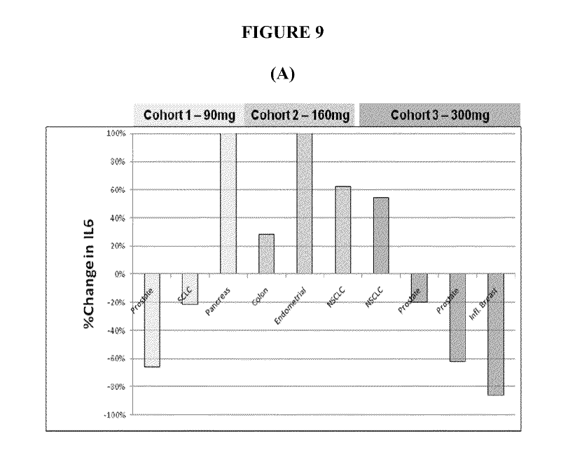

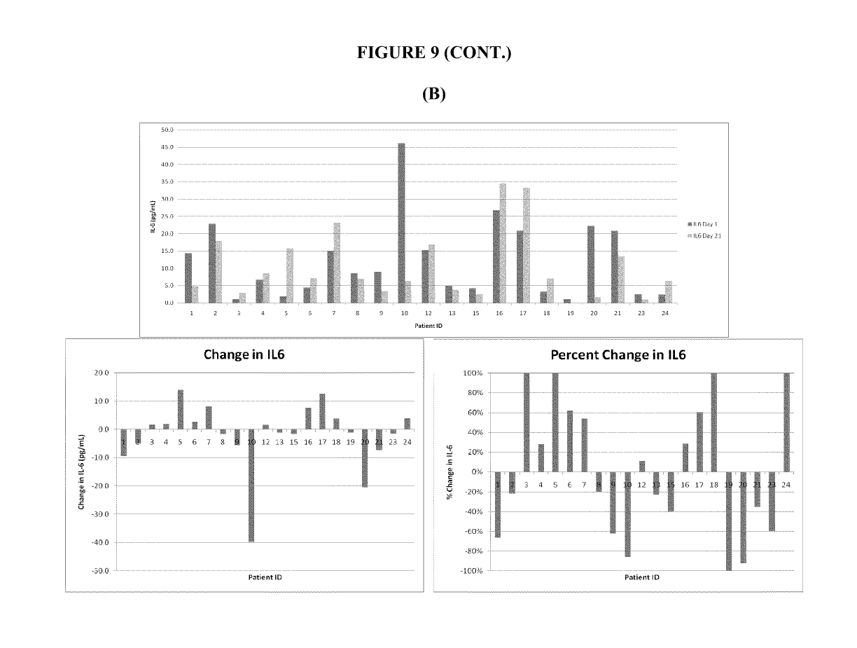

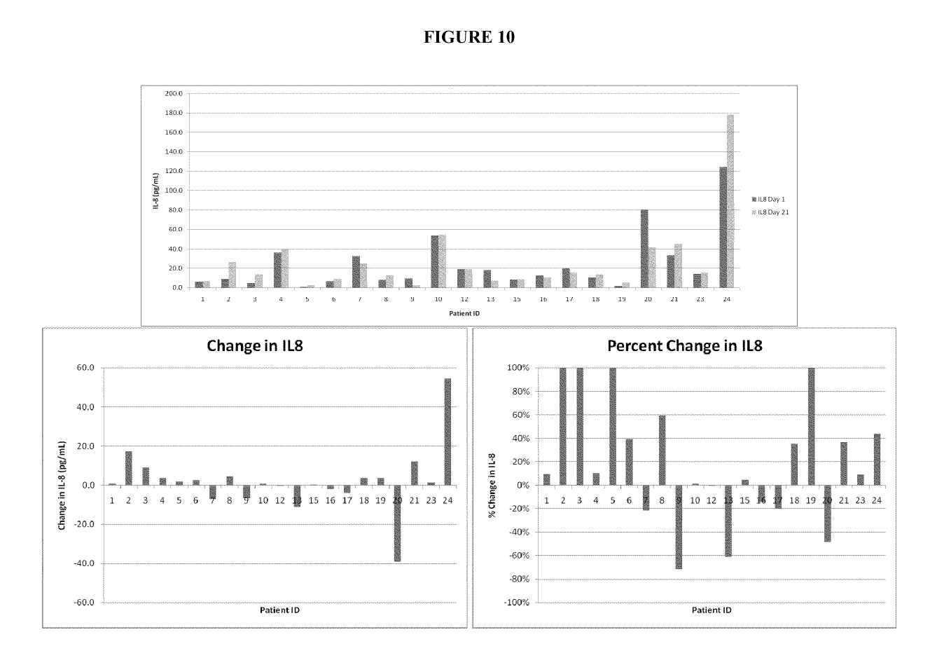

As seen in FIG. 7, IL-6 levels were significantly reduced in three patients (#9, #10, #20) and IL-8 levels were significantly reduced in three patients (#9, #13, #20). The percent change in IL-6 and IL-8 in patients undergoing treatment with Compound K (CX-4945) was determined for patients having NSCLC (#6), prostate (#9), thyroid/papillary (#13, #20) and Leydig cell tumors (#16). IL-6 levels were significantly reduced in two patients (#9, #20, with a smaller reduction in #13) and IL-8 levels were significantly reduced in three patients (#9, #13, #20). A reduction in IL-6 and IL-8 levels after 21 days of treatment was associated with the appearance of stable disease as evidenced from increased time on treatment (FIG. 8). As shown in FIGS. 9A and B. a marked reduction in serum IL-6 levels in inflammatory breast cancer (IBC) and prostate cancer patients was observed after 21 days of dosing. As shown in FIG. 10, IL-8 levels were reduced significantly in patients with prostate, thyroid/papillary, and Leydig cell tumors.

In addition, PBMCs were isolated to analyze p21 Total, p21-T145, Akt Total, Akt-T129, and Akt-S473 at time 0, 4 and 8 hours post dose on Day 1 and Day 21. PBMCs were analyzed as a whole and also separated into phenotypes (CD19, CD45). For each time point, the ratio of p21-T145/Total p21, Akt-S129/Total Akt, and Akt-S473/Total Akt was calculated.

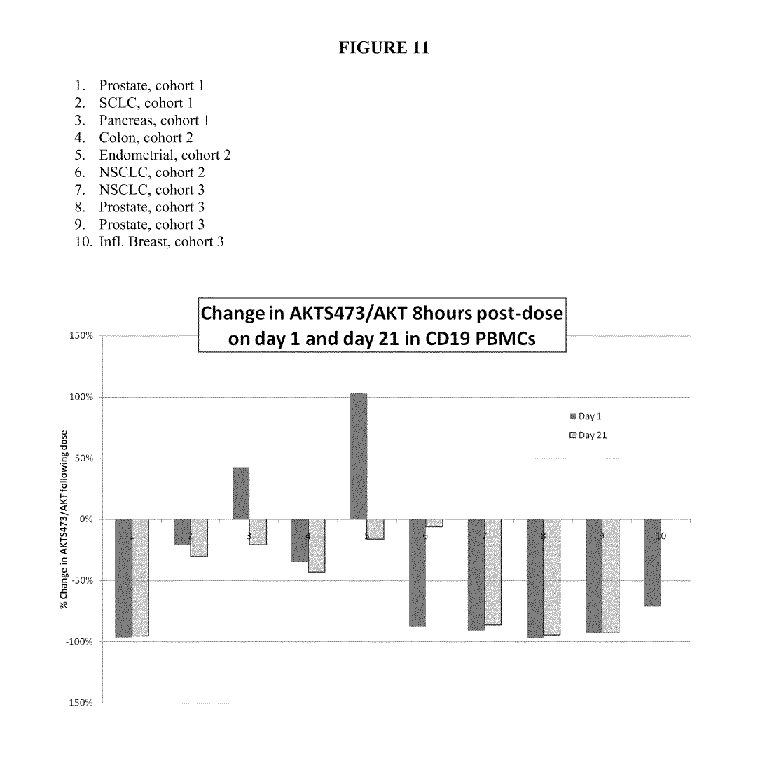

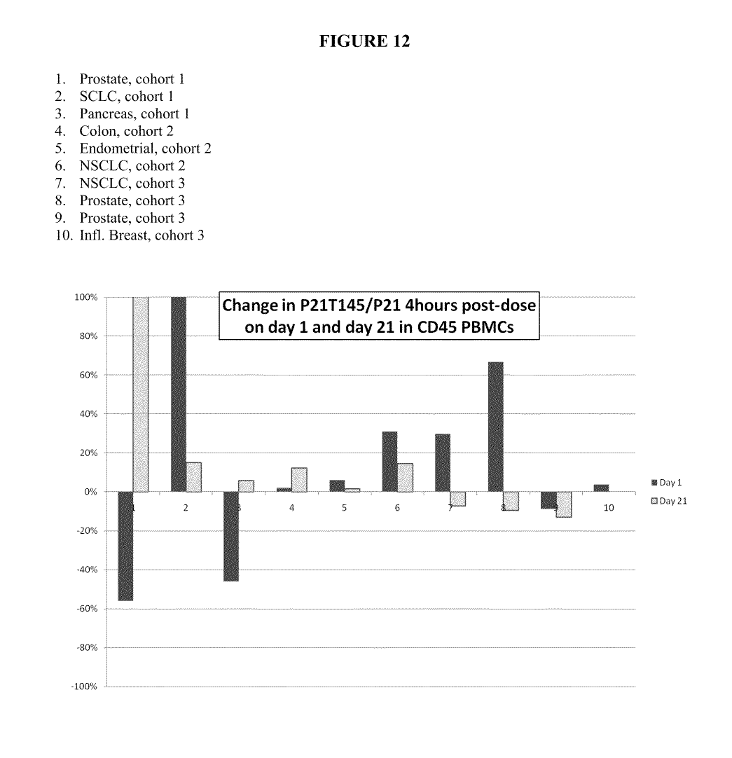

The change in the ratio of p-Akt S473 to total Akt at 8 hours post-dose on day 1 and day 21 in CD19 PBMCs for cohorts 1-3 is shown in FIG. 11. The change in the ratio of p-p21 T145 to total p21 at 4 hours post-dose on day 1 and day 21 in CD45 PBMCs is shown in FIG. 12.

In addition, PBMCs were isolated to analyze, p-p21-T145, p-Akt-S129, and p-Akt-S473 at time 0, 4 and 8 hours post dose on Day 1 and Day 21 for the BID dosing schedule and at time 0, 2, 4 and 6 hours post dose on Day 1 and Day 8 for the QID dosing schedule. PBMCs were analyzed as a whole and also separated into phenotypes (CD19, CD45).

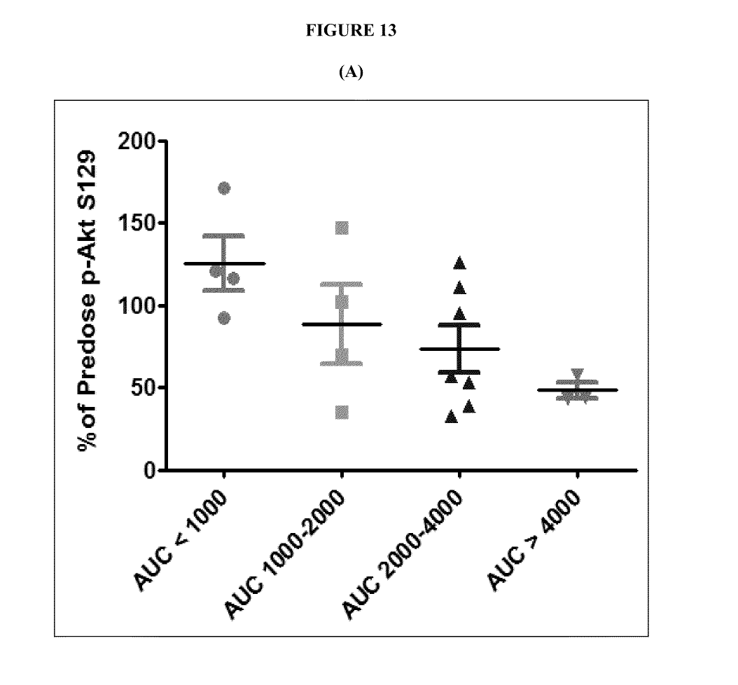

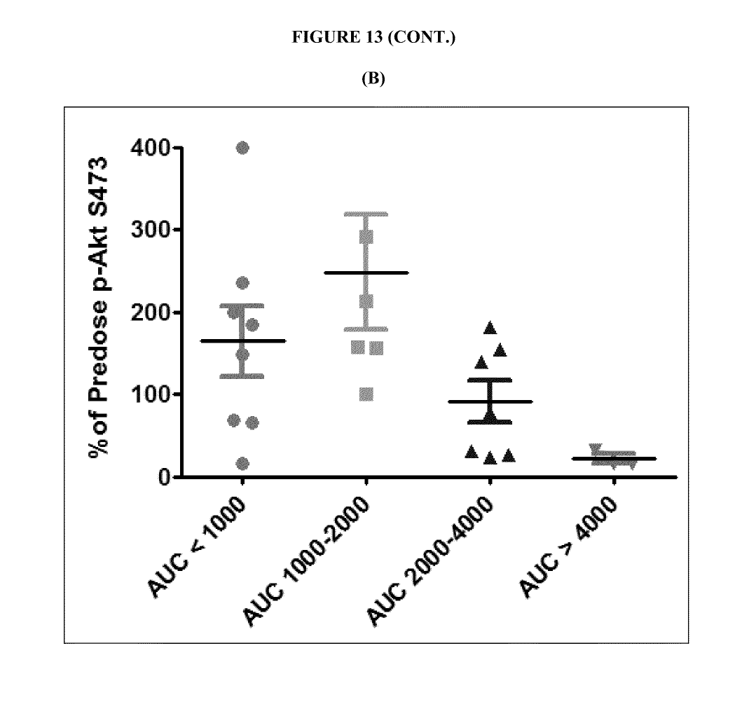

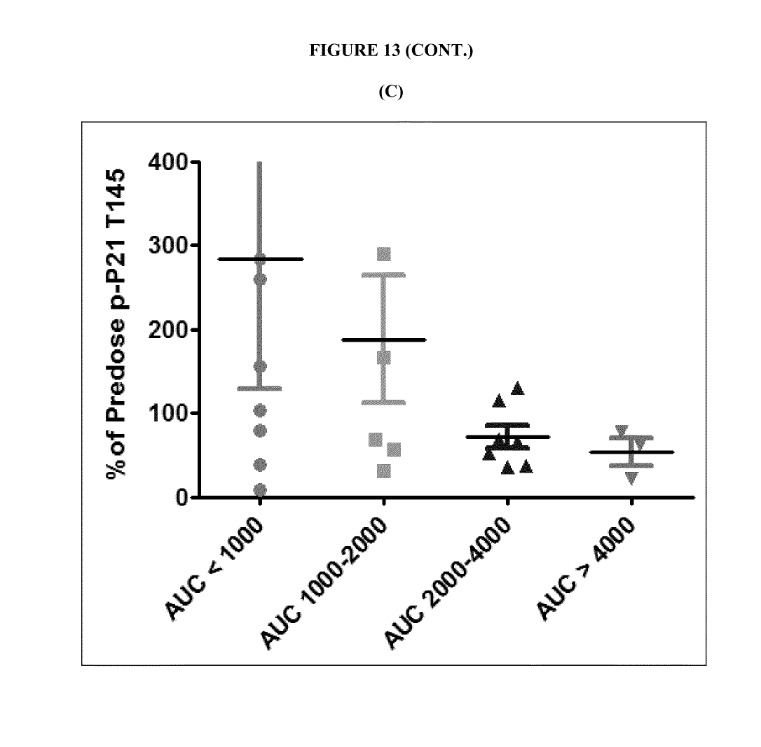

The percentage change in p-Akt S129 was compared from pre-dose (time=0), at 4 hrs, between Day 1 and Day 21 (or Day 1 and Day 8), and as a function of cumulative CX-4945 AUC as shown in FIG. 13A.

The percentage change in p-Akt S473 was compared from pre-dose (time=0), at 4 hrs, between Day 1 and Day 21 (or Day 1 and Day 8), and as a function of cumulative CX-4945 AUC as shown in FIG. 13B.

The percentage change in p-p21 T145 was compared from pre-dose (time=0), at 4 hrs, between Day 1 and Day 21 (or Day 1 and Day 8), and as a function of cumulative CX-4945 AUC as shown in FIG. 13C.

As shown in FIGS. 13A-C, phosphorylation of the biomarkers Akt-S 129, Akt-S473, and p21-T145 decreases in a clear exposure-related (AUC) manner. Moreover, this data demonstrates that CX-4945 is affecting the CK2-specific biomarker Akt-S129 in PBMCs and indicates that CX-4945 is having a signficant impact on its target molecule CK2.

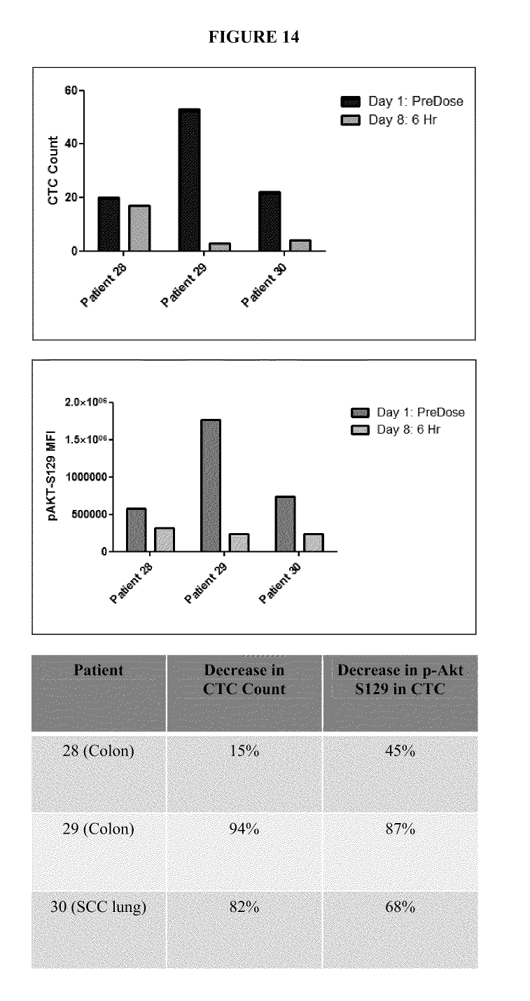

In addition, circulating tumor cells (CTCs) were isolated to analyze p-Akt-S129 at predose (time=0) on Day 1 and 6 hours post dose on Day 8 for patients on the QID schedule. The percentage change in the number of CTC and the p-Akt-S129 measure in the CTC was compared from pre-dose (time=0), on Day 1 and 6 hours on Day 8 as shown in FIG. 14.

### Example 2

### Effect of CK2 Inhibitor on IL-6 Secretion by Inflammatory Breast Cancer Cells

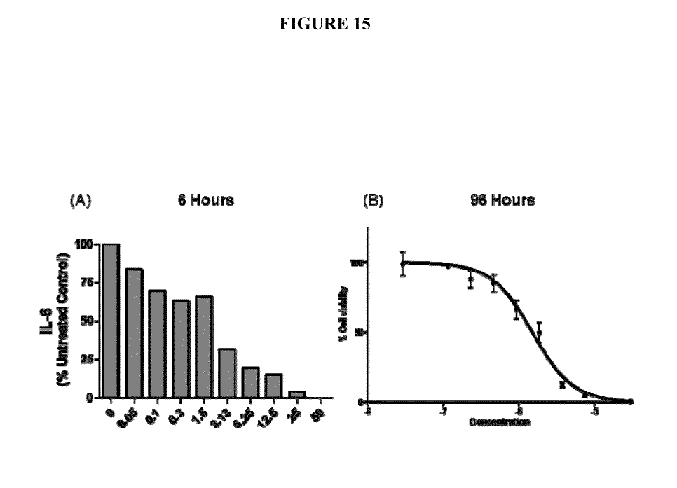

The secretion of IL-6 by SUM-149PT inflammatory breast cancer (IBC) cells was evaluated as a function of CK2 inhibitor concentration. IL-6 levels as a percent of untreated control were determined at 6 hours with CX-4945 at concentrations from 0.05 μM up to 50 μM. Cell viability of the SUM-149PT cells was determined after 96 hours. Results are shown in FIG. 15.

### Example 3

### Effect of CK2 Inhibitor on IL-6 Secretion by Aggressive Inflammatory Breast Cancer Xenografts

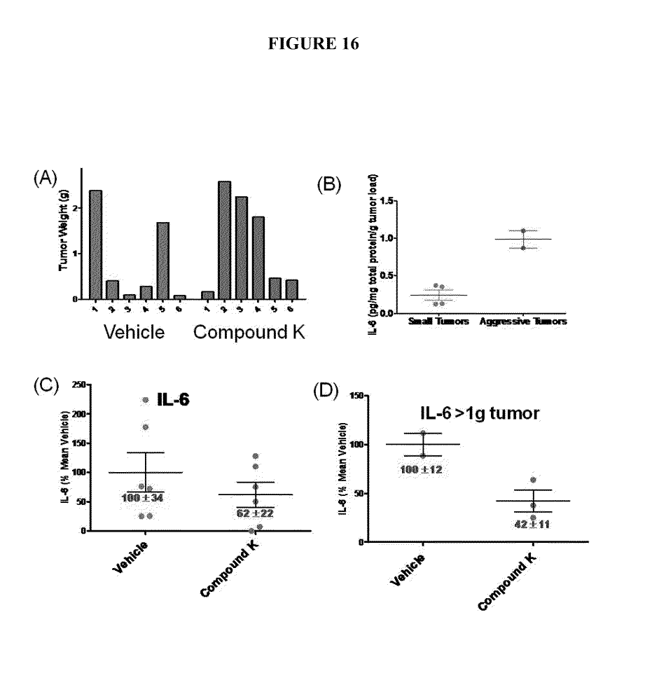

The effect of CK2 inhibitors on the secretion of IL-6 by aggressive SUM-149PT xenografts was also studied. Aggressive tumors (larger than 1 g) were found to have a higher rate of IL-6 secretion than smaller tumors (FIG. 16B).

CX-4945 was found to significantly reduce IL-6 secretion by aggressive tumors (FIG. 16D).

### Example 4

### In Vivo Study in Mice Bearing SUM-149PT Xenografts

Mice bearing SUM-149PT xenografts were left untreated (UTC) or were treated PO once (one time) or BID×8 days (×D8) with 75 mg/kg of CX-4945.

Plasma was isolated, tumors were extracted and weighted. Human IL-6 levels in plasma were determined by ELISA and resulted values were normalized for tumor weight.

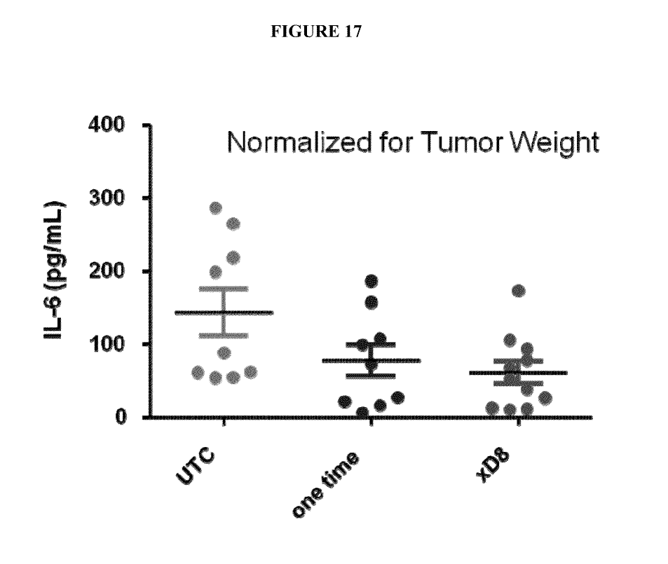

Both single dose and BID×8d treatments resulted in dramatic reduction of human IL-6 levels in animals' plasma (46 and 58% respectively), as shown in FIG. 17.

### Example 5

### The Phosphorylation Status of Akt S129 is a CK2 Specific Biomarker

The S129 site of Aktl was found to be unique to CK2 using the Scansite 2.0 software. See Obenauer et al., Scansite 2.0: Proteome-wide prediction of cell signaling interactions using short sequence motifs, 2003, Nucl Acids Res 31: 3635-41.

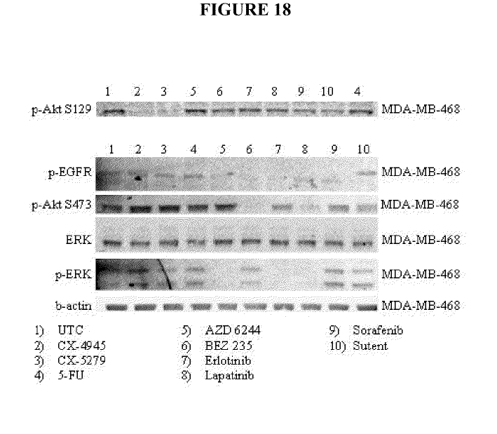

To evaluate the effect of CX-4945 on the phosphorylation status of Akt S129, expression of Akt S129 was measured in untreated cells (UTC) and a compared to cells treated with CX-4945 and a number of other chemotherapeutic agents, including 5-fluorouracil (5-FU), BEZ 235, a PIK3/mTOR dual inhibitor, AZD 6244, a MEK inhibitor, erlotinib, an EGFR tyrosine kinase inhibitor, lapatinib, an EGFR and Her2 dual inhibitor, sorafenib, a multi-targeted RTK (Raf, PDGF, VEGF, C-Kit), and sunitinib (Sutent), a multi-targeted RTK. As shown in FIG. 18, the p-Akt S129 marker responds early to treatment with CX-4945. These results were validated in cell culture, in mouse PBMCs, and in tumor tissue (IHC).

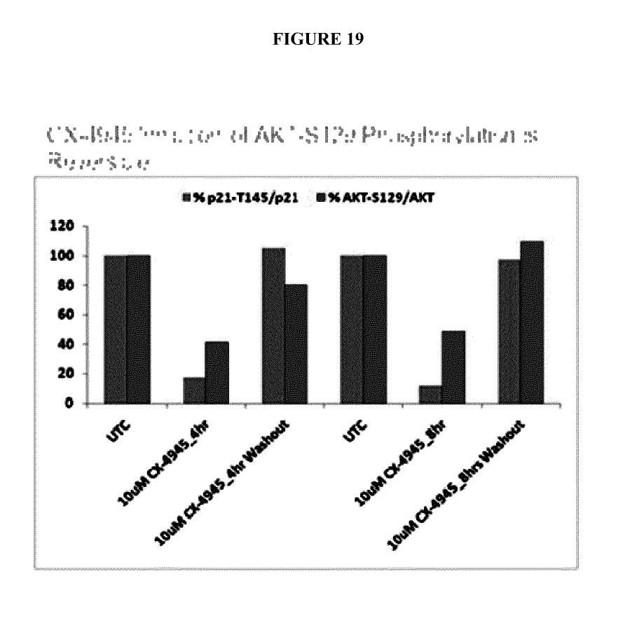

In addition, CX-4945 inhibition of Akt S129 phosphorylation was found to be reversible. See FIG. 19. These data suggest that the phosphorylation status of Akt S129 can be used to monitor the response of a cancer cell to a CK2 inhibitor.

### Example 6

### CK2 Subunit Expression and Sensitivity to CK2 Inhibitors

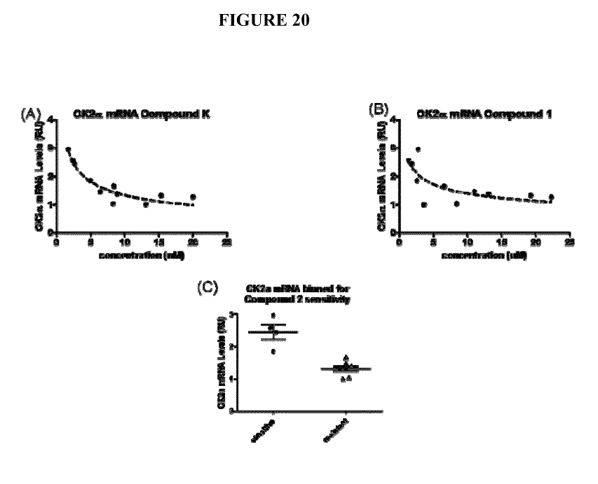

CK2α mRNA levels were determined in breast cancer cells using standard methods. Breast cancer cells with higher CK2α mRNA levels were found to be more sensitive toward CK2 inhibitors, as shown in FIG. 20 for breasts cancer cells treated with CX-4945 (A), Compound 1 (B) and Compound 2 (C).

The correlation between CK2 subunit expression, Akt S129 phosphorylation status and sensitivity to CX-4945 and Compound 2 was analyzed.

A direct correlation was identified between CK2α mRNA expression levels and the activity of several CK2 inhibitors in cancer cells. Breast cancer cells with higher CK2α mRNA levels were found to be more sensitive to Compound K (CX-4945) and other CK2 (e.g. Compound 1 and Compound 2) inhibitors than cells with lower levels of CK2α expression (see FIG. 20).

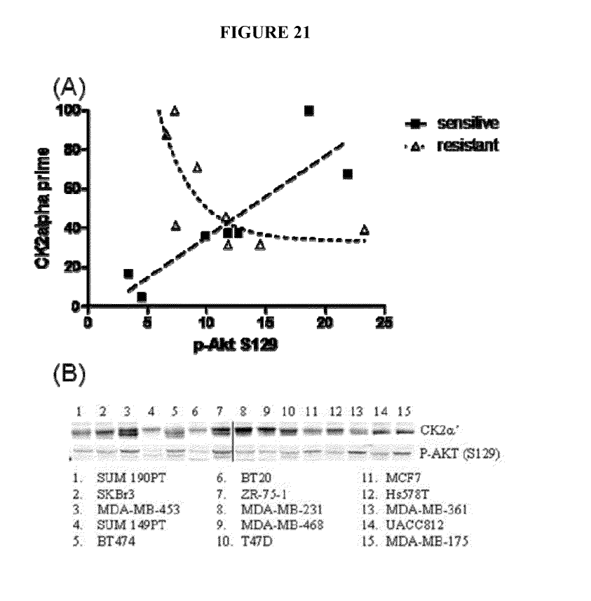

In breast cancer cell lines sensitive to CX-4945 and Compound 2, the phosphorylation status of Akt S129 was directly proportional to CK2α′ expression. In breast cancer cell lines resistant to CX-4945 and Compound 2, the phosphorylation status of Akt S129 was a multiplicative inversely proportional to CK2α′ expression. Results are shown in FIG. 21.

Phosphoprotein levels decreased with increasing exposure to the CK2 inhibitors, as measured by cumulative AUC, demonstrating inhibition of intracellular CK2 activity.

Accordingly, analysis of the relationship between CK2 catalytic subunit expression and Akt S129 phosphorylation status can therefore be used to predict the sensitivity of cancer cells toward CK2 inhibitors, such as CX-4945.

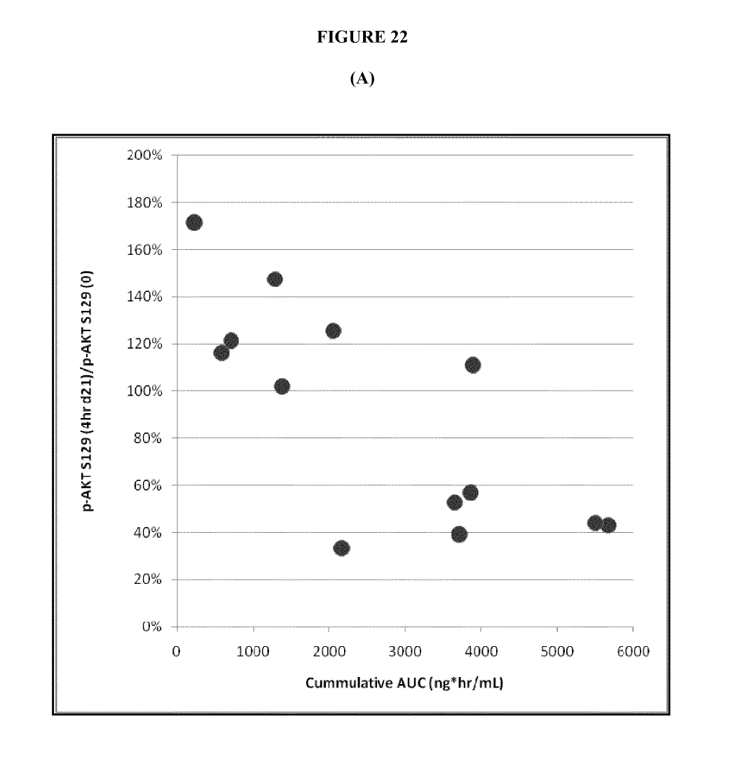

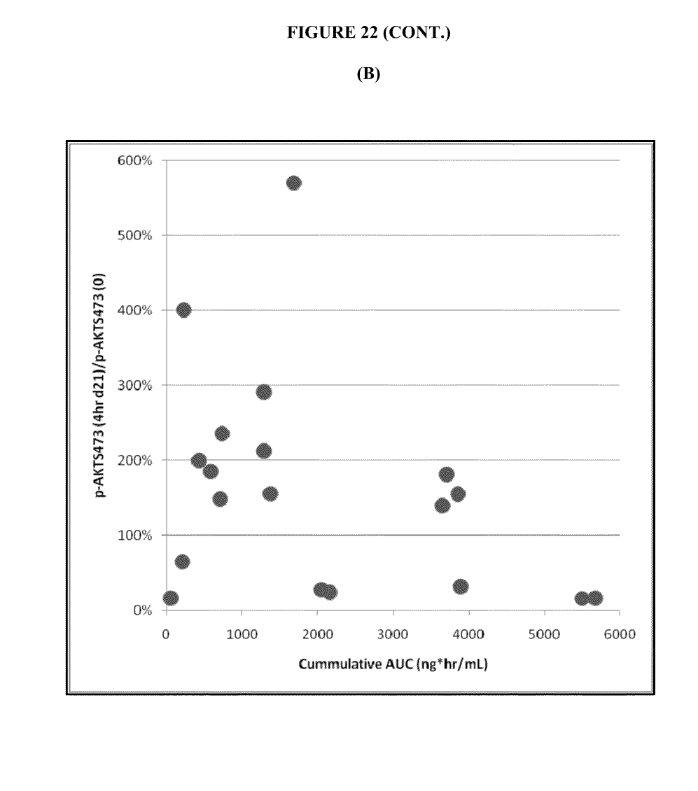

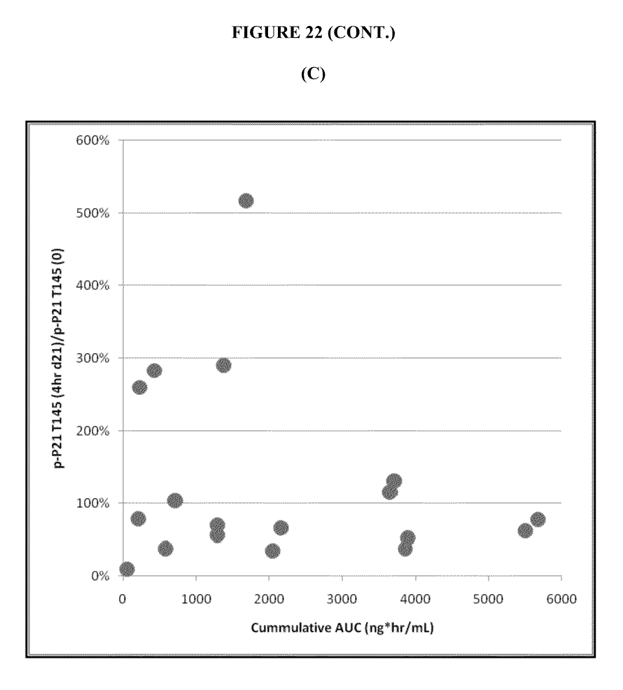

In addition, phosphorylation of the biomarkers Akt S129, Akt S473 and p21 T145 in the PI3 pathway was shown to decrease in an exposure related (AUC) manner (FIGS. 22A-C), indicating that the phosphorylation status of Akt S129, Akt S473 and p21 T145 can be used to monitor the response of the CK2-mediated disease to treatment with a CK2 inhibitor.

### Example 7

### Analysis to Determine Markers Influencing Sensitivity to CK2 Inhibitors

Expression levels of the CK2α subunit, p-Akt S129 and total Akt1 were determined using standard techniques. The usefulness of these markers to predict the IC₅₀ values for CK2 inhibitors in cancer cells was assessed.

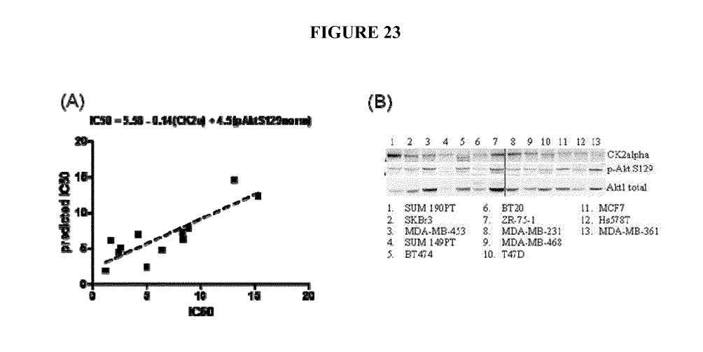

The IC₅₀ of CX-4945 was best predicted by examining the relative expression of CK2α and Akt S129 phosphorylation status normalized to total Akt expression, according to the expression: IC₅₀=5.58−0.14 (CK2α)+4.5(pAktS129ₙₒᵣₘ). See FIG. 23.

### Example 8

### CX-4945 Modulates PI3K/Akt Signaling and Cell Cycle Progression

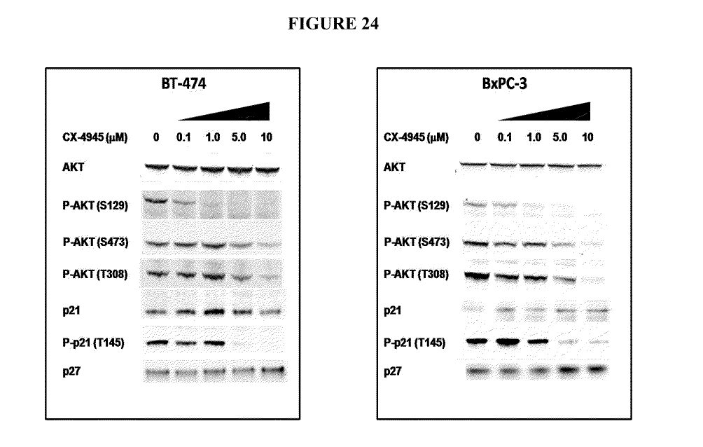

The effect of increasing concentrations of CX-4945 on PIK3/Akt signaling and cell cycle progression was evaluated in BT-474 breast cancer and BxPC-3 pancreatic cancer cells. As shown in FIG. 24, CX-4945 reduced the levels of p-Akt S129 and p-Akt S473, as well as p-p21 T145 in both cell types.

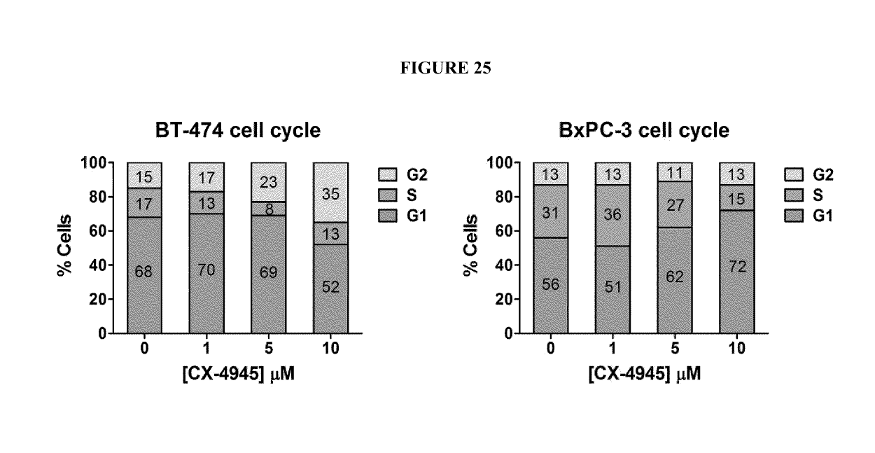

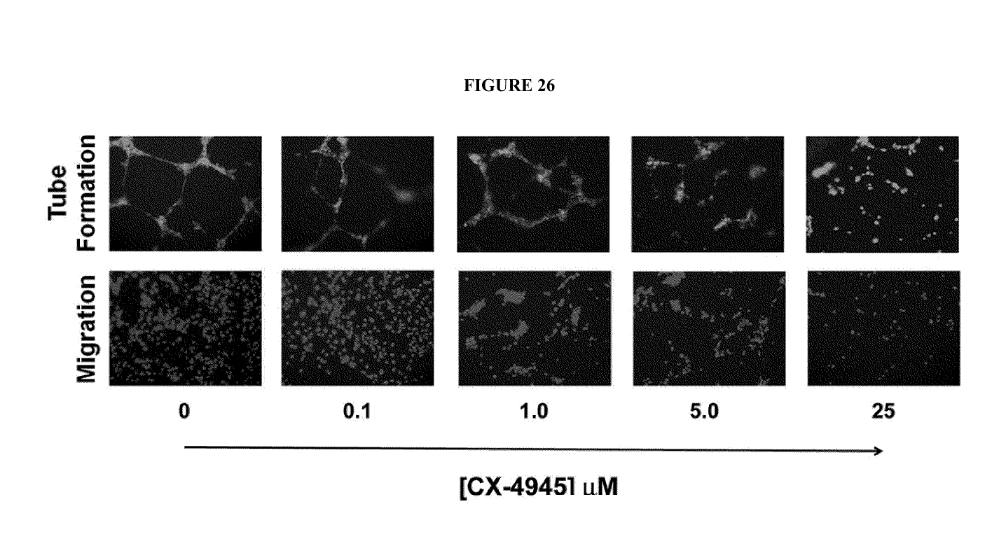

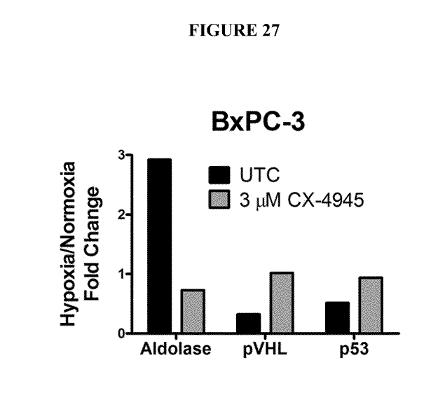

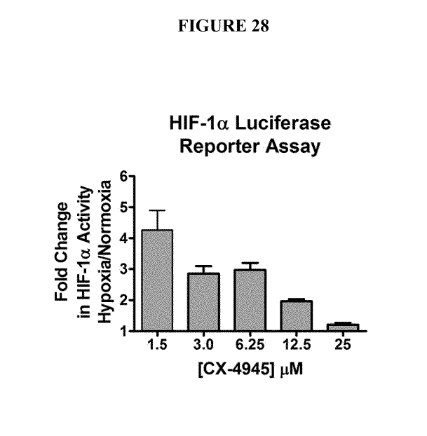

As seen in FIG. 25, CX-4945 modulates the cell cycle in both BT-474 and BxPC-3 cancer cells. With respect to angiogenesis and hypoxia, increasing concentrations of CX-4945 were seen to have significant effects on tube formation and migration in BxPC-3 cells. See FIG. 26. Concentrations of aldolase were reduced following treatment with CX-4945, while levels of pVHL and p53 were increased. See FIG. 27. Using a luciferase reporter assay to measure the expression of hypoxia-inducible factor-1α (HIF-1α), decreasing activity of HIF-1α was seen following exposure to increasing concentrations of CX-4945 (FIG. 28).

### Example 9

### CK2 is Overexpressed in a Panel of Human Multiple Myeloma Cell Lines

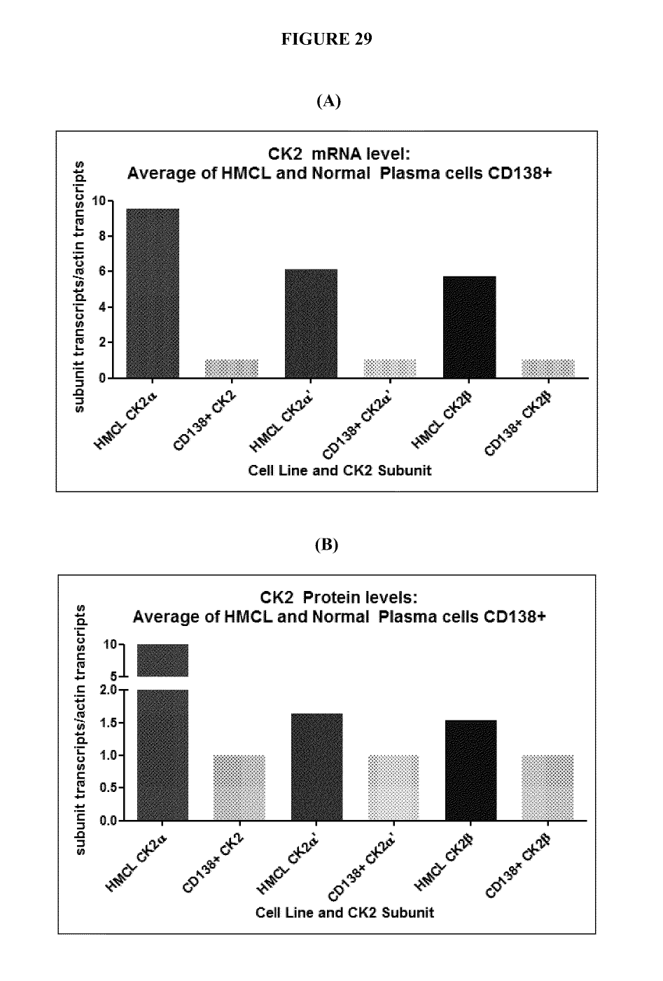

The mRNA and protein levels of CK2 were evaluated in HMCL (Human Myeloma Cell Line) and normal plasma cells CD138+. The levels of CK2α, CK2α′, and CK2β were measured and normalized with actin transcripts. As shown in FIG. 29, the mRNA and protein levels of CK2α, CK2α′, and CK2β were elevated in the multiple myeloma cell lines as compared to normal plasma cells.

### Example 10

### CX-4945 Reduces CK2 Kinase Activity in Multiple Myeloma Cell Lines

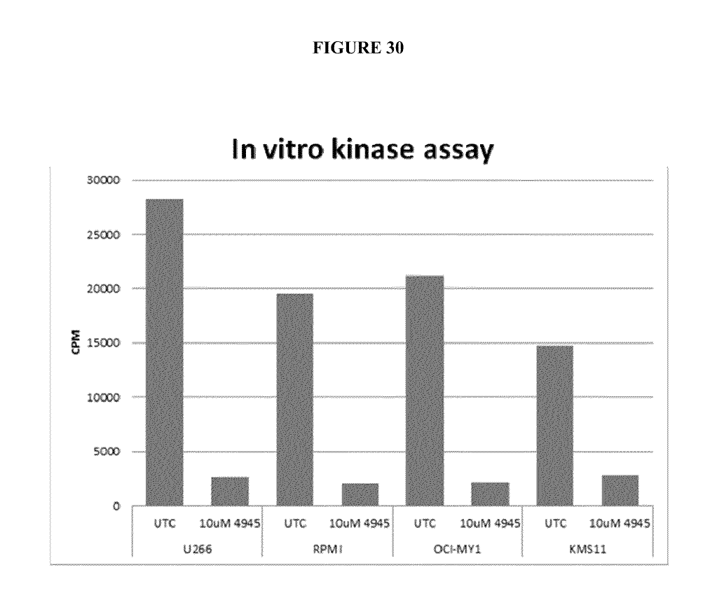

An in vitro kinase assay was performed to measure the CK2 kinase activity in multiple myeloma cell lines following treatment with 10 μM of CX-4945. As shown in FIG. 30, CX-4945 significantly reduced CK2 kinase activity in U266, RPMI, OCI-MY1, and KMS11 multiple myeloma cells lines as compared to untreated cells (UTC).

### Example 11

### CX-4945 Modulates CK2 Signaling in Human Multiple Myeloma Cells

This example demonstrates that CX-4945 exhibits mediates several activities including multiple myeloma cells, including the reduction of Akt-S129, p21-T145, NF-κB, and JAK/STAT phosphorylation, the reduction of IL-6 levels, and induction of cell apoptosis. In addition, CX-4945 inhibits hypoxia induced HIF-1α and suppresses VEGF. Responses assessed in this study include determination of the levels of the following markers: p-p21, p-Akt, IL-6, IL-8, Ki67, Caspase, CTC and FDG-PET.

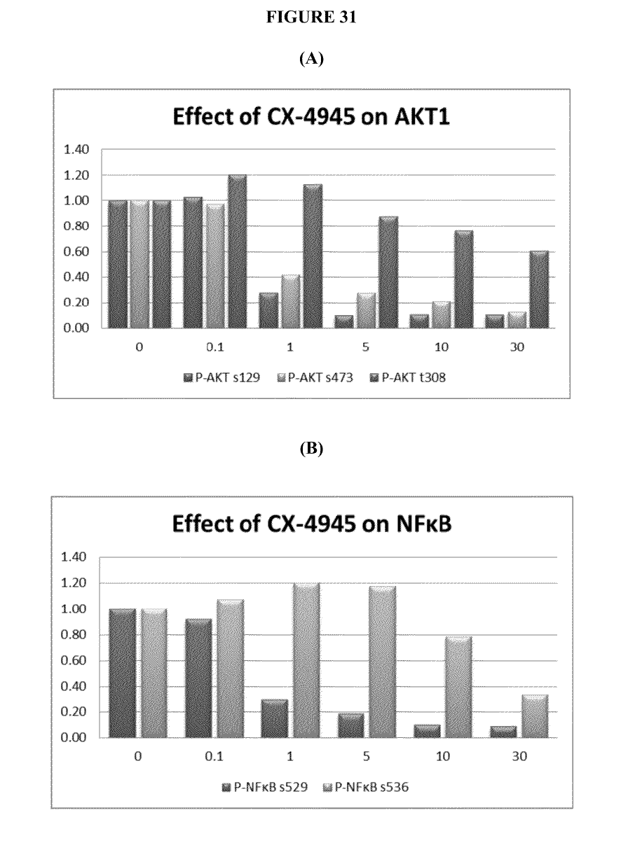

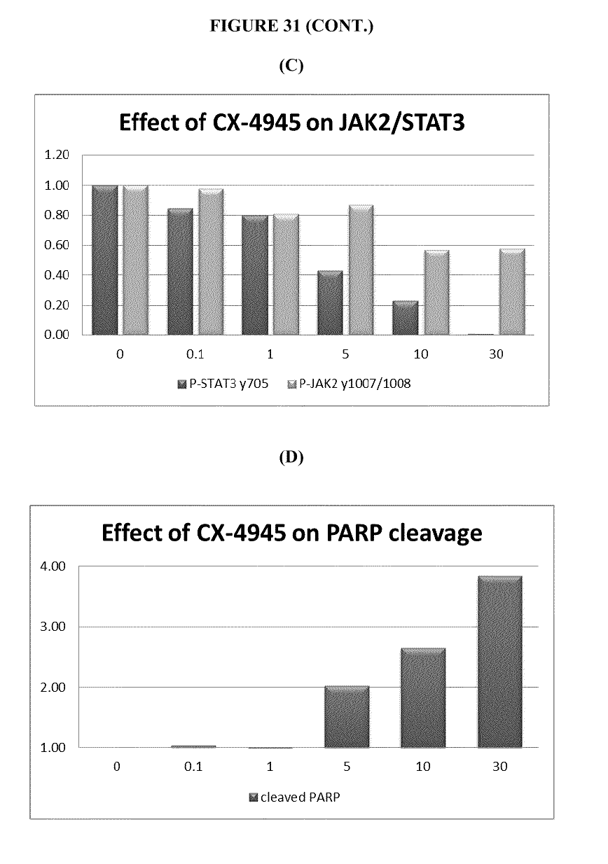

The effect of CX-4945 on CK2 signaling was measured in human multiple myeloma cells. Specifically, CX-4945's effect on Akt1 and NF-κB phosphorylation, JAK/STAT modulation, and PARP cleavage was evaluated. See FIGS. 31A-D. CX-4945 reduced the phosphorylation of p-Akt S129 and S473 (FIG. 31A), as well as the phosphorylation of p-NF-κB S529 (FIG. 31B). Moreover, CX-4945 was shown to reduce the phosphorylation of p-STAT3 Y705 and p-JAK2 Y1007/1008 (FIG. 31C), and was seen to increase PARP cleavage (FIG. 31D), a marker for cell apoptosis.

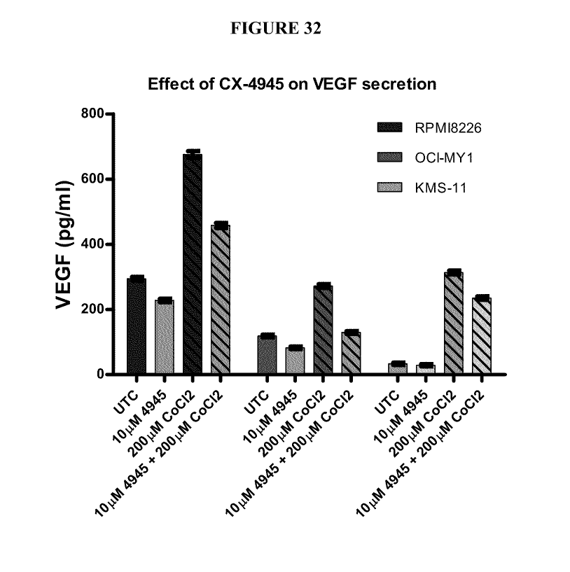

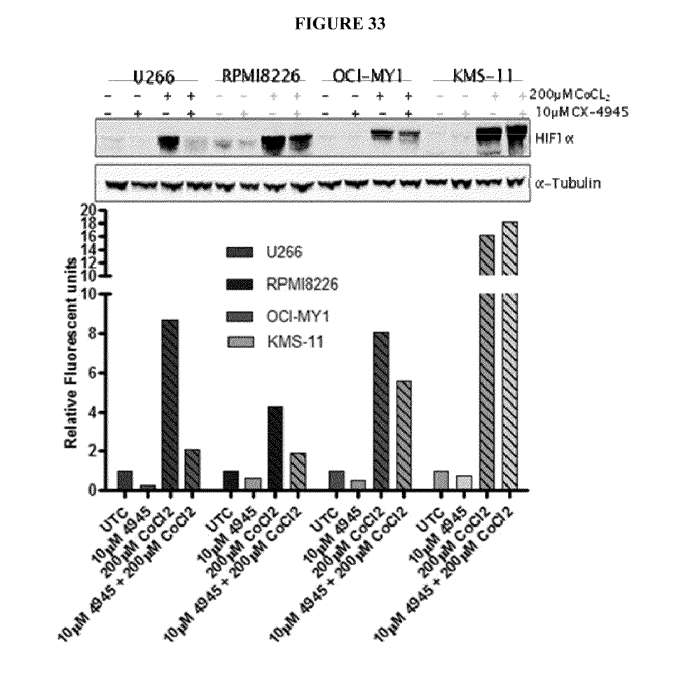

In addition, the effect of CX-4945 on VEGF secretion was examined. As shown in FIG. 32, treatment with 10 μM CX-4945 reduced the secretion of VEGF in multiple myeloma cell lines. Moreover, CX-4945 was seen to modulate the expression of HIF-1α in a panel of multiple myeloma cell lines. See FIG. 33.

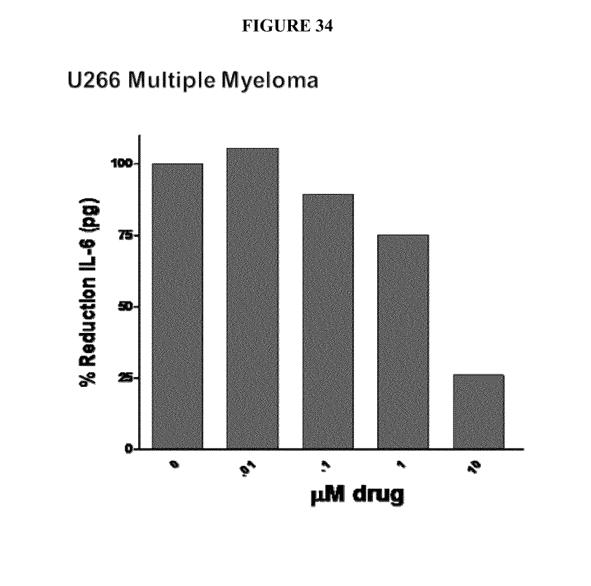

Lastly, treatment with CX-4945 in U266 multiple myeloma cells was shown to reduce the production of IL-6, a key growth and survival factor for myeloma cells as well as a major morbidity factor for patients with multiple myeloma. See FIG. 34.

Because CX-4945 reduces CK2 activity in multiple myeloma cells, it has the effect of modulating the activity of several key proteins in this disease. Specifically, CK2 phosphorylates multiple substrates in the PI3K/Akt pathway including Akt-S129 which is exclusively phosphorylated by CK2. In addition, CK2 modulates JAK/STAT, and phosphorylates NF-κB including NF-κB S529. Moreover, CK2 suppresses cell apoptosis and is elevated under hypoxia.

### Example 12

### Further Investigation of CX-4945 Activity in the PI3K/Akt Pathway

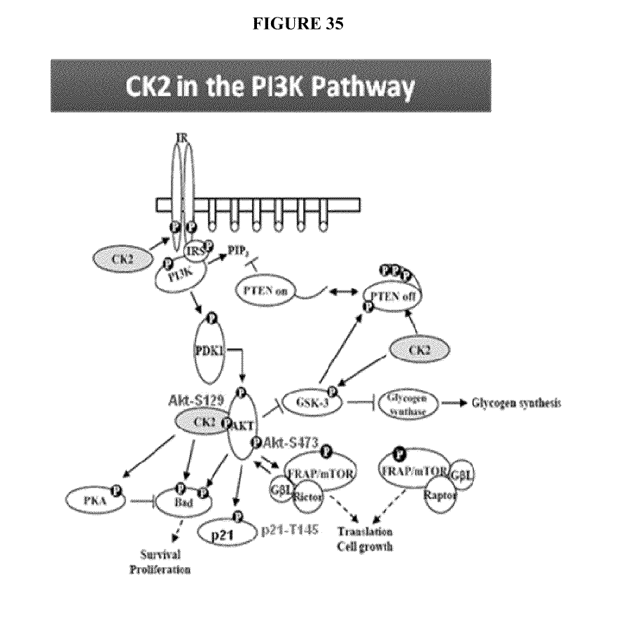

As described above, CK2 phosphorylates multiple substrates in the PI3K/Akt pathway. See FIG. 35. The present inventors have shown that Akt-S129 is exclusively phosphorylated by CK2.

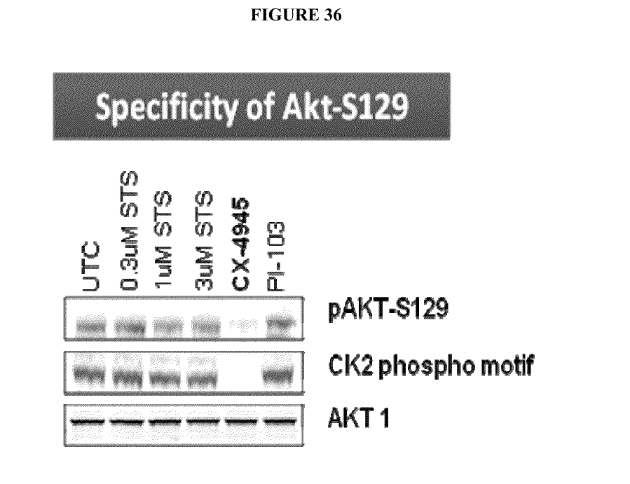

In this example, the ability of the CK2 inhibitor, CX-4945, to inhibit phosphorylation of Akt-129 was compared to that of staurosporine (STS), another kinase inhibitor. Interestingly, CX-4945 inhibited phosphorylation of Akt-S129, while exposure to STS did not affect the phosphorylation of Akt-5129. See FIG. 36.

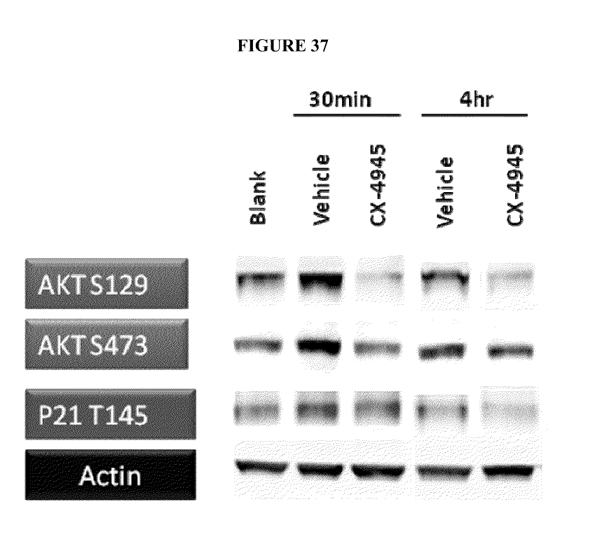

To further investigate the ability of CX-4945 to reduce the phosphorylation of various targets of the PIK3/Akt pathway, the compound was administered orally to mice (75 mg/kg bid) and the phosphorylation of Akt-5129, Akt-5473, and p21-T145 was evaluated in mouse PBMCs. As shown in FIG. 37, the phosphorylation of Akt-S129, Akt-S473, and p21-T145 was reduced in mice treated with CX-4945.

### Example 13

### CX-4945 Combinations with DNA-Damaging Chemotherapeutic Agents

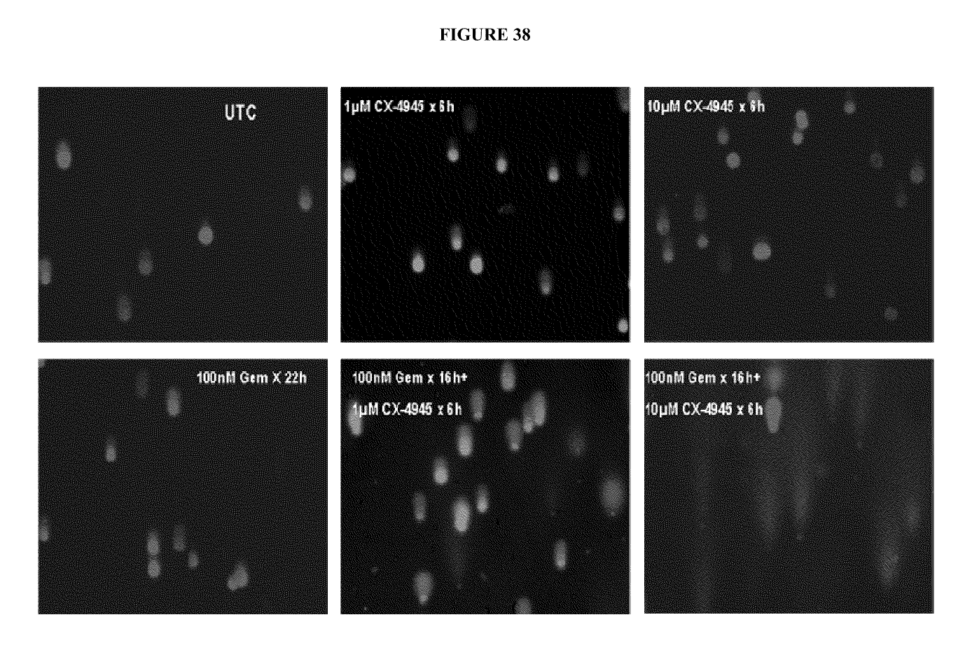

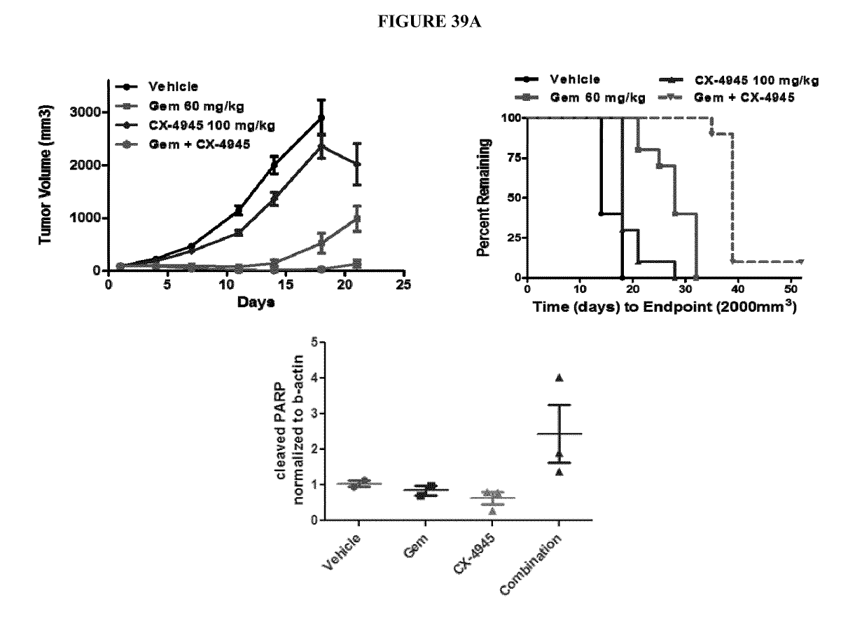

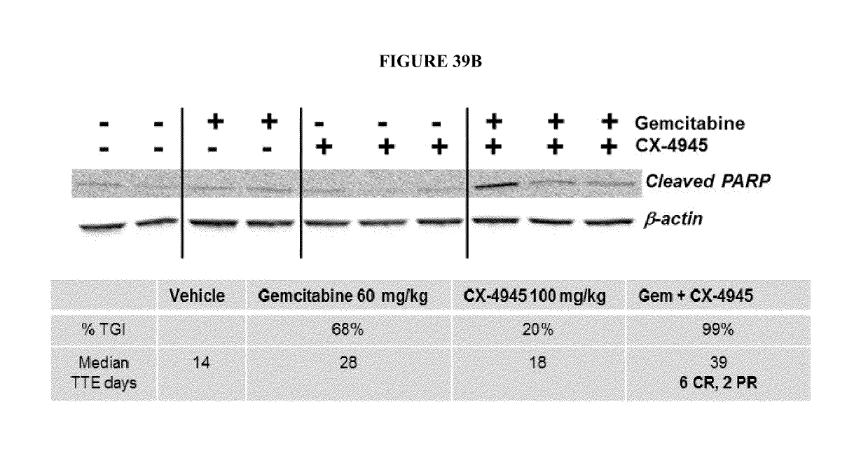

Using a comet assay, CX-4945 was seen to increase gemcitabine induced DNA damage in A2780 ovarian cancer cells. See FIG. 38. In addition, gemcitabine and CX-4945 exhibited synergistic anti-tumor activity in A2780 xenografts. See FIGS. 39A and 39B.

### Example 14

### CX-4945 Combinations with EGFR Targeting Agents

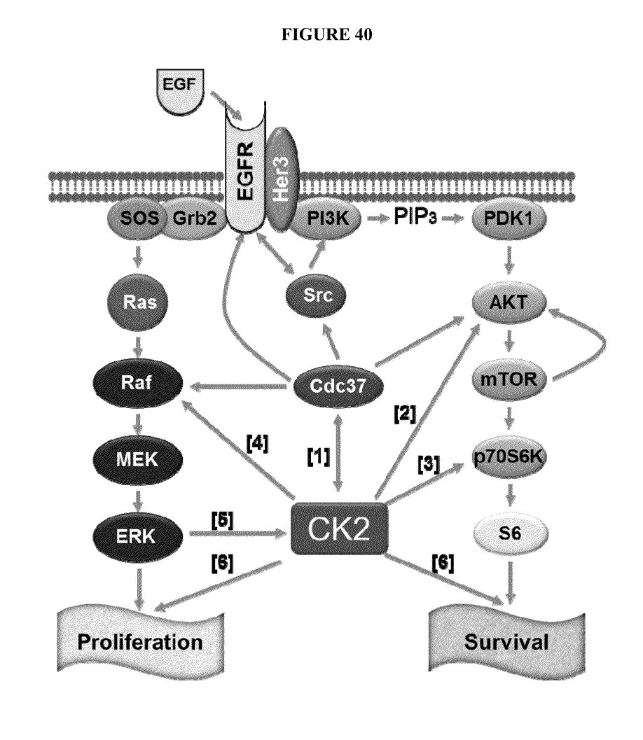

As shown in FIG. 40, crosstalk exists between EGFR and CK2 signaling. Specifically, CK2 controls multiple protein kinases by phosphorylating a kinase-targeting molecular chaperone, Cdc37, which exerts effects on EGFR directly, as well Src, which subsequently interacts with EGFR. In addition, nuclear export of S6K1 II is regulated by CK2 phosphorylation of at Ser-17, while EGF-induced ERK activation promotes CK2-mediated dissociation of alpha-catenin from beta-catenin and transactivation of beta-catenin. Lastly, C2K is a component of the KSR1 scaffold complex that contributes to Raf kinase activation.

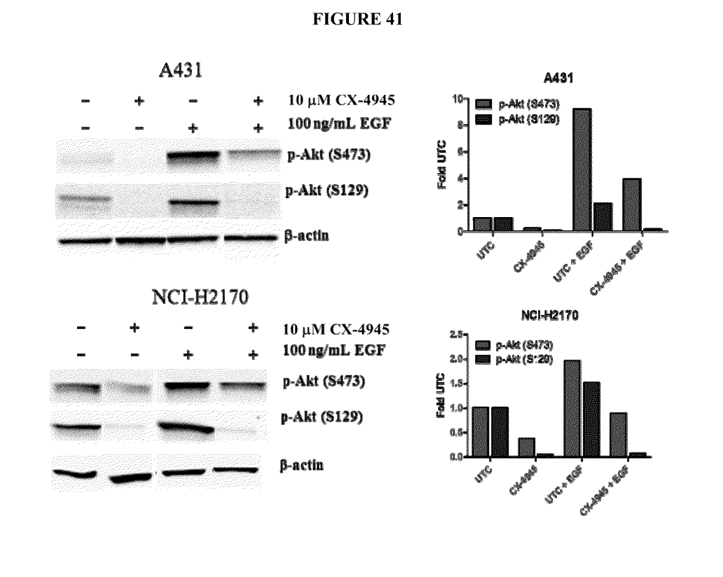

To examine the effect of CX-4945 on epidermal growth factor (EGF)-stimulated CK2 activity, A431 (epidermoid carcinoma) and NCI-H2170 (lung cancer cells) were treated with 100 ng/ml EGF and/or 10 μM CX-4945 and p-Akt S129 and p-Akt S473 levels were measured. In both cell types, CX-4945 was seen to significantly inhibit EGF-stimulated CK2 activity. See FIG. 41.

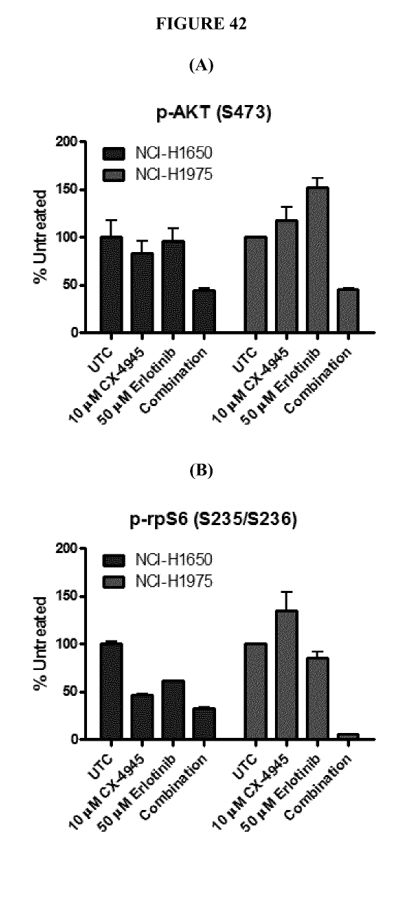

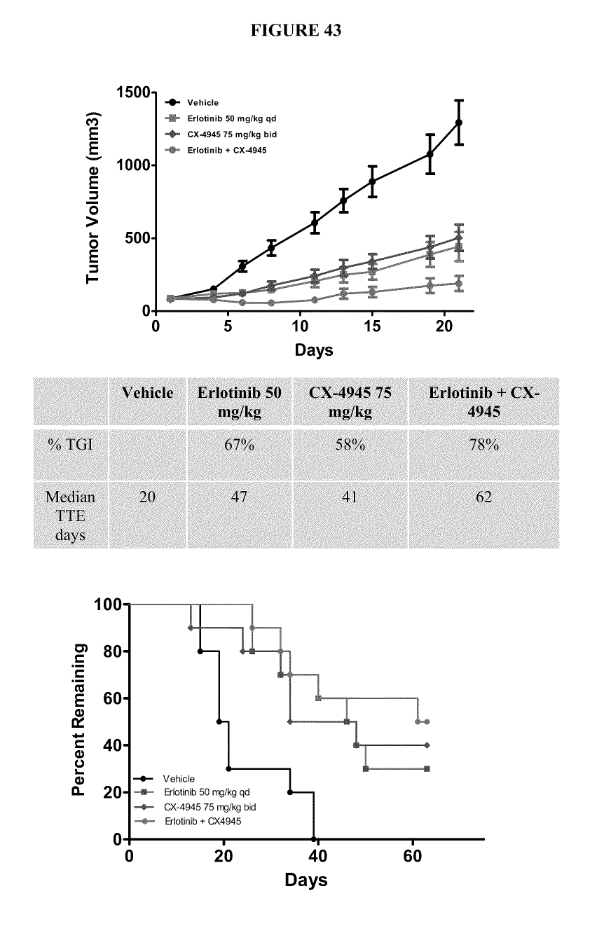

As shown in FIGS. 42A and 42B, the combination of CX-4945 with erlotinib reduces the phosphorylation of Akt and rpS6. Erlotinib targets the epidermal growth factor receptor (EGFR) and is used to treat NSCLC and pancreatic cancer, amongst other cancer types. In A431 xenografts, erlotinib and CX-4945 exhibited synergistic anti-tumor activity. See FIG. 43. Although both erlotinib and CX-4945 showed significant inhibition of tumor growth when used alone, the combination was even more potent, showing synergistic activity which prolonged the time to endpoint.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

The preceding examples are provided to illustrate the invention and do not limit or define its scope. Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

1. A method for monitoring the response of a subject being treated with a CK2 inhibitor, said method comprising:
(a) determining the level of a biomarker in a biological sample derived from the subject at a time point .during or after administration of the CK2 inhibitor, wherein the biomarker is selected from the level of phosphorylated Akt S129, the ratio of phosphorylated Akt S 129 to total Akt, the level of phosphorylated Akt S473, the ratio of phosphorylated Akt S 473 to total Akt, the level of phosphorylated p21 T145, the ratio of phosphorylated p21 T 145 to total p21, the level of phosphorylated NF-κB S529, the ratio of phosphorylated NF-κB S529 to total NF-κB, the level of phosphorylated STAT3 T705, the ratio of phosphorylated STAT3 T705 to total STAT3, the level of phosphorylated JAK2 Y1007/1008, and the ratio of phosphorylated JAK2 Y1007/1008 to total JAK2; and
(b) comparing the level of the biomarker in the biological sample with a reference level of the biomarker;
wherein a decrease in the level of the biomarker in the biological sample compared to the reference level of the biomarker is indicative of a positive response to treatment with said CK2 inhibitor.

2. The method of claim 1, wherein the reference level of the biomarker is selected from the group consisting of (1) the level of said biomarker from the subject prior to administration of the CK2 inhibitor; (2) the level of said biomarker from a reference population; (3) a pre-assigned level for said biomarker; and (4) the level of said biomarker from the subject at a second time point prior to the first time point.

3. The method of claim 1, wherein said biological sample is selected from a cell, a tissue, a tissue culture, a tumor, or a biological fluid derived from said subject.

4. The method of claim 3, wherein said biological fluid is selected from plasma, serum, or PBMCs.

5. The method of claim 3, wherein said cell is a circulating tumor cell (CTC).

6. The method of claim 1, wherein said subject suffers from a cancer or malignancy.

7. The method of claim 6, wherein said cancer or malignancy is selected from breast cancer, inflammatory breast cancer (IBC), pancreatic cancer, prostate cancer, lung cancer, colon cancer, melanoma, and multiple myeloma.

8. The method of claim 1, wherein said subject suffers from a CK-2 mediated autoimmune, inflammatory, or infectious disorder.

9. The method of claim 1, wherein said CK2 inhibitor is CX-4945.

10. A method for monitoring the response of a subject being treated with a CK2 inhibitor, said method comprising:
(a) determining the level of mRNA and/or protein expression of a biomarker in a biological sample derived from the subject at a time point during or after administration of the CK2 inhibitor, wherein the biomarker is selected from IL-6, IL-8, CK2α, CK2α′, CK2β, VEGF, and HIF-1α; and
(b) comparing the level of the biomarker in the biological sample with a reference level of the biomarker;
wherein a decrease in the level of the biomarker in the biological sample compared to the reference level of the biomarker is indicative of a positive response to treatment with said CK2 inhibitor.

11. The method of claim 10, wherein the reference level of the biomarker is selected from the group consisting of (1) the level of said biomarker from the subject prior to administration of the CK2 inhibitor; (2) the level of said biomarker from a reference population; (3) a pre-assigned level for said biomarker; and (4) the level of said biomarker from the subject at a second time point prior to the first time point.

12. The method of claim 10, wherein said biological sample is selected from a cell, a tissue, a tissue culture, a tumor, or a biological fluid derived from said subject.

13. The method of claim 12, wherein said biological fluid is selected from plasma, serum, or PBMCs.

14. The method of claim 12, wherein said cell is a circulating tumor cell (CTC).

15. The method of claim 10, wherein said subject suffers from a cancer or malignancy.

16. The method of claim 15, wherein said cancer or malignancy is selected from breast cancer, inflammatory breast cancer (IBC), pancreatic cancer, prostate cancer, lung cancer, colon cancer, melanoma, and multiple myeloma.

17. The method of claim 10, wherein said subject suffers from a CK-2 mediated autoimmune, inflammatory, or infectious disorder.

18. The method of claim 10, wherein said CK2 inhibitor is CX-4945.

19. A method for predicting the clinical response of a CK2-mediated disease to treatment with a CK2 inhibitor in a subject, said method comprising determining the level of one or more biomarkers in a biological sample derived from the subject, wherein an elevated level of said one or more biomarkers relative to a control biological sample is indicative of sensitivity of the CK2-mediated disease to treatment with said CK2 inhibitor, and wherein said biomarker is selected from the level of phosphorylated Akt S129, the ratio of phosphorylated Akt S 129 to total Akt, the level of phosphorylated Akt S473, the ratio of phosphorylated Akt S 473 to total Akt, the level of phosphorylated p21 T145, the ratio of phosphorylated p21 T145 to total p21, the level of phosphorylated NF-κB S529, the ratio of phosphorylated NF-κB S529 to total NF-κB, the level of phosphorylated STAT3 T705, the ratio of phosphorylated STAT3 T705 to total STAT3, the level of phosphorylated JAK2 Y1007/1008, the ratio of phosphorylated JAK2 Y1007/1008 to total JAK2, the expression level of IL-6, the expression level of IL-8, the expression level of CK2α, the expression level of CK2α′, the expression level of CK2β, the expression level of VEGF, and the expression level of HIF-1α.

20. The method of claim 19, wherein said biological sample is selected from a cell, a tissue, a tissue culture, a tumor, or a biological fluid derived from said subject.

21. The method of claim 20, wherein said biological fluid is selected from plasma, serum, or PBMCs.

22. The method of claim 20, wherein said cell is a circulating tumor cell (CTC).

23. The method of claim 19, wherein said subject suffers from a cancer or malignancy.

24. The method of claim 23, wherein said cancer or malignancy is selected from breast cancer, inflammatory breast cancer (IBC), pancreatic cancer, prostate cancer, lung cancer, colon cancer, melanoma, and multiple myeloma.

25. The method of claim 19, wherein said subject suffers from a CK-2 mediated autoimmune, inflammatory, or infectious disorder.

26. The method of claim 19, wherein said CK2 inhibitor is CX-4945.

27. A method for predicting the clinical response of a cancer or malignancy to treatment with a CK2 inhibitor in a subject, said method comprising:
(a) determining the level of CK2α′ mRNA and/or protein expression in a biological sample derived from the subject; and
(b) determining the level of p-Akt S129 and/or the ratio of p-Akt S129 to total Akt in a biological sample derived from the subject;
wherein a positive correlation between the level of CK2α′ mRNA and/or protein expression and the level of p-Akt S129 and/or ratio of p-Akt S129 to total Akt is indicative of sensitivity of the cancer or malignancy to treatment with said. CK2 inhibitor.

28. The method of claim 27, wherein said biological sample is selected from a cell, a tissue, a tissue culture, a tumor, or a biological fluid derived from said subject.

29. The method of claim 28, wherein said biological fluid is selected from plasma, serum, or PBMCs.

30. The method of claim 27, said cancer or malignancy is breast cancer, inflammatory breast cancer (IBC), or multiple myeloma.

31. The method of claim 27, wherein said CK2 inhibitor is CX-4945.
